(19) United States
(12) Patent Application Publication
(10) Pub. No.: US 2021/0284695 A1
(43) Pub. Date: Sep. 16, 2021

(54) FOLDED AND PROTEASE-RESISTANT POLYPEPTIDES

(71) Applicant: UNIVERSITY OF WASHINGTON, Seattle, WA (US)

(72) Inventors: Gabriel Jacob ROCKLIN, Seattle, WA (US); David BAKER, Seattle, WA (US)

(21) Appl. No.: 16/489,044

(22) PCT Filed: Apr. 27, 2018
(86) PCT No.: PCT/US2018/029904

§ 371 Date: Aug. 27, 2019

Related U.S. Application Data
(60) Provisional application No. 62/491,518, filed on Apr. 28, 2017.

(51) Int. Cl. C07K 14/00; C12N 15/10

(52) U.S. Cl. CPC C07K 14/00; C12N 15/1037; C12N 15/1068

(57) ABSTRACT

Non-naturally occurring polypeptides are disclosed that include (a) 3-3 secondary structure elements, wherein each secondary structure element is either an α-helix (H domain) of between 10-20 amino acid residues in length or a β-strand (E domain) of between 3-10 amino acid residues in length; and (b) 2-4 linkers of between 2 to 6 amino acid residues in length connecting adjacent secondary structure elements; wherein the polypeptide is between 25-50 amino acid residues in length; and wherein the polypeptide includes no cysteine residues.

### CROSS REFERENCE

This application claims priority to U.S. Provisional Patent Application Ser. No. 62/491,518 filed Apr. 28, 2017, incorporated by reference herein in its entirety.

### BACKGROUND

Proteins fold into unique native structures stabilized by thousands of weak interactions that collectively overcome the entropic cost of folding. Though these forces are “encoded” in the thousands of known protein structures, “decoding” them is challenging due to the complexity of natural proteins that have evolved for function, not stability.

The key challenge to achieving a quantitative understanding of the sequence determinants of protein folding is to accurately and efficiently model the balance between the many forces contributing to the free energy of folding. Mutagenesis has been the primary experimental tool for interrogating the balance of forces contributing to stability, but the results are context-dependent and do not provide a global view of the contributions to stability. Molecular dynamics simulations on minimal proteins do not reveal which interactions specify and stabilize the native structure, and cannot in general determine whether a given sequence will fold into a stable structure.

### SUMMARY OF THE INVENTION

In one aspect, non-naturally occurring polypeptides are provided, comprising

(a) 3-5 secondary structure elements, wherein each secondary structure element is either an α-helix (H domain) of between 10-20 amino acid residues in length or a β-strand (E domain) of between 3-10 amino acid residues in length; and

(b) 2-4 linkers of between 2 to 6 amino acid residues in length connecting adjacent secondary structure elements;

wherein the polypeptide is between 25-50 amino acid residues in length; and

wherein the polypeptide includes no cysteine residues.

In one embodiment, each H domain is independently between 10-15 amino acids in length. In another embodiment, each E domain is independently between 3-7 amino acids in length. In a further embodiment, the polypeptide is between 30-50, 35-50, 35-45, 40-45, or 40-43 amino acid residues in length. In another embodiment, the polypeptide comprises a secondary structure element arrangement selected from the group consisting of HHH, EHEE, HEEH, and EEHEE. In various embodiments, the polypeptide comprises an amino acid sequence having at least at least 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to the amino acid sequence of any one of SEQ ID NOS: 1-4000, or a mirror image thereof. Also provided are isolated nucleic acids encoding the polypeptide of any embodiment herein, recombinant expression vectors comprising the isolated nucleic acids linked to a promoter, and recombinant host cells comprising the recombinant expression vectors disclosed herein.

Also provided herein are methods comprising:

(a) using a computing device to construct a library of proteins, wherein the computing device designs a sequence to stabilize the backbone of the protein, and wherein the proteins comprise less than about 50 amino acids;

(b) synthesizing the proteins using next-generation gene synthesis;

(c) expressing the proteins in yeast so that every cell displays many copies of one protein sequence on its surface; and

(d) screening the library of proteins for susceptibility to digestion by protease.

In one embodiment, the synthesizing step comprises oligo library synthesis technology, capable of parallel synthesis of 10⁴-10⁵ arbitrarily specified DNA sequences long enough to encode the proteins. In another embodiment, in the screening step, cells are incubated with varying concentrations of protease, those displaying resistant proteins are isolated by fluorescence-activated cell sorting (FACS), and the frequencies of each protein at each protease concentration are determined by deep sequencing. In a further embodiment, the method further comprising assigning each protein a stability score, wherein the stability score comprises: the difference between the measured EC₅₀ and the predicted EC₅₀ in the unfolded state of the protein, according to a sequence-based model parameterized using EC₅₀ measurements of scrambled sequences. In one embodiment, a stability score of 1 corresponds to a 10-fold higher EC₅₀ than the predicted EC₅₀ in the unfolded state. In another embodiment, the library comprises 1,000 to 30,000 proteins.

### DESCRIPTION OF THE FIGURES

FIG. 1. Yeast display enables massively parallel measurement of protein stability. (A) Each yeast cell displays many copies of one test protein fused to Aga2. The c-terminal c-Myc tag is labeled with a fluorescent antibody. Protease cleavage of the test protein (or other cleavage) leads to loss of the tag and loss of fluorescence. (B) Libraries of 10⁴ unique sequences are sorted by flow cytometry. Most cells show high protein expression (measured by fluorescence) before proteolysis (blue). Only some cells retain fluorescence after proteolysis; those above a threshold (shaded green region) are collected for deep sequencing analysis. (C) Sequential sorting at increasing protease concentrations separates proteins by stability. Each sequence in a library of 19,726 proteins is shown as a grey line tracking the change in population fraction (enrichment) of that sequence, normalized to each sequence's population in the starting (pre-selection) library. Enrichment traces for seven proteins at different stability levels are highlighted in color. (D) EC₅₀s for the seven highlighted proteins in (C) are plotted on top of the overall density of the 46,187 highest-confidence EC₅₀ measurements from design rounds 1-4. (E) Same data as at left, showing that stability scores (EC₅₀ values corrected for intrinsic proteolysis rates) correlate better than raw EC₅₀s between the proteases. (F-I) Stability scores measured in high-throughput correlate with individual folding stability measurements for mutants of four small proteins. The wild-type sequence in each set is highlighted as a red circle. Confidence intervals for all EC₅₀ measurements are provided in supplementary materials. (F) Pin1 ΔG_{unf} data at 40° C. from (28) by thermal denaturation (G) hYAP65 Tm data from (5, 10)(H) Villin HP35 ΔG_{unf} data at 25° C. from (7, 11) by urea denaturation (1) BBL ΔG_{unf} data at 10° C. from (8) by thermal denaturation.

FIG. 2. Biophysical characterization of designed minimal proteins. (A) Design models and NMR solution ensembles for designed minimal proteins. (B) Far-ultraviolet circular dichroism (CD) spectra at 25° C. (black), 95° C. (red), and 25° C. following melting (blue). (C) Thermal melting curves measured by CD at 220 nm. Melting temperatures determined using the derivative of the curve. (D) Chemical denaturation in GuHCl measured by CD at 220 nm and 25° C. Unfolding free energies determined by fitting to a two-state model (red solid line).

### DETAILED DESCRIPTION OF THE INVENTION

All references cited are herein incorporated by reference in their entirety.

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In one aspect, the invention provides non-naturally occurring polypeptides comprising or consisting of:

(a) 3-5 secondary structure elements, wherein each secondary structure element is either an α-helix (H domain) of between 10-20 amino acid residues in length or a β-strand (E domain) of between 3-10 amino acid residues in length; and

(b) 2-4 linkers of between 2 to 6 amino acid residues in length connecting adjacent secondary structure elements;

wherein the polypeptide is between 25-50 amino acid residues in length; and

wherein the polypeptide includes no cysteine residues.

As demonstrated in the examples, the inventors have developed computational methods for de novo design of non-naturally occurring folded protease-resistant peptides that do not include cysteine residues and thus do not rely on disulfide bonds for stability, and the use of these methods to design a large number of exemplary 25-50 residue constrained peptides. The stable polypeptides disclosed herein provide robust starting scaffolds for generating peptides that bind targets of interest using computational interface design or experimental selection methods. Solvent-exposed hydrophobic residues can be introduced without impairing folding or solubility, suggesting high mutational tolerance. Hence it should be possible to reengineer the peptide surfaces, incorporating target-binding residues to construct binders, agonists, or inhibitors.

As used herein, a β-sheet secondary structure element comprises β strands connected laterally by backbone hydrogen bonds. As used herein, an α-helix secondary structure element is a right-handed or left-handed (when D amino acids are involved) helix in which backbone amine groups donate a hydrogen bond to backbone carbonyl groups of amino acids 3-4 residues before it along the primary amino acid sequence of the polypeptide. In various embodiments, the polypeptide comprises or consists of 3-5, 3-4, 4-5, 3, 4, or 5 secondary structure elements. In various non-limiting embodiments, the secondary structure element arrangement of the polypeptide may be selected from the group consisting of HHH, EHE, EEH, HEE, HEEE, EEHE, EHEE, EEEH, HEEH, and EEHEE, wherein H is a helix and E is a beta strand. In specific embodiments, structure element arrangement of the polypeptide may be selected from the group consisting of HHH, EHEE, HEEH, and EEHEE.

In various embodiments, each E domain is independently between 3-10, 3-9, 3-8, 3-7, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, 3, 4, 5, 6, 7, 8, 9, or 10 amino acid residues in length. In a specific embodiment, each E domain is independently 3-7 amino acids in length. In one embodiment, each E domain in the polypeptide is the same length; in another embodiment, not all E domains in the polypeptide are the same length.

In other embodiments, each H domain is independently between 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, 19-20, 10, 11, 12, 13, 14, 15, 16, 17, 18, 19, or 20 amino acid residues in length. In a specific embodiment, each H domain is independently between 10-15 amino acid residues in length. In one embodiment, each H domain in the polypeptide is the same length; in another embodiment, not all H domains in the polypeptide are the same length.

The linkers serve to appropriately space adjacent secondary structure elements. The polypeptides may comprise 2-4, 2-3, 3-4, 2, 3, or 4 linkers. In further embodiments, each linker is independently 2-6, 2-5, 2-4, 2-3, 3-6, 3-5, 3-4, 4-6, 4-5, 5-6, 2, 3, 4, 5, or 6 amino acids in length. In one embodiment, each linker in the polypeptide is the same length; in another embodiment, not all linkers in the polypeptide are the same length. The linkers may be of any suitable amino acid sequence, and each linker in a given polypeptide may be the same or different.

In various further embodiments, the polypeptide is 25-50, 30-50, 35-50, 40-50, 45-50, 25-45, 30-45, 35-45, 40-45, 25-40, 30-40, 35-40, 25-35, 30-35, 25-30, or 40-43 amino acid residues in length.

As used throughout the present application, the term “polypeptide” is used in its broadest sense to refer to a sequence of subunit amino acids. The polypeptides of the invention may comprise glycine, L-amino acids, D-amino acids (which are resistant to L-amino acid-specific proteases in vivo), or a combination of glycine and D- and L-amino acids. As disclosed herein, L-amino acids and glycine are shown in upper case letters, and D-amino acids are shown in lower case letters.

In another embodiment, the polypeptide is at least 30% identical along its entire length to the amino acid sequence of any one of SEQ ID NOS: 1-4000, or a mirror image thereof (i.e.: L amino acids substituted with D amino acids). In various further embodiments, the polypeptide is at least 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to the amino acid sequence of any one of SEQ ID NOS: 1-4000, or a mirror image thereof.

The polypeptides described herein may be chemically synthesized or recombinantly expressed (when the polypeptide is genetically encodable). The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent.

The polypeptides of the invention may include additional residues at the N-terminus, C-terminus, or both that are not present in the polypeptides of the invention; these additional residues are not included in determining the percent identity of the polypeptides of the invention relative to the reference polypeptide.

As shown in the examples that follow, the specific primary amino acid sequence is not a critical determinant of maintaining the structure of the constrained peptide. Thus, the polypeptides disclosed herein may be substituted with conservative or non-conservative substitutions. In one embodiment, changes from the reference polypeptide may be conservative amino acid substitutions. As used herein, “conservative amino acid substitution” means an amino acid substitution that does not alter or substantially alter polypeptide function or other characteristics. In one such embodiment, L amino acids are substituted with other L-amino acids, D amino acids are substituted with other L amino acids, and glycine may be substituted with L or D amino acids, preferably with D amino acids.

In other embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gin (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gin; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gin or into H is; Asp into Glu; Cys into Ser; Gin into Asn; Glu into Asp; Gly into Ala or into Pro; His into Asn or into Gin; lie into Leu or into Val; Leu into Ile or into Val; Lys into Arg, into Gin or into (flu; Met into Leu, into Tyr or into lie; Phe into Met, into leu or into Tyr, Ser into Thr; Thr into Ser; Trp into Tyr; Tyr into Trp; and/or Phe into Val, into Ile or into Leu. In one embodiment, polar residues (DEHKNQRST) are more mutable than the other residues.

As noted above, the polypeptides of the invention may include additional residues at the N-terminus, C-terminus, or both. Such residues may be any residues suitable for an intended use, including but not limited to detection tags (i.e.: fluorescent proteins, antibody epitope tags, etc.), adaptors, ligands suitable for purposes of purification (His tags, etc.), and peptide domains that add functionality to the polypeptides.

In a further aspect, the present invention provides isolated nucleic acids encoding a polypeptide of the present invention that can be genetically encoded. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present invention provides recombinant expression vectors comprising the isolated nucleic acid of any aspect of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In a further aspect, the present invention provides host cells that comprise the recombinant expression vectors disclosed herein, wherein the host cells can be either prokaryotic or eukaryotic. The cells can be transiently or stably engineered to incorporate the expression vector of the invention, using standard techniques in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

Also provided are methods, comprising:

(a) using a computing device to construct a library of proteins, wherein the computing device designs a sequence to stabilize the backbone of the protein, and wherein the proteins comprise less than about 50 amino acids;

(b) synthesizing the proteins using next-generation gene synthesis;

(c) expressing the proteins in yeast so that every cell displays many copies of one protein sequence on its surface; and

(d) screening the library of proteins for susceptibility to digestion by protease.

Here we combine computational protein design, next-generation gene synthesis, and a high-throughput protease susceptibility assay to measure folding and stability for over 15,000 de novo designed miniproteins, 1,500 natural proteins, 10,000 point-mutants, and 30,000 negative control sequences, identifying over 2,500 new stable designed proteins in four basic folds. This scale—three orders of magnitude greater than that of previous studies of design or folding-enabled us to systematically examine how sequence determines folding and stability in uncharted protein space. Iteration between design and experiment increased the design success rate from 6% to 47%, produced stable proteins unlike those found in nature for topologies where design was initially unsuccessful, and revealed subtle contributions to stability as designs became increasingly optimized. Our approach achieves the long-standing goal of a tight feedback cycle between computation and experiment, and promises to transform computational protein design into a data-driven science.

In one embodiment, the synthesizing step comprises oligo library synthesis technology capable of parallel synthesis of 10⁴-10⁵ arbitrarily specified DNA sequences long enough to encode the proteins. In another embodiment, in the screening step, cells are incubated with varying concentrations of protease, those displaying resistant proteins are isolated by fluorescence-activated cell sorting (FACS), and the frequencies of each protein at each protease concentration are determined by deep sequencing. In a further embodiment, the method further comprising assigning each protein a stability score, wherein the stability score comprises: the difference between the measured EC₅₀ and the predicted EC₅₀ in the unfolded state of the protein, according to a sequence-based model parameterized using EC₅₀ measurements of scrambled sequences. In another embodiment, a stability score of 1 corresponds to a 10-fold higher EC₅₀ than the predicted EC₅₀ in the unfolded state. In a further embodiment, the library comprises 1,000 to 30,000 proteins.

### Examples

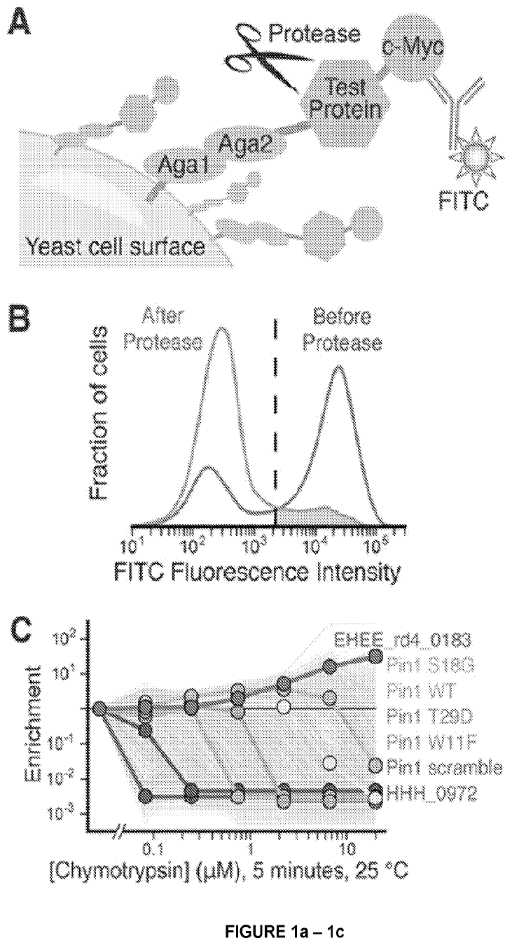

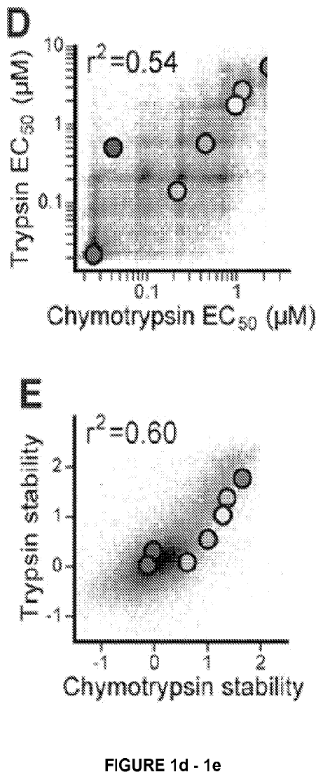

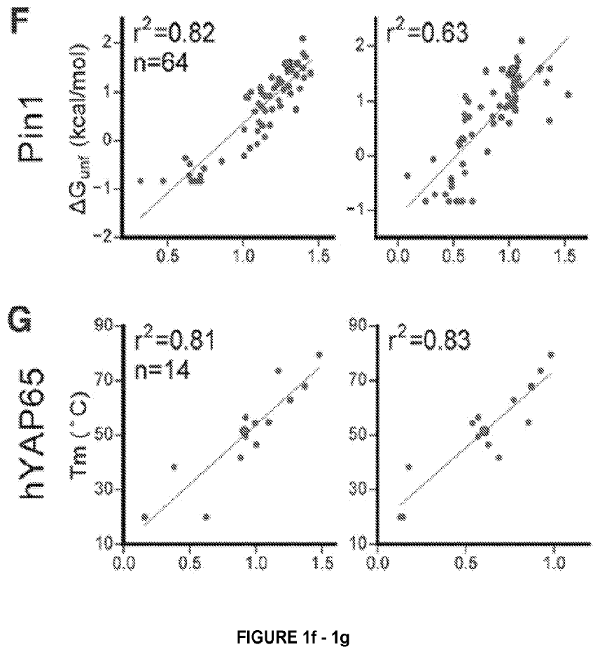

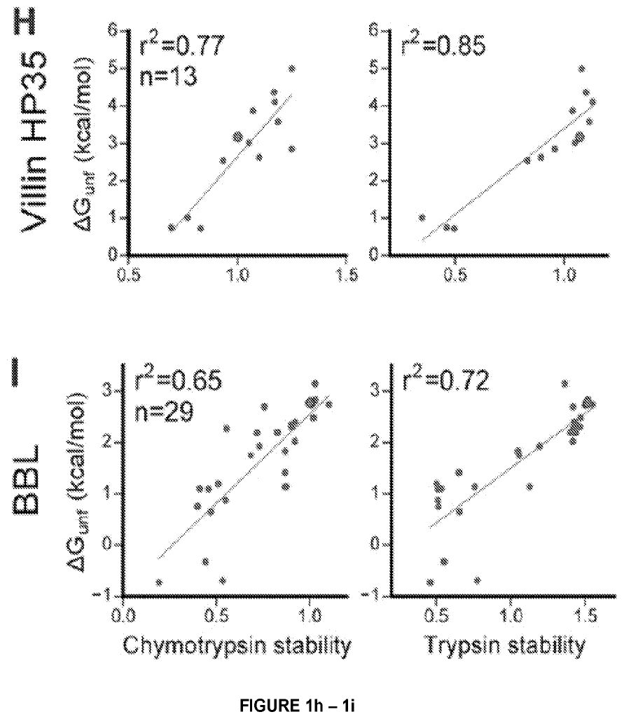

Here we present a new synthetic approach to examine the determinants of protein folding by exploring the space of potential minimal proteins using de novo computational protein design on a three order of magnitude larger scale. To enable this new scale, both DNA synthesis and protein stability measurements are parallelized. To encode our designs, we employ oligo library synthesis technology (22), originally developed for transcriptional profiling and large gene assembly applications, and now capable of parallel synthesis of 10⁴-10⁵ arbitrarily specified DNA sequences long enough to encode short proteins. To assay designs for stability, we expressed these libraries in yeast so that every cell displays many copies of one protein sequence on its surface, genetically fused to an expression tag that can be fluorescently labeled (23)(FIG. 1A). Cells were then incubated with varying concentrations of protease, those displaying resistant proteins were isolated by FACS (FIG. 1B), and the frequencies of each protein at each protease concentration were determined by deep sequencing (FIG. 1C). We then inferred protease EC₅₀ values for each sequence from these data by modeling the complete selection procedure (FIG. 1D, details given in Methods). Finally, each design was assigned a “stability score” (FIG. 1E): the difference between the measured EC₅₀ and the predicted EC₅₀ in the unfolded state, according to a sequence-based model parameterized using EC₅₀ measurements of scrambled sequences. A stability score of 1 corresponds to a 10-fold higher EC₅₀ than the predicted EC₅₀ in the unfolded state. The amino acid sequences of peptides developed herein are provided as SEQ ID NOs: 1-4000 in the accompanying sequence listing.

### Massively Parallel Measurement of Folding Stability

Proteolysis assays have been previously used to measure stability for individual sequences (24) and to select for stable sequences in a proteome (25) or combinatorial library (26, 27), but this approach has not been applied to date to quantify stability for all sequences in a library. To evaluate the ability of the assay to measure stability on a large scale, we obtained a synthetic DNA library encoding four small proteins (pin1 WW-domain (28), hYAP65 WW-domain (5, 10), villin HP35 (7, 11), and BBL (8)) and 116 mutants of these proteins that have been previously characterized thermodynamically using experiments on purified material. The library also contained 19,610 unrelated sequences, and all sequences were assayed for stability simultaneously as described. Although the stability score is not a directly analog of a thermodynamic parameter, stability scores measured with trypsin and separately measured with chymotrypsin were each well-correlated with folding free energies (or melting temperatures) for all four sets of mutants, with r² values ranging from 0.63 to 0.85 (FIG. 1 F-I). Most mutants in this dataset were predicted to have similar unfolded state EC₅₀ values to their parent sequences, so the relative stability scores of the mutants are very similar to their relative EC₅₀ values. In the case of villin assayed with chymotrypsin, the unfolded state model improved the correlation between protease resistance and folding free energy from r²=0.46 (using raw EC₅₀ values) to the reported r²=0.77 by correcting for the effect mutations such as K70M and F51L have on intrinsic chymotrypsin cleavage rates. The mutual agreement between trypsin results, chymotrypsin results, and experiments on purified protein indicate that the assay provides a robust measure of folding stability for small proteins.

### Massively Parallel Testing of Designed Miniproteins

We selected four protein topologies (ααα, βαββ, αββα, and ββαββ) as initial design targets. These topologies have increasing complexity: the ααα topology features only two linkers and exclusively local secondary structure (helices); the ββαββ fold requires four linkers and features a mixed parallel/antiparallel β-sheet bridging the N- and C-termini. Of these topologies, only ααα proteins have been found in nature within the target size range of 40-43 residues; no proteins have been previously designed in any of the four topologies at this size (excluding designed ααα and βαββ proteins stabilized by multiple disulfide linkages (29)). For each topology, we first designed between 5,000 and 40,000 de novo proteins using a blueprint-based approach. Each design has its own unique three-dimensional main chain conformation and its own unique sequence predicted to be near-optimal for that conformation. We then selected 1,000 designs per topology for experimental testing by ranking the designs by a weighted sum of their computed energies and additional filtering terms [see Methods: Protein design]. The median sequence identity between any pair of tested designs of the same topology ranged from 15-35%, and designs were typically no more than 40-65% identical to any other design. For each design, we also included two control sequences in our library: one made by scrambling the order of amino acids in that design (preserving the overall amino acid composition), and a second made by scrambling the order while preserving both the composition and the hydrophobic or polar character at each position. This library comprised 12,459 unique sequences in total: 4,153 designed proteins and 8,306 control sequences. The designed proteins are named using their secondary structure topology (using H for α-helix and E for β-strand), their design round, and a design number.

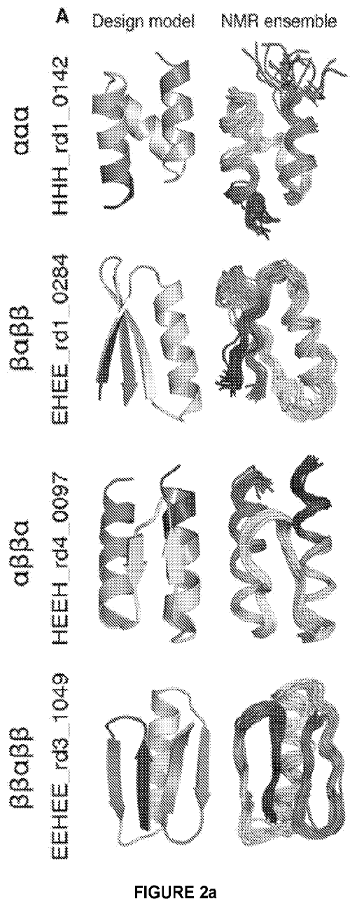

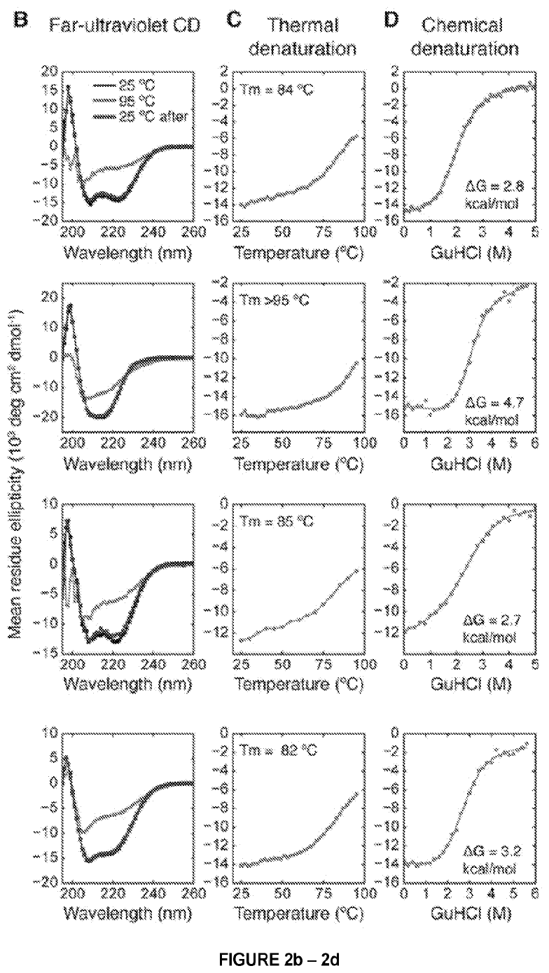

We assayed the sequence library for stability using both chymotrypsin and trypsin. To stringently identify stable designs, we ranked sequences by the lower of their trypsin stability score or their chymotrypsin stability score, referred to simply as their stability score from here on. The fully scrambled sequences and patterned scrambled sequences had similar stability score distributions; most of these controls had stability scores below 0.5, and only one had a score greater than 1.0. In contrast, 206 designed sequences had stability scores above 1.0. Most of these (195/206) were ααα designs (both left-hand and right-handed bundles); the remaining 11 were βαββ. The clustering of the 206 most stable designs around the ααα topology, and the high stability of designed sequences compared with chemically identical control sequences, strongly suggests these stable designs fold into their designed structures. To examine this further, we selected six stable designs (four ααα and two βαββ) for E. coli expression, purification, and further characterization by size-exclusion chromatography (SEC) and circular dichroism spectroscopy (CD). All six designs eluted from SEC as expected for a 5-7 kDa monomer, and the CD spectra were consistent with the designed secondary structure. Five of the six designs had clear, cooperative melting transitions, re-folded reversibly and were highly stable for minimal proteins: all had melting temperatures above 70° C., and the βαββ design EHEE_rd1_0284 (TQTQEFDNEEEARKAEKELRKENRRVTVTQENGRWRVTWD; SEQ ID: 702) had only partially melted at 95° C. (ΔG_{unf}=4.7 kcal/mol, FIG. 2D; the sixth design HHH_rd1_0005 (PDKKKKIVKKLLNKGLDKSEVEKEARKNGISDDIVEQAYKEWL; SEQ ID NO:2669) did not refold and showed signs of aggregation. We determined solution structures for EHEE_rd1_0284 (SEQ ID NO:702) and the left-handed ααα design HHH_rd1_0142 (RKWEEIAERLREEFNINPEEAREAVEKAGiNEEEARRIVKKRL; SEQ ID NO:652) by NMR; each structure closely matched the design model (FIG. 2A. In sum, both high-throughput control experiments and low-throughput characterization of individual proteins indicate that the protease resistant designs fold as designed.

### Global Determinants of Stability

This unprecedentedly large set of stable and unstable minimal proteins with varying physical properties enabled us to quantitatively examine which protein features correlated with folding. We computed over 60 structural and sequence-based metrics and examined which metrics differed between the 195 most stable ααα designs (stability score >1.0, considered to be design successes) and the 664 remaining ααα designs (considered to be failures) using the K-S 2-sample test. Significant differences indicate that a particular metric captures an important contribution to protein stability, and that this contribution was poorly optimized among the tested designs.

The dominant difference between stable and unstable ααα designs was the total amount of buried nonpolar surface area (NPSA) from hydrophobic amino acids. Stable designs buried more NPSA than did unstable designs (p<5e-38), and none of the 95 designs below 32 Å²/residue were stable. Above this threshold, the success rate (successful designs/tested designs) steadily increased as buried NPSA increased. Stable designs also had better agreement between their sequences and their local structures as assessed by quantifying the geometric similarity (in Å of RMSD) between 9-residue long fragments of the designs and 9-residue long fragments of natural proteins similar in local sequence to the designed fragment (see Methods: Fragment analysis). Fragments of stable designs were more geometrically similar to fragments of natural proteins of similar local sequence, while fragments of unstable designs were more geometrically distant from the fragments of natural proteins matching their local sequence (p<2e-26). Other metrics were only weakly correlated with success despite substantial variability among designs, including different measures of amino acid packing density, and the total Rosetta energy itself. Although local sequence-structure agreement and especially buried NPSA are well known to be important for protein stability, it is very challenging to determine the precise strength of these contributions at a global level in the complex balance of all the energies influencing protein structure. Our results directly demonstrate how specific imbalances led to selection of hundreds of unstable designs, and our data and approach provide a completely new route to refining this balance in biophysical modeling.

### Iterative, Data-Driven Protein Design

We sought to use these findings to increase the success rate of protein design, both by changing the design procedure to increase buried NPSA and by re-weighting the metrics used to select designs for testing (see Methods: Protein design). Using the improved design and ranking procedure, we built a second generation of 4,150 designs, along with two control sequences per design: a pattern-preserving scrambled sequence as before (now also preserving Gly and Pro positions), and a second control identical to the designed sequence, but with the most buried side chain (according to the design model) replaced with aspartate. As in Round 1, almost no scrambled sequences had stability scores above 1 (our cutoff defining success) despite the increased hydrophobicity of the scrambled sequences. However, a much greater fraction of second-generation designs proved stable: success for ααα designs improved from 23% to 69%, βαββ designs improved from 1% to 11% successful, and we also obtained 7 stable αββα designs and one stable ββαββ design. These increases demonstrate how iterative, high-throughput protein design can make concrete improvements in design and modeling. Nearly all stable designs were destabilized via the single buried Asp substitution: the median drop in stability score for these designs was 1.1, and only 33 buried Asp controls had stability scores >1.0 compared with 271 designs. This significant destabilization from a single designed substitution provides further large-scale evidence that the stable designs fold into their designed structures. We purified and characterized seven second-generation proteins by SEC and CD, all of which (including three αββα designs and one ββαββ design) were monomeric, displayed their designed secondary structure in CD, were folded cooperatively, and re-folded reversibly after thermal denaturation. Although the αββα and ββαββ designs were only weakly stable, the second-generation βαββ design EHEE_rd2_0005 (TTRYRFTDEEEARRAAKEWARRGYQVHVTQNGTYWEVEVR; SEQ ID NO:35) is, to our knowledge, the most thermostable minimal protein ever found (lacking disulfides or metal coordination): its CD spectrum is essentially unchanged at 95° C., and its Cm is above 5 M GuHCl.

The amount of buried NPSA was the strongest observed determinant of folding stability for second-generation Poop designs, and continued to show correlation with stability for second-generation aa designs. The success rate for ααα designs improved in Round 2 at all levels of buried NPSA, indicating that improving design properties unrelated to buried NPSA (mainly local sequence-structure compatibility) contributed to the increase in success rate along with the increase in NPSA. To improve the stability of the other two topologies, we built a third generation of designs with even greater buried NPSA, at the cost of increased exposure of hydrophobic surface that could hurt solubility. To increase buried NPSA in the ββαββ topology, we expanded the architecture from 41 to 43 residues. This led to a large increase in the ββαββ success rate (˜0% to 13%) and 236 newly discovered stable ββαββ designs. We purified four third-generation designs and found the ββαββ design EEHEE_rd3_1049 (TTVKGDIKVTFDNPEKAKKYAQKLAKIYQLTVHVHGDTIHVK; SEQ ID NO:199) to be very stable (FIG. 2). We solved the solution structure of this design by NMR, revealing that it folds into its designed structure, which is not found in nature at this size range (FIG. 2). Buried NPSA remained the dominant determinant of stability within the tested ββαββ designs. We also observed that a newly improved Rosetta energy function (optimized independently from this work (19) provided significant discrimination between stable and unstable designs, both for the ββαββ topology and for other topologies.

Having accumulated nearly 1,000 examples of stable designs from rounds 1-3, we asked whether more systematic application of this data could be used to select better designs. We designed 2,000-6,000 new proteins per topology (using the improved energy function), and then selected 1,000 designs each for experimental testing by ranking the designs using linear regression, logistic regression, and gradient boosting regression models trained on the structural features and experimental stabilities of the 10,000 designs from Rounds 1-3. Many designs selected for testing were predicted to have a low likelihood of folding, but were included to increase the sequence diversity of tested designs and because better designs could not be found (see Methods: Protein design). Despite this, an even larger fraction of these designs proved stable than before: most notably, the success rate for βαββ designs increased from 17% to 39%, and the success rate for ββαββ designs increased from 13% to 58%. Although the success rate for designing the αββα topology remained low, five purified fourth-generation designs in this topology possessed the highest stability yet observed for the fold by CD. We solved the structure of one of these (HEEH_rd4_0097) (DVEEQIRRLEEVLKKNQPVTWNGiTYTDPNEIKKVIEELRKSM; SEQ ID NO:3222) by NMR and found that it adopts its designed structure in solution (FIG. 2A). Of the models used to rank designs, the logistic regression was most successful, and was very accurate: when designs are binned according to their predicted success probability, the number of success in each bin is very close to that predicted beforehand by the logistic regression. The accuracy of the regression model demonstrates that large-scale analysis of stable and unstable designed proteins can be used to build predictive models of protein stability. The overall increase in success across the four rounds—from 200 stable designs in Round 1 (nearly all in a single topology) to over 1,800 stable designs in Round 4 spread across all four topologies—also demonstrates the power of our massively parallel approach to drive systematic improvement in protein design.

### Sequence Determinants of Stability

We next examined determinants of stability at the individual residue level by constructing a library containing every possible point mutant of 14 designs, as well as every point mutant in three paradigm proteins from decades of folding research: villin HP35, pin1 WW-domain, and hYAP65 WW-domain L30K mutant. This library of 12,834 point mutants is comparable in size to the 12,561 single mutants found in the entire ProTherm™ database (34) and is unbiased toward specific mutations. We assayed this library for stability using trypsin and chymotrypsin, and determined an overall stability effect for each mutation by using the independent results from each protease to maximize the dynamic range of the assay (see Methods: Mutational stability effects). The mutational effects were qualitatively consistent with the designed structures for 13 of 14 designs. As expected, the positions on the designs that were most sensitive to mutation were the core hydrophobic residues, including many alanine residues, which indicates the designed cores are tightly packed. Mutations to surface residues had much smaller effects, highlighting the potential of these proteins as stable scaffolds whose surfaces can be engineered for diverse applications.

To examine the mutability of protein surfaces in greater detail and to probe more subtle contributions to stability, we divided 260 surface positions from 12 of the designs into categories based on secondary structure, and calculated the average stability effect of each amino acid for each category using the ˜5,000 stability measurements at these positions (Methods: Mutational stability effects). We observed specific, though weak, preferences for helices, helix N-caps, the first and last turns of helices, middle strands and edge strands, and linker residues). Amino acids that were favorable for capping helices (Asp, Ser, Thr, and Asn) were unfavorable within helices; these amino acids (except Asn) were as destabilizing as glycine when inside helices. Hydrophobic side chains were stabilizing even when located on the solvent-facing side of a β-sheet, and this effect was stronger at middle strand positions compared with edge strand positions. Most notably, we observed stabilization from charged amino acids on the first and last turns of α-helices when these charges counteract the C-to-N negative-to-positive helical dipole; charges that enhanced the dipole were destabilizing. We isolated this effect by comparing the average stability of each amino acid on first and last helical turns with the average stability of each amino acid at all helical sites (polar sites only in both cases). This effect remained significant in our data even when we restricted the analysis to positions that were Arg or Lys in the original designs to control for any bias in the designed structures favoring original, designed residues compared with mutant residues, although no significant effect was seen for mutations at Glu positions. We had not examined agreement with this dipolar preference during the four rounds of design, and after this observation, we found that the net favorable charge on first and last helical turns (stabilizing charges minus destabilizing charges summed over all helices) discriminated between stable and unstable fourth-generation ααα designs better than any other metric we examined, explaining in part why the success rate had not reached 100%.

In the three naturally occurring proteins, mutations at conserved positions were generally destabilizing, although each natural protein possessed several highly conserved positions that we experimentally determined to be unimportant or deleterious to stability. In villin HP35, these were W64, K70, L75, and F76 (villin HP35 consists of residues 42-76), which are required for villin to bind F-actin. In pint, the highly conserved S16 is deleterious for stability, but directly contacts the phosphate on phosphopeptide ligands of pint (38), highlighting a stability-function trade-off in pin1 discoverable without directly assaying function, the conserved residues H32, T37, and W39 are relatively unimportant for stability, but these residues form the peptide recognition pocket in YAP-family WW-domains. These examples illustrate how our approach enables high-throughput identification of functional residues, even without a functional assay or a protein structure, via comparison between stability data and residue conservation.

### Stability Measurement of all Known Small Protein Domains

How stable are these designed proteins compared with naturally occurring proteins? To examine this, we synthesized DNA encoding (1) all 472 sequences in the protein databank (PDB) between 20 and 50 residues in length and containing only the 19 non-Cys amino acids, and (2) one representative for all 706 domains meeting these criteria in the Pfam protein family database. We included this DNA (and DNA for all stable designs from rounds 1-3) in the library containing our fourth-generation designs to facilitate a head-to-head comparison. Many of the PDB structures are multimeric, and the most resistant overall sequence (measured by stability score) was a C-terminal coiled-coil domain from a TRP channel (3SRO, stability score 1.93). Of the 100 unique, monomeric sequences with PDB structures, the most protease-resistant was a peripheral subunit binding domain (ααα topology) from the thermophile Bacillus stearothermophilus (2PDD, stability score 1.48), which has been heavily studied as an ultrafast-folding protein. A total of 774 designed proteins had higher stability scores than this most protease-resistant natural monomeric protein. The number of stable proteins discovered is orders of magnitude greater than that of natural proteins in the PDB (monomeric or not) in this size range.

### Conclusion

We have shown that proteins can be computationally designed and assayed for folding thousands at a time, and that high-throughput design experiments can provide quantitative insights into the determinants of protein stability. Large libraries can be designed in a relatively unbiased manner (as in our first generation) to maximize the protein property space examined, or properties can be tuned to increase the design success rate at the cost of diversity. The power of our iterative learning approach to progressively hone in on more subtle contributions to stability is highlighted by the progression of our ααα design sets from early rounds in which design failures were caused by insufficient buried nonpolar surface area to the last round where helix-sidechain electrostatics had the greater effect. The large numbers of folded and not folded designs will also provide stringent tests of molecular dynamics simulation approaches which have successfully reproduced structures and some thermodynamic measurements of natural proteins, but have not yet been challenged with plausible but unstable protein structures like our design failures.

The four solution structures, saturation mutagenesis data on 13 of 14 designs, and over thirty thousand negative control experiments indicate that the large majority of our stable sequences are structured as designed. These thousands of designed proteins, stable without disulfides or metal coordination, should have numerous applications in biotechnology and synthetic biology. Many are more stable than any comparably-sized monomeric proteins found in the PDB, making them ideal scaffolds for engineering inhibitors of intracellular protein-protein interactions. Their small size may also help promote membrane translocation and endosomal escape. As DNA synthesis technology continues to improve, high-throughput protein design will become possible for larger proteins as well, revealing determinants of protein stability in more complex structures and leading to a new era of iterative, data-driven de novo protein design and modeling.

### Methods

### Protein Design

All protein design in this work was performed in three stages: (1) backbone construction, (2) sequence design, and (3) selection of designs for testing. Backbone construction (the de novo creation of a compact, three-dimensional backbone with a pre-specified secondary structure) was performed using a blueprint-based approach described previously (34, 34). Briefly, blueprint files were built by hand for each topology in order to define (a) the secondary structure at each residue position for that topology, and (b) the strand pairing and register of any β-sheets. These blueprint files were then used to select short three-dimensional fragments from protein crystal structures matching the proposed secondary structure in the blueprint (200 fragments for every 3- and 9-residues-length stretch of the blueprint). Finally, these fragments were assembled into a full protein backbone using Monte Carlo sampling with a coarse-grained energy function (including constraints on the hydrogen-bonding pairs of residues as specified for the β-sheets in the blueprint) until the overall backbone matched the specified secondary structure and topology, satisfied compactness criteria, and avoided steric clashes. The same four HHH blueprints (each 43 residues in length) were used for all four design rounds. One 40-residue-length EHEE blueprint was used for all four design rounds. A total of 42, 12, 27, and 7 HEEH blueprints (each 43 residues in length) were used in design rounds 1-4 respectively. Blueprints for each round were selected based on the stabilities of designs from the prior round; new blueprints were also introduced in design rounds 2 and 3. A total of 2, 1, 4, and 7 EEHEE blueprints were used in design rounds 1-4 respectively. New blueprints were introduced in design round 3 that increased the protein length from 41 residues to 42 or 43 residues in order to increase the size of the potential hydrophobic core and increase the helix length (blueprints for design rounds 1-4 were 41, 41, 41/42/43, and 43 residues long respectively).

Each backbone structure produced above was used as the input to the Rosetta™ sequence design protocol FastDesign™, also described previously (33). This protocol alternates between (a) a fixed-backbone Monte Carlo search in sequence and rotamer space, and (b) a fixed-sequence backbone relaxation step. This protocol begins with a softened repulsive potential and restores this potential to full strength across several cycles of design and relaxation. These design steps employ the Rosetta full-atom energy function. Design rounds and 1 and 2 employed the Talaris™2013 version of the energy function (40): design round 3 employed the beta_july15 version of the energy function, and design round 4 employed the beta_nov15 version of the energy function (19, 55). The allowed amino acids at each position were restricted using the LayerDesign™ protocol (34); these restrictions are imposed separately from the design energy function for more efficient sampling and to account for design criteria not reflected in the energy function, such as solubility. In this protocol, positions on the designed structure are classified into “core”, “boundary”, and “surface” layers according to their degree of burial, and polar amino acids are excluded from positions in the core layer while nonpolar amino acids are excluded from positions in the surface layer. Layer classification was performed using the “sidechain neighbors” protocol, which counts the number of neighboring residues in the region around the side chain of a given residue. Layer classification is performed on each structure individually and can change during the design process as the structure changes. The definitions of each layer (e.g. the level of burial required for a residue to be classified as core or boundary) were adjusted from design round to design round in order to increase the number of positions where hydrophobic amino acids were permitted. The specific layer definitions used at each stage are given in the included design scripts. For finer control over hydrophobic and polar positioning for the Round 4 designs, we manually specified the allowed amino acids at each position in the designed topologies using resfiles. Starting from design round 3, we included an amino acid composition-based energy term in the design energy function that penalized sequences possessing too few nonpolar amino acids. Finally, to limit the number of designs analyzed at the final selection stage, designs were filtered following sequence design using several basic criteria (primarily compactness and overall score).

After designing 2,000-40,000 finished designs per topology, we then analyzed and ranked these across diverse structural metrics inside and outside of Rosetta™ in order to select the final set of designs for experimental testing. In design rounds 1-3, this ranking was performed by selecting metrics of interest and assigning weights to each metric (again by hand) in order to produce a single composite design score, which was the sum of each metric multiplied by its weight. The selected metrics and their weights were adjusted until the top-ranking designs appeared optimal to the designer. Only a small number of metrics were used in design round 1 in order to ensure broad sampling of protein properties; additional metrics were added in further design cycles as new causes of failure were identified. Different weights were used for each different topology. All scoring metrics used are defined in the section Methods: Definition of scoring metrics.

The metrics used for ranking designs in Round 1 evaluated each design's overall energy (total_score), β-sheet quality (hbondr_lr_bb_per_res), packing (cavity volume, degree, holes, AlaCount, pack), hydrophobic burial (buried_np, one_core_each, two_core_each, percent_core_SCN), agreement between sequence and local structure (mismatch_probability), solubility (exposed_hydrophobics), and hydrogen bond satisfaction (unsat_hbond). Based on the metrics that correlated with design success in Round 1 (as described in the text), we adjusted these weights to select new designs for Round 2, and also added additional metrics related to nonpolar burial (buried_minus_exposed, buried_over_exposed, contact_all) and the geometric similarity between 9-residue-long fragments of the designs and fragments of natural proteins of similar local sequence (avg_best_frag, worst6frags, worstfrag). We again adjusted these weights to select designs for Round 3, and added additional measures of local sequence-structure compatibility (abego_res_profile, p_aa_pp), fragment quality (avg_all_frags), packing (fa_atr_per_res, ss_sc), nonpolar burial (n_hphob_clusters, largest_hphob_cluster, hphob_sc_contacts), the spacing between nonpolar amino acids along the primary sequence (contig_not_hp_avg, contig_not_hp_max), and solubility (fxn_exposed_is_np). In design round 3, we also introduced restrictions on the sequence similarity of the selected βαββ designs (this topology featured the lowest amount of sequence variation between designs): rather than selecting the highest-ranking designs for testing, we selected designs in rank order while passing over designs that were more than 60% identical to a higher-ranking design already selected for testing.

For design round 4, we employed an automated design ranking scheme. All designs from all rounds were scored with ˜50 structural metrics, and the structural metrics and experimental stability scores of the Round 1-3 designs were used to fit topology-specific linear regression, logistic regression, and gradient boosting regressions to predict experimental outcome (stability score) as a function of the design structural metrics. Models were fit using the scikit.learn package (56). Logistic regressions employed Li-regularization with C=0.1. Gradient boosting regressions employed 250 estimators with a tree depth of 5, the minimum samples per split set to 5, a learning rate of 0.01, and a least-squares loss function. All potential Round 4 designs were then ranked according to their predicted stabilities (linear and gradient boosting regression) or success probabilities (logistic regression), and designs were selected for testing in order of the lowest rank given to each design by any of the three regression models. In selecting designs for testing, we again passed over designs that were too similar to designs already selected for testing. We used a threshold of 70% identity for ααα and βαββ designs, and a threshold of 75% ββαββ designs (this was unnecessary for αββα designs). The median identity between designs sharing a topology remained 20-50%.

### DNA Synthesis

All sequences were reverse translated and codon optimized using DNAworks2.0 (57). Sequences were optimized using E. coli codon frequencies despite being used for expression in yeast. Oligo libraries encoding designs and control sequences for design rounds 1 and 2 (12,472 sequences per round) were purchased from CustomArray™, Inc. The oligo library for design round 3 (12,524 sequences) was purchased from Twist Bioscience. Oligo libraries for the point mutant library (13,564 sequences) and design round 4 (18,527 sequences, including the natural protein sequences) were ordered from Agilent Technologies in 27,000 feature format and selectively amplified out of the 27,000-sequence pool in the initial qPCR step. In order to amplify all sequences in a library as evenly as possible, we padded all sequences with extra residues until the amplified region of every oligo had the same length. All libraries were 43 residues in length (not including 18 bp adapter sequences on both ends), except design round 4 where all sequences were 50 residues in length. The saturation mutagenesis library also included 46-residue length oligos for the hYAP65 sequences. In the 43-residue libraries, the shorter EHEE and EEHEE sequences were padded with GSS or GS at the N-terminus. In design library 4, EHEE and EEHEE designed sequenced were again padded at the N-terminus with GSS or GS to reach 43 residues, and then all sequences were padded with N, G, and S residues at the C-terminus so that all sequences would be a uniform 50 residues in length.

### DNA Preparation and Sequencing

Oligo libraries were amplified for yeast transformation in two qPCR steps. First, a 10 ng (CustomArray™ libraries) or 2.5 ng (Twist and Agilent libraries) quantity of synthetic DNA was amplified in a 25 μL reaction using Kapa HiFi™ Polymerase (Kapa Biosystems) for 10-20 cycles by qPCR. The number of cycles was chosen based on a test qPCR run in order to terminate the reaction at 50% maximum yield and avoid overamplification. Second, this reaction product was gel extracted to isolate the expected length product, and re-amplified by qPCR as before to generate sufficient DNA for high-efficiency yeast transformation. Each second qPCR reaction used 1/25^{th} of the gel extraction product as template. This second PCR product was PCR purified and concentrated for transformation of EBY100 yeast using the protocol of (58)(3 μg of insert and 1.5 μg of cut vector per transformation). Yeast display employed a modified version of the pETcon™ vector (59) (known as “pETcon 3”), altered to remove a long single-nucleotide stretch near the cloning region. The amplified libraries included 40 bp segments on either end to enable homologous recombination with the pETcon vector. Gel extraction and PCR purification were performed using QIAquick™ kits (Qiagen Inc).

DNA libraries for deep sequencing were prepared as above, except the first step started from yeast plasmid prepared from 5×10⁷ to 1×10⁸ cells by Zymoprep™ (Zymo Research). Cells were frozen at −80° C. before and after the zymolase digestion step to promote efficient lysis. One-half the plasmid yield from the Zymoprep™ was used as the template for the first PCR amplification. Illumina adapters and 6-bp pool-specific barcodes were added in the second qPCR step. Unlike libraries prepared for transformation, DNA prepared for deep sequencing was gel extracted following the second amplification step. All libraries before and after selections were sequenced using Illumina NextSeq™ sequencing.

### Yeast Display Proteolysis

S. cerevisiae (strain EBY100) cultures were grown and induced as in (26). Following induction, cell density (O.D.₆₀₀) was measured by NanoDrop™, and an amount of cells corresponding to 1 mL at O.D. 1 (12-15M cells) was added to each microcentrifuge tube for proteolysis. Cells were washed and resuspended in 250 μL buffer (20 mM NaPi 150 mM NaCl pH 7.4 (PBS) for trypsin reactions, or 20 mM Tris 100 mM NaCl pH 8.0 (TBS) for chymotrypsin reactions). Proteolysis was initiated by adding 250 μL of room temperature protease in buffer (PBS or TBS) followed by vortexing and incubating the reaction at room temperature (proteolysis reactions took place at cell O.D. 2). After 5 minutes, the reaction was quenched by adding 1 mL of chilled buffer containing 1% BSA (referred to as PBSF or TBSF), and cells were immediately washed 4× in chilled PBSF or TBSF. Cells were then labeled with anti-c-Myc-FITC for 10 minutes, washed twice with chilled PBSF, and then sorted using a Sony SH800 flow cytometer using “Ultra Purity” settings. Events were initially gated by forward scattering area and back scattering area to collect the main yeast population, and then by forward scattering width and forward scattering height to separate individual and dividing cells (which were used for analysis) from cell clumps (which were discarded). Following these gates, cells were gated by fluorescence intensity in one-dimension (FIG. 1B), with the threshold separating displaying (fluorescent) from non-displaying (non-fluorescent) cells set at ˜2,200 fluorescence units (FIG. 1B). Small adjustments were made to this gate based on daily conditions to maximize the separation between the major displaying and non-displaying populations. For each sort, we recorded the fraction of cells passing the fluorescence threshold before proteolysis (using cells from the same starting yeast population, but untreated with protease) and after proteolysis, and also recorded the total number of cells collected for each condition.

Design libraries 1-4 were assayed at six protease concentrations over three sequential selection rounds. Trypsin assays used 0.07 μM, 0.21 μM, 0.64 μM, 1.93 μM, 5.78 μM, and 17.33 μM protease; chymotrypsin assays used 0.08 μM, 0.25 μM, 0.74 μM, 2.22 μM, 6.67 μM, and 20.00 μM protease. Selections using the lowest two concentrations of each protease (0.07 μM and 0.21 μM trypsin and 0.08 μM and 0.25 μM chymotrypsin) were performed starting from the naïve yeast library. The middle two selections (0.64 μM and 1.93 μM trypsin and 0.74 μM and 2.22 μM chymotrypsin) were performed starting from the post-selection 0.21 μM trypsin or 0.25 μM chymotrypsin cultures after 12-24 hours of growth and 12-24 hours of fresh induction. The highest concentration selections were performed starting from the post-selection 1.93 μM trypsin or 2.22 μM chymotrypsin cultures again following growth and re-induction.

The saturation mutagenesis library was assayed at six (trypsin) or eight (chymotrypsin) protease concentrations over four sequential selection rounds. Trypsin assays used 0.41 M, 0.81 μM, 1.63 μM, 3.25 μM, 6.50 μM, and 13.00 μM protease; chymotrypsin assays used 0.21 μM, 0.42 μM, 0.84 μM, 1.69 μM, 3.38 μM, 6.75 μM, 13.50 μM, and 27.00 μM protease. As before, selections 1 and 2 were performed starting from the naïve library, selections 3 and 4 were performed starting from the selection 2 culture following growth and re-induction, selections 5 and 6 were performed starting from the selection 4 culture following growth and re-induction, and selections 7 and 8 (only done for chymotrypsin) were performed starting from the selection 6 culture following growth and re-induction. For trypsin, selection 6 was performed starting from the selection 5 culture following growth and re-induction.

### Protease Reagents

Trypsin-EDTA (0.25%) solution was purchased from Life Technologies and stored at stock concentration (2.5 mg/mL) at −20° C. α-Chymotrypsin from bovine pancreas was purchased from Sigma-Aldrich as lyophilized powder and stored at 1 mg/mL in TBS+100 mM CaCl₂ at −20° C. Each reaction used a freshly thawed aliquot of protease. The trypsin stock activity was measured to be 5,410±312 BAEE units (ΔA₂₅₃×1,000/l minute) per mg in PBS buffer, pH 7.4, with 0.23 mM BAEE (Sigma-Aldrich). Using the Pierce Fluorescent Protease Assay Kit with the fluorescence protocol (ThermoFisher Scientific), 1 mg of the chymotrypsin stock was measured to have equivalent activity to 3.74*0.31 mg of the trypsin stock in pH 7.4 PBS buffer at 25° C.

### Processing of Raw Deep Sequencing Data

Each library in a sequencing run was identified via a unique 6 bp barcode. Following sequencing, reads were paired using the PEAR™ program (60). Reads were considered counts for a particular ordered sequence if the read (1) contained the complete NdeI cut site sequence immediately upstream from the ordered sequence, (2) contained the complete XhoI cut site sequence immediately downstream from the ordered sequence, and (3) matched the ordered sequence at the amino acid level (for sequences in designed libraries 1-4) or at the nucleotide level (sequences in the saturation mutagenesis library). A higher stringency was used for the saturation mutagenesis library due to the overall similarity of the sequences in the library.

EC₅₀ Estimation from Sequencing Counts

To determine protease resistance from our raw sequencing data we built a probabilistic model of the cleavage and selection procedure and used this model to calculate maximum a posteriori estimates of the protease EC₅₀ of each member of the pool. To build the model, we assumed that proteolysis (i.e. any cleavage that results in detachment of the epitope tag) follows pseudo-first order kinetics, with a rate constant specific to each sequence. The fraction of surviving, tagged surface proteins for a given sequence after proteolysis is therefore:

fₛₚᵣₒₜ=e^{-k}^{ₚ}^{[E]t} (1)

where kₚ is a sequence-specific rate constant, [E] is the concentration of protease and t is the reaction time.

In the assay, each cell has a labeling intensity proportional to the number of displayed proteins on its surface. Within the expressing population of cells, we assumed that the number of displayed proteins per cell is log-normally distributed, resulting a distribution of labeling intensities L_{cell} ∝ ln N(μ, σ²) with sequence-independent expression location and scale parameters β and σ. The fraction of cells collected at the labeling threshold L_{cell}>Lₛ is then given by the cumulative distribution function:

$$\begin{matrix}{Frac}_{sel} = 1 - {CDF}_{lognormal} ( {L}_{s} , μ , σ ) & ( 2 ) \\ = \frac{1}{2} - \frac{1}{2} erf [ \frac{ln {L}_{s} - μ}{\sqrt{2} σ} ] & ( 3 )\end{matrix}$$

Following proteolysis, labeling intensity is given by Lₚₒₛₜ=L_{cell}*fₛₚᵣₒₜ and cells are collected when Lₚₒₛₜ>Lₛ. With a fixed selection level Lₛ (defined as e^{c}^{ₛ} in terms of log-intensity rather than absolute intensity) across selection rounds, the fraction of cells collected after proteolysis in each round is given by:

$$\begin{matrix}{Frac}_{sel} = 1 - {CDF}_{lognormal} ( \frac{{L}_{s}}{{f}_{sprot}} , μ , σ ) & ( 4 ) \\ = \frac{1}{2} - \frac{1}{2} erf [ \frac{ln \frac{{L}_{s}}{{f}_{sprot}} - μ}{\sqrt{2} σ} ] \vert {L}_{s} = {e}^{{c}_{s}} & ( 5 ) \\ = \frac{1}{2} - \frac{1}{2} erf [ \frac{{c}_{s} + {k}_{p} [ E ] t - μ}{\sqrt{2} σ} ] & ( 6 )\end{matrix}$$

For model fitting it is advantageous to describe protease stability in terms of a sequence-dependent variable EC₅₀ and a sequence-independent variable Kₛₑₗ. The EC₃₀ for each sequence is defined as the protease concentration at which half of all cells displaying that sequence pass selection. Kₛₑₗ is a constant term representing expression and collection conditions. Setting Fracₛₑₗ=½ allows us to define kₚt in terms of the sequence-specific EC₅₀:

$${k}_{p} t = \frac{μ - {c}_{s}}{{EC}_{50}} \qquad ( 7 )$$

Substituting (7) into (6) and grouping the sequence-independent terms β, σ, and cₛ into Kₛₑₗ yields the form of the model used for fitting:

Frac_{sel}& {\frac{1}{2}}−{\frac{1}{2}}\,\mathrm{erf}{\Big[}{\frac

{c_s+[E]\frac{\mu−c_s}{EC_{50}}−\mu}{{sqrt{2}}\sigma}}{\Big]}\\ (8)

&={\frac{1}{2}}−{\frac{1}{2}}\,\mathrm{erf}{\Big[}{\frac(\mu−

c_s}{\sqrt{2}\sigma}}\left(\frac{[E]}{EC_{50}}−1\right){\Big]}\Big

|K_{sel}=\frac{\mu−c_s}{\sqrt{2}\sigma}\\ (9)

Frac_{sel}(EC_{50},K_{sel},[E])&={\frac{1}{2}}−{\frac

{1}{2}\,\mathrm

{erf}{\Big[}{K_{sel}}\left(\frac{[E]}{EC_{50}}−1\right){\Big]}

\end{aligned}$$ (10)

We modeled each selection experiment as a set of discrete selection events producing both (A) a difference in the observed library population distribution after selection, and (B) a global selection rate during the sorting experiment. For each round of selection with enzyme concentration [E]_{round}, an observed input population distribution Pₘ is updated by a sequence-dependent proteolysis rate to produce an unobserved distribution of labeled cells P_{cleave}:

$&\begin{aligned)}

P_{cleave,i}=\frac{P_{in,i}\times Frac_{sel,i}(EC_{50,i},K_{sel},[

E]_{round})}{\sum{j}\hat{ }{ }

{P_{in,j}\times Frac_{sel,j}(EC_{50,j},K_{sel},[E]_{round})}}

\end{aligned}$$ (11)

for all sequences i, where the denominator of (11) normalizes P_{cleave} so that ΣP_{cleave}=1.

In each selection, n_{assay} total cells are examined, of which the cells nₛₑₗ passing the labeling threshold are collected. The nₛₑₗ collected cells are randomly selected from P_{cleave} during sorting to produce the observed post-selection distribution Pₛₑₗ. To account for carryover during sorting, contamination during library preparation, and sequencing errors that can cause a sequence i to appear in the selected population even when Frac_{sel,i}≈0, we set a lower bound on Fracₛₑₗ equal to 10⁻⁴.

Each library was analyzed by multiple rounds of selection, where the resulting population of a round may be used as the source population for a subsequent selection round at a higher protease concentration (see Methods: Yeast display proteolysis for details). For each round, the observed distribution of matching reads in the pre-selection library is used as Pᵢₙ, which is normalized to sum to 1. The (non-normalized) post-selection distribution Pₛₑₗ (which sums to the total number of cells collected nₛₑₗ) is computed by multiplying nₛₑₗ by the observed normalized distribution of matching reads in the post-selection library. These definitions of Pᵢₙ and Pₛₑₗ assume that there are no sequence-dependent effects in amplification efficiency or sequencing efficiency. If a sequence did not appear in the input distribution Pᵢₙ, any observations of that sequence in the selected population Pₛₑₗ were ignored.

Because the model only considers sequences that match those in the designed library (i.e. only library sequences are included in Pᵢₙ and Pₛₑₗ), the number of cells collected for nₛₑₗ is smaller than the total number of cells collected as reported by the flow cytometer. To account for this, we crudely approximated nₛₑₗ as the number of cells collected by the flow cytometer multiplied by the fraction of sequencing reads that matched sequences in the designed library (i.e. if 200,000 cells were collected in a sort and 75% of sequencing reads for that sort matched library sequences, we assumed in the model that 150,000 cells containing library sequences had been collected). In rare cases where the total number of matching sequencing reads for a given library was smaller than the estimated nₛₑₗ (thus the statistical error was limited by sequencing rather than sorting), the number of matching sequencing reads was used as nₛₑₗ. Finally, to calculate the total number of cells observed (containing library sequences) n_{assay}, we assumed that the overall collection rate (i.e. one collected cell for every twenty observed cells) could be used as a proxy measure of the collection rate for library sequences (as would be true if library sequences dominated the overall cell population, or if the collection rates for library and non-library cells were approximately equal). We calculated the overall collection rate directly from the flow cytometry data as the fraction of initially displaying cells that remained displaying following proteolysis, and then calculated n_{assay} by dividing nₛₑₗ by the overall collection rate.

The complete model log-likelihood is the sum of the data-log likelihoods of Pₛₑₗ and nₛₑₗ and prior likelihoods over the fit parameter EC₅₀, taking Pᵢₙ and n_{assay} as given. Kₛₑₗ was initially treated as a fit parameter as well, but for consistency between all libraries, we fixed Kₛₑₗ at 0.8 for all analysis in this work. The log-likelihood of the observed population Pₛₑₗ was modeled as a multinomial distribution of nₛₑₗ independent selections from P_{cleave}:

Mn(Pₛₑₗ|nₛₑₗ,P_{cleave}) (12)

The log-likelihood of the observed global selection rate was modeled as a binomial distribution of selection events, where the selection probability is the weighted mean of sequence-dependent proteolysis rates:

$$Bn ( {n}_{sel} \vert {n}_{assay} , {Frac}_{sel , pop} ) \vert {Frac}_{sel , pop} = {\sum }_{i} {Frac}_{sel , i} {P}_{in , i} \qquad ( 13 )$$

for all sequences i. Uniform priors covering the range of experimentally relevant values were used for the model parameters. The MAP estimate of the model parameters is found by optimizing the expression:

$${argmax}_{{EC}_{50}} {\sum }_{r ∈ round} {Mn}_{r} + {B}_{{n}_{r}} \qquad ( 14 )$$

The 95% credible intervals for all EC₅₀s were also estimated from the likelihood expression given in (14). All model components were implemented in Python via PyMC3™ (61) and Theano™ (62).

For the analysis of design success rates and design features correlating with success, we excluded sequences whose model-estimated EC₅₀ credible intervals were large. To include as much data as possible, we used a permissive threshold: designs were included in the analysis if their chymotrypsin and trypsin stability score 95% credible intervals (directly taken from the BCs credible intervals) were smaller than 0.95 stability score units (A factor of 9 in [protease]; the equivalent of two rounds of sorting). These thresholds excluded from analysis 14%, 30%, 0.7%, and 1% of sequences from design rounds 1-4 respectively. Credible intervals were much narrower in the later rounds due to improved DNA libraries and better representation of each design in sorting. Despite the permissive thresholds, the median 95% credible interval width for stability scores for sequences included in the analysis was 0.14 stability score units, and 95% of the credible intervals were smaller than 0.48 stability score units.

### Unfolded State Model

We trained a model for the expected protease EC₅₀ of an unfolded protein using our stability data on scrambled sequences. We used both fully scrambled sequences and hydrophobic-polar pattern-preserving scrambled sequences as training data (˜18,000 sequences in total). Only sequences with EC₅₀ value 95% credible intervals smaller than a factor of 3 in [protease] were used for model fitting (protease concentrations increased by this amount at each selection step).

To define the model, we separated the cutting rate into a fixed rate for the constant regions of the fusion construct and an individual rate of cutting at each site i in the inserted sequence. This was done by rearranging equation (7):

$${EC}_{50} = \frac{μ - c}{{k}_{f} t + {\sum }_{i = 1}^{n} {k}_{i} t} \qquad ( 15 )$$

where k_{f} is the pseudo-first order rate constant for the constant regions of the fusion construct in M_{enzyme}⁻¹ s⁻¹, kᵢ is the cleavage rate after amino acid i for all n residues in the inserted sequence (same units), and t is time. If we assume that (1) the inserted sequence cannot affect the cleavage rate of the constant sequence, and (2) that the inserted sequence is completely uncleaved (all kᵢ=0), then the EC₅₀ reaches a maximum that is independent of the inserted sequence:

$${EC}_{50 max} = \frac{μ - c}{{k}_{f} t} \qquad ( 16 )$$

By dividing the numerator and denominator on the right-hand-side of (15) by k_{f}t, we can re-write (15) as:

$${EC}_{50} = \frac{{EC}_{50 max}}{1 + {\sum }_{i = 1}^{n} \frac{{k}_{i}}{{k}_{f}}} \qquad ( 17 )$$

We modeled kᵢ/k_{f} as a function of the 9-residue-long local sequence surrounding sequence position i. In other words, the cut rate at site i in the model depends on the amino acid identities at sites i−4 through i+4, referred to as sites P5 to P4′ in protease nomenclature. The effects of the sequence at these positions are implemented through a position-specific scoring matrix (PSSM) with coefficients for all 19 amino acids (excluding cysteine) at positions P5-P4′ around a potential cut site. The model for kᵢ/k_{f} as a function of the local sequence is given below:

$$\frac{{k}_{i}}{{k}_{f}} = \frac{{k}_{max}}{1 + exp ( {c}_{0} - {\sum }_{site = P 5}^{P 4} PSSM ( {aa}_{site} , site ) )} \qquad ( 18 )$$

where aaₛᵢₜₑ is the amino acid identity at site. The parameters of the full model are EC₅₀ₘₐₓ, kₘₐₓ, c₀, and the 19×9=171 elements of the PSSM. Distributing the c₀ term into the PSSM coefficients would not affect the model (c₀ adds no additional model freedom), but including the term aided model fitting. Positive PSSM coefficients lead to a smaller denominator in eqn. (18), an increased cutting rate kᵢ, and a lower predicted EC₅₀ by eqn. (17). The model parameters (referred to collectively as θ) were trained by minimizing the logarithmic error between the model predicted EC₅₀s and the observed EC₅₀s over the training set of scrambled sequences. We used a combination of squared-error and absolute error in the objective function to provide slightly more tolerance for large outliers than squared-error alone.

$$\text{?} \text{?} {( log ( \text{?} ) - log ( \text{?} ( seq , \text{?} ) ) )}^{2} + 0.25 \times abs ( log ( \text{?} ) - log ( \text{?} ( seq , \text{?} ) ) ) \\ \text{?} \text{indicates text missing or illegible when filed} \qquad ( 19 )$$

We trained the model starting from a uniform PSSM by iterating between fitting only the P1 component of the PSSM, all other positions of the PSSM, and the EC_{50,MAX}, kₘₐₓ, and c₀ terms. We validated the model using three-fold cross validation (three separate models built by excluding a different one-third of the data at a time, followed by predicting each EC₅₀ using the model that did not encounter that sequence during training). The cross-validated root-mean-squared errors (RMSE) for trypsin and chymotrypsin are 2% (trypsin) and 5% (chymotrypsin) higher than the RMSEs of the models trained using all data without cross-validation, indicating minimal overfitting. The predictions made by the cross-validated models are very similar to the predictions made by the models trained on the complete dataset.

All stability scores reported herein were calculated using the final version of the unfolded state model, trained using the scrambled sequence data from all four design rounds. Obviously, data on the full set of scrambled sequences was not available at earlier stages of the work. The data analysis after each design round employed earlier versions of the unfolded state model that were trained using the scrambled sequence data that had been collected up to that point. However, the final model predictions used for the manuscript are very similar to the model predictions made when only the data from Round 1 are used for training.

Because the unfolded state model is trained on EC₅₀s of scrambled sequences and not on designed sequences, a systematic bias may be introduced that would cause scrambled sequences to receive lower stability scores than designed sequences (the stability score is the deviation of each sequence's measured EC₅₀ from the unfolded state model's predicted EC₅₀; if the model were overfit, the sequences used in training would have incorrectly low deviations). However, the cross-validation results indicate that only minimal overfitting is present in the model parameters. To further quantify possible bias in the model parameters, we examined the distribution of predicted unfolded state EC₅₀ values for scrambled sequences (which were used in training) and designed sequences (which were not). We would expect these distributions to be the same because the designed and scrambled sequences are very similar at the sequence level. However, on average, the predicted unfolded state EC₅₀ values for the scrambled sequences are higher than the predicted EC₅ values for the designed sequences, which biases the scrambled sequences to appear less stable, although this effect is small. This bias likely results from overfitting of EC₅₀ values for partially folded scrambled sequences. Overall, the small bias (0.15-0.16 units of stability score on average) between designed sequences and scrambled sequences does not change the conclusion that hundreds of the designs at each stage are many times more stable than the scrambled sequences, often by 0.5-1.0 stability score units or more.

### Protein Expression and Purification

Two different expression vectors were used to purify the designs chosen for biophysical analysis. Most designs were expressed as isolated domains with an additional 21-residue N-terminal sequence containing a His-tag and thrombin cleavage site to aid purification. Genes encoding these designs were obtained from GenScript™ in the pET-28b+ expression vector. The remaining designs were expressed as fusions with the yeast SUMO domain Smt3 using the custom vector pCDB24. Genes encoding these designs were obtained as gBlocks™ from IDT and inserted into the pCDB24 vector via Gibson assembly (63).

All designs were expressed in E. coli B121* (DE3) cells (Invitrogen). Starter cultures were grown overnight at 37° C. in Luria-Bertani (LB) medium overnight with added antibiotic (50 μg/mi carbenicillin for SUMO expression or 30 μg/ml kanamycin for pET-28b+ expression). These overnight cultures were used to inoculate 500 mL of Studier autoinduction media (64) supplemented with antibiotic, and grown overnight. Cells were harvested by centrifugation at 4° C., resuspended in 25 mL lysis buffer (20 mM imidazole in PBS containing DNAse and protease inhibitors), and lysed by sonication or by microfluidizer. PBS buffer contained 20 mM NaPO₄, 150 mM NaCl, pH 7.4. After removal of insoluble material, the lysates were loaded onto nickel affinity gravity columns to purify the designed proteins by immobilized metal-affinity chromatography (IMAC). Designs expressed as fusions to the SUMO domain were then cleaved from the domain using the Yeast SUMO protease UlpI and dialyzed overnight in PBS at 4° C. to remove excess imidazole before a second IMAC step was used to remove the SUMO tag following cleavage.

For expression of ¹³C-¹⁵N-labeled protein for NMR analysis, the plasmids were transformed into the Lemo21 E. coli expression strain (NEB) and plated on M9/glucose plates containing kanamycin to 50 ug/mL and chloramphenicol to 34 ug/mL, grown at 37° C. overnight. For the starter culture, a single colony was inoculated into a 250 mL baffled flask containing 50 mL of Luria-Bertani medium, with kanamycin to 50 ug/mL, chloramphenicol to 34 ug/mL, and grown for approximately 18 hours at 37° C., shaking at 225 rpm. 10 mL of the starter culture was then transferred to a 2 L baffled flask containing 0.5 L of Terrific Broth (Difco), with 25 mM Na₂HPO₄, 25 mM KH₂PO₄, 50 mM NH₄Cl, 5 mM Na₂SO₄ kanamycin to 50 ug/mL, and chloramphenicol to 34 ug/mL. This expression culture was grown at 37° C. to an OD₆₀₀ of approximately 1.0, then removed from the flask and spun at 4000 rpm for 15 minutes to pellet the cells. The Terrific Broth was removed, and the cells were washed briefly with 30 mL of PBS. The cells were then transferred to a new 2 L baffled flask containing 0.5 L of labeled media (25 mM Na₂HPO₄, 25 mM KH₂PO₄, 50 mM ¹⁵NH₄Cl, 5 mM Na₂SO₄, 0.2% (w/v) ¹³C glucose), kanamycin to 50 ug/mL and chloramphenicol to 34 ug/mL. The cells were allowed to recover at 37° C. for 30 minutes, then IPTG (Carbosynth) was added to 1 mM and the temperature was reduced to 20° C. The cells were harvested the following day and purified by IMAC. The labeled NH₄Cl and glucose were obtained from Cambridge Isotopes.

### Size-Exclusion Chromatography

Following IMAC, designs (labeled and unlabeled) were further purified by size-exclusion chromatography on ÄKTAxpress™ (GE Healthcare) using a Superdex™ 75 10/300 GL column (GE Healthcare) in PBS buffer. The monomeric fraction of each run (typically eluting at the 15 mL mark) was collected and immediately analyzed by CD or flash frozen in liquid N₂ for later analysis.

### Circular Dichroism

Far-ultraviolet CD measurements were carried out with an AVIV spectrometer, model 420. Wavelength scans were measured from 260 to 195 nm at 25 and 95° C. Temperature melts monitored dichroism signal at 220 nm in steps of 2° C./minute with 30 s of equilibration time. Wavelength scans and temperature melts were performed using 0.35 mg/ml protein in PBS buffer (20 mM NaPO₄, 150 mM NaCl, pH 7.4) with a 1 mm path-length cuvette. Chemical denaturation experiments with guanidinium hydrochloride (GuHCl) were performed using an automatic titrator with a protein concentration of 0.035 mg/ml and a 1 cm path-length cuvette with stir bar. The GuHCl concentration was determined by refractive index in PBS buffer. The denaturation process monitored dichroism signal at 220 nm in steps of 0.2 M GdmCl with 1 minute mixing time for each step, at 25° C. Protein concentrations were determined by absorbance at 280 nm measured using a NanoDrop™ spectrophotometer (Thermo Scientific) using predicted extinction coefficients (65). Protein concentrations for designs lacking aromatic amino acids were measured by Qubit™ protein assay (ThermoFisher Scientific).

Melting temperatures were determined by first smoothing the data with a Savitsky-Golay filter of order 3, then approximating the smoothed data with a cubic spline to compute derivatives. The reported Tm is the inflection point of the melting curve. Chemical denaturation curves were fitted by nonlinear regression to a two-state unfolding model with six-parameters: the folding free energy, m-value, and linear pre- and post-transition baselines with individual slope and intercepts (66).

### NMR Structure Determination

NMR data acquisition was carried out at 25° C. (HHH_rd1_0142, EHEE_rd1_0284, and EEHEE_rd3_1049) or 15° C. (HEEH_rd4_0097) on Bruker spectrometers operating at 600 or 800 MHz, and equipped with cryogenic probes. All 3D spectra were acquired with non-uniform sampling schemes in the indirect dimensions and were reconstructed by multidimensional decomposition software MDDNMR (67) or (68), interfaced with NMRPipe™ (69). Conventional backbone and NOESY spectra were acquired as described previously (70), and the automated program ABACUS™ (71) was used to aide in the assignment of backbone and sidechain resonances. Initial automated NOE assignments and structure calculations were performed using the noeassign module in CYANA™ 3.0 (72). The best 20 of 100 CYANA™ structures from the final cycle were refined with CNSSOLVE (73) by performing a short restrained molecular dynamics simulation in explicit solvent (74). The NMR structures of the constructs are comprised of the final 20 refined structures.

### Fragment Analysis

To evaluate agreement between sequence and structure for a given designed protein, we used Rosetta's standard fragment generation protocol (17) to select 200 fragments (9-residue length segments) from natural protein crystal structures for each 9-residue-long segment of the designed protein. The fragments were chosen so that their sequence and secondary structure were as similar as possible to the sequence and predicted secondary structure of the designed protein segment (predicted using PSIPRED™ (75)). If these fragments are highly geometrically similar to the designed segment (measured by RMSD), this indicates that the designed sequence preferentially adopts the designed fold even at the local level, because each local sequence segment is commonly found in its designed local structure when found in solved protein structures.

### Mutational Stability Effects

Instead of using the minimum of the trypsin and chymotrypsin stability scores as an overall stability score for sequences in the point mutant library, we took advantage of the hundreds of mutants available for each protein to calibrate the trypsin and chymotrypsin stability scales in relation to each other for each set of mutants (i.e. mutants of the same wild-type protein). For example, mutations in EHEE_rd1_0882 that cause a chymotrypsin stability score change of 1.0 typically cause a trypsin stability score change of 1.2 (i.e. the slope of the best-fit line is 1.2; the r² for the two datasets is 0.77). However, mutations to EEHEE_rd3_0037 that cause a chymotrypsin stability score change of 1.0 cause a much larger trypsin stability score change of 2.6 (r²=0.71). Because each set of mutants had a characteristic slope, we used these slopes to combine the trypsin and chymotrypsin measurements and compute a consensus stability score for each mutant. These consensus stability scores were assigned in four steps. First, we identified the subset of mutants for each wild-type protein with high-confidence EC₅₀ values (i.e. those that were precisely measured and were within the dynamic range of protease concentrations tested). Second, these high-confidence measurements were then used to determine the slope and intercept of the best-fit line between the trypsin and chymotrypsin stability scores for each protein by orthogonal distance regression. Third, we mapped each mutant of a given protein onto the best-fit line for that protein at one of three positions: the nearest point on the fit line, the point on the fit line at an identical x-coordinate (chymotrypsin stability score), or the point on the fit line at an identical y-coordinate (trypsin stability score) Finally, after mapping all points onto the fit line, we used the x-coordinate of the mapped point (the location of that point on the chymotrypsin axis) as the overall consensus stability score for each mutant.

In examining the best-fit lines between the trypsin and chymotrypsin measurements for each mutant set, we observed that mutants whose predicted unfolded state chymotrypsin EC₅₀ values changed significantly from the predicted unfolded state wild-type EC₅₀ value were often outliers in the fit. These outliers suggested that the chymotrypsin unfolded state model was oversensitive to the effects of single amino acid changes, distorting the fits. To improve the estimation of consensus stability scores, we restricted the deviation between each mutant's predicted unfolded state EC₅₀ and the wild-type predicted unfolded state EC₅₀ to a factor of 2.64 in [chymotrypsin] (2^{1,4}). This only affected 1.2% of mutants, and would not have affected any results in FIG. 1 (no mutants in FIG. 1 deviate by this amount from the wild-type predicted unfolded state EC₅₀ value.)

The average stability effects of each amino acid were calculated using the consensus stability scores described above. To compute the average stability effects using the data, we used the average stability score of the A, E, H, I, M, T, and V mutants at each position as the “baseline” stability at each position (i.e. the average stability score of these mutants was used as the zero-point for a new position-specific stability scale). These amino acids were chosen because they included the different types of amino acid physical properties (polar and hydrophobic, large and small) and because these amino acids generally have minimal impact on a sequence's unfolded state predicted EC₅₀ with either trypsin or chymotrypsin. We then computed the stability of each amino acid at each position relative to this baseline. Finally, we averaged these re-zeroed stability scores across all the different protein sites in a given category (i.e. polar helical positions, edge strand positions, etc.) to determine the average stability effect of each amino acid for that category. The re-zeroing procedure for each site did not affect the relative average stabilities of the different amino acids shown in the figure, but it did lower the associated standard error by removing irrelevant variation in overall protein stability from the measurement. The average stability effects were adjusted a final time to set the mean value of all 20 amino acids to zero. Helical positions were considered to be polar if their wild-type (designed) amino acid was D, B, H, K, N, Q, R, S, T, or Y. The first and last helical turns were defined as the first and last three residues of each helix.

### Natural Protein Compilation

Our 1,178 natural proteins were compiled by querying the PDB on Feb. 4, 2016 for all structures containing only protein (no lipid, carbohydrate, or nucleic acid), with 1 chain per asymmetric unit, chain length 20-50aa, and no modified residues. We then manually filtered this list to remove all sequences containing Cys residues. Pfam sequences were collected from the seed database of Pfam 28.0, taking the first representative of all families lacking a Cys residue.

### Conservation Analysis in Naturally Occurring Proteins

Homologous sequences for villin HP35, pin1 WW-domain, and hYAP65 WW-domain were identified using HHblits (76) with an e-value cutoff of 1e-10 and 4 iterations. HHfilter was used to remove sequences that were more than 90% identical or that covered less than 90% of the query sequence (77). Bits of conservation were calculated using WebLogo 3.3 (78).

The secondary structural arrangements of peptides designed are provided below in Table 1; the amino acid sequences are provided in the accompanying sequence listing.

TABLE 1

| SEQ ID NO: | name |
|---|---|
| SEQ ID NO: 1 | HHH_rd2_0058.pdb |
| SEQ ID NO: 2 | EEHEE_rd3_1702.pdb |
| SEQ ID NO: 3 | EHEE_rd4_0009.pdb |
| SEQ ID NO: 4 | HHH_rd2_0139.pdb |
| SEQ ID NO: 5 | EEHEE_rd3_1058.pdb |
| SEQ ID NO. 6 | HHH_rd2_0040.pdb |
| SEQ ID NO: 7 | HHH_rd2_0161.pdb |
| SEQ ID NO: 8 | HHH_rd4_0231.pdb |
| SEQ ID NO: 9 | HHH_rd2_0175.pdb |
| SEQ ID NO: 10 | HHH_rd4_0996.pdb |
| SEQ ID NO: 11 | HHH_rd2_0158.pdb |
| SEQ ID NO: 12 | HHH_rd4_0734.pdb |
| SEQ ID NO: 13 | HHH_rd4_0289.pdb |
| SEQ ID NO: 14 | HHH_rd2_0031.pdb |
| SEQ ID NO: 15 | HHH_rd4_0167.pdb |
| SEQ ID NO: 16 | HHH_rd4_0353.pdb |
| SEQ ID NO: 17 | EHEE_rd3_0133.pdb |
| SEQ ID NO: 18 | HHH_rd4_0291.pdb |
| SEQ ID NO: 19 | HHH_rd2_0114.pdb |
| SEQ ID NO: 20 | HHH_rd4_0047.pdb |
| SEQ ID NO: 21 | HHH_rd4_0383.pdb |
| SEQ ID NO: 22 | HHH_rd1_0056.pdb |
| SEQ ID NO: 23 | HHH_rd2_0046.pdb |
| SEQ ID NO: 24 | HHH_rd2_0085.pdb |
| SEQ ID NO: 25 | HH_rd4_0303.pdb |
| SEQ ID NO: 26 | HHH_rd2_0039.pdb |
| SEQ ID NO: 27 | EEHEE_rd3_1498.pdb |
| SEQ ID NO: 28 | HHH_rd3_0138.pdb |
| SEQ ID NO: 29 | HHH_rd4_0491.pdb |
| SEQ ID NO: 30 | HHH_rd2_0160.pdb |
| SEQ ID NO: 31 | EEHEE_rd4_0094.pdb |
| SEQ ID NO: 32 | HHH_rd4_0416.pdb |
| SEQ ID NO: 33 | HHH_rd4_0508.pdb |
| SEQ ID NO: 34 | EHEE_rd4_0924.pdb |
| SEQ ID NO: 35 | EHEE_rd2_0005.pdb |
| SEQ ID NO: 36 | HHH_rd4_0920.pdb |
| SEQ ID NO: 37 | HHH_rd4_0130.pdb |
| SEQ ID NO: 38 | HHH_rd4_0819.pdb |
| SEQ ID NO: 39 | HHH_rd4_0049.pdb |
| SEQ ID NO: 40 | HHH_rd3_0008.pdb |
| SEQ ID NO: 41 | EHEE_rd4_0640.pdb |
| SEQ ID NO: 42 | HHH_rd2_0195.pdb |
| SEQ ID NO: 43 | HHH_rd4_0858.pdb |
| SEQ ID NO: 44 | HHH_rd4_0171.pdb |
| SEQ ID NO: 45 | HHH_rd1_0244.pdb |
| SEQ ID NO: 46 | HHH_rd4_0276.pdb |
| SEQ ID NO: 47 | HHH_rd4_0340.pdb |
| SEQ ID NO: 48 | HHH_rd2_0096.pdb |
| SEQ ID NO: 49 | HHH_rd2_0045.pdb |
| SEQ ID NO: 50 | HHH_rd4_0122.pdb |
| SEQ ID NO: 51 | EEHEE_rd4_0057.pdb |
| SEQ ID NO: 52 | HHH_rd4_0179.pdb |
| SEQ ID NO: 53 | HHH_rd4_0581.pdb |
| SEQ ID NO: 54 | HHH_rd4_0159.pdb |
| SEQ ID NO: 55 | HHH_rd4_0116.pdb |
| SEQ ID NO: 56 | HHH_rd4_0044.pdb |
| SEQ ID NO: 57 | HHH_rd4_0132.pdb |
| SEQ ID NO: 58 | EEHEE_rd3_0681.pdb |
| SEQ ID NO: 59 | HHH_rd4_0790.pdb |
| SEQ ID NO: 60 | HHH_rd3_0075.pdb |
| SEQ ID NO: 61 | HHH_rd4_0235.pdb |
| SEQ ID NO: 62 | HHH_rd4_0168.pdb |
| SEQ ID NO: 63 | HHH_rd2_0123.pdb |
| SEQ ID NO: 64 | HHH_rd2_0025.pdb |
| SEQ ID NO: 65 | HHH_rd4_0666.pdb |
| SEQ ID NO: 66 | HHH_rd4_0148.pdb |
| SEQ ID NO: 67 | HHH_rd4_0631.pdb |
| SEQ ID NO: 68 | HHH_rd2_0030.pdb |
| SEQ ID NO: 69 | HHH_rd4_0145.pdb |
| SEQ ID NO: 70 | HHH_rd4_0875.pdb |
| SEQ ID NO: 71 | HHH_rd4_0262.pdb |
| SEQ ID NO: 72 | HHH_rd1_0210.pdb |
| SEQ ID NO: 73 | EHEE_rd3_0124.pdb |
| SEQ ID NO: 74 | HHH_rd4_0619.pdb |
| SEQ ID NO: 75 | HHH_rd4_0141.pdb |
| SEQ ID NO: 76 | EEHEE_rd4_0193.pdb |
| SEQ ID NO: 77 | HHH_rd2_0205.pdb |
| SEQ ID NO: 78 | HHH_rd3_0169.pdb |
| SEQ ID NO: 79 | HHH_rd4_0926.pdb |
| SEQ ID NO: 80 | HHH_rd3_0173.pdb |
| SEQ ID NO: 81 | HHH_rd2_0005.pdb |
| SEQ ID NO: 82 | HHH_rd3_0014.pdb |
| SEQ ID NO: 83 | HHH_rd4_0022.pdb |
| SEQ ID NO: 84 | HHH.rd4_0723.pdb |
| SEQ ID NO: 85 | HHH_rd4_0189.pdb |
| SEQ ID NO: 86 | HHH_rd4_0277.pdb |
| SEQ ID NO: 87 | HHH_rd4_0084.pdb |
| SEQ ID NO: 88 | HHH_rd4_0758.pdb |
| SEQ ID NO: 89 | EEHEE_rd4_0216.pdb |
| SEQ ID NO: 90 | HHH_rd3_0048.pdb |
| SEQ ID NO: 91 | HHH_rd4_0859.pdb |
| SEQ ID NO: 92 | HHH_rd4_0325.pdb |
| SEQ ID NO: 93 | EEHEE_rd4_0159.pdb |
| SEQ ID NO: 94 | EHEE_rd2_0008.pdb |
| SEQ ID NO: 95 | EEHEE_rd4_0002.pdb |
| SEQ ID NO: 96 | HHH_rd4_0260.pdb |
| SEQ ID NO: 97 | HHH_rd4_0549.pdb |
| SEQ ID NO: 98 | HHH_rd4_0177.pdb |
| SEQ ID NO: 99 | HHH_rd3_0085.pdb |
| SEQ ID NO: 100 | HHH_rd4_0001.pdb |
| SEQ ID NO: 101 | HHH_rd4_0836.pdb |
| SEQ ID NO: 102 | HHH_rd4_0237.pdb |
| SEQ ID NO: 103 | EEHEE_rd3_0095.pdb |
| SEQ ID NO: 104 | HHH_rd4_0928.pdb |
| SEQ ID NO: 105 | HHH_rd1_0061.pdb |
| SEQ ID NO: 106 | HHH_rd4_0088.pdb |
| SEQ ID NO: 107 | HHH_rd4_0027.pdb |
| SEQ ID NO: 108 | HHH_rd4_0580.pdb |
| SEQ ID NO: 109 | EHEE_rd2_0331.pdb |
| SEQ ID NO: 110 | HHH_rd2_0143.pdb |
| SEQ ID NO: 111 | EHEE_rd4_0079.pdb |
| SEQ ID NO: 112 | HHH_rd4_0853.pdb |
| SEQ ID NO: 113 | HHH_rd4_0395.pdb |
| SEQ ID NO: 114 | HHH_rd4_0211.pdb |
| SEQ ID NO: 115 | HHH_rd3_0215.pdb |
| SEQ ID NO: 116 | HHH_rd4_0579.pdb |
| SEQ ID NO: 117 | EHEE_rd2_0840.pdb |
| SEQ ID NO: 118 | HHH_rd4_0067.pdb |
| SEQ ID NO: 119 | HHH_rd4_0833.pdb |
| SEQ ID NO: 120 | HHH_rd4_0228.pdb |
| SEQ ID NO: 121 | EEHEE_rd4_0022.pdb |
| SEQ ID NO: 122 | HHH_rd4_0134.pdb |
| SEQ ID NO: 123 | HHH_rd4_0013.pdb |
| SEQ ID NO: 124 | HHH_rd4_0175.pdb |
| SEQ ID NO: 125 | HHH_rd1_0087.pdb |
| SEQ ID NO: 126 | EEHEE_rd3_1136.pdb |
| SEQ ID NO: 127 | HHH_rd4_0709.pdb |
| SEQ ID NO: 128 | HHH_rd2_0074.pdb |
| SEQ ID NO: 129 | HHH_rd1_0579.pdb |
| SEQ ID NO: 130 | EEHEE_rd4_0484.pdb |
| SEQ ID NO: 131 | HHH_rd4_0468.pdb |
| SEQ ID NO: 132 | HHH_rd4_0492.pdb |
| SEQ ID NO: 133 | HHH_rd4_0552.pdb |
| SEQ ID NO: 134 | HHH_rd4_0616.pdb |
| SEQ ID NO: 135 | HHH_rd3_0199.pdb |
| SEQ ID NO: 136 | HHH_rd4_0467.pdb |
| SEQ ID NO: 137 | EEHEE_rd4_0097.pdb |
| SEQ ID NO: 138 | HHH_rd1_0537.pdb |
| SEQ ID NO: 139 | HHH_rd2_0023.pdb |
| SEQ ID NO: 140 | HHH_rd4_0456.pdb |
| SEQ ID NO: 141 | HHH_rd4_0050.pdb |
| SEQ ID NO: 142 | HHH_rd4_0100.pdb |
| SEQ ID NO: 143 | HHH_rd4_0992.pdb |
| SEQ ID NO: 144 | HHH_rd4_0673.pdb |
| SEQ ID NO: 145 | EEHEE_rd3_1817.pdb |
| SEQ ID NO: 146 | HHH_rd3_0107.pdb |
| SEQ ID NO: 147 | HHH_rd4_0682.pdb |
| SEQ ID NO: 148 | HHH_rd2_0063.pdb |
| SEQ ID NO: 149 | HHH_rd2_0032.pdb |
| SEQ ID NO: 150 | HHH_rd4_0003.pdb |
| SEQ ID NO: 151 | EEHEE_rd4_0060.pdb |
| SEQ ID NO: 152 | HHH_rd4_0373.pdb |
| SEQ ID NO: 153 | HHH_rd4_0575.pdb |
| SEQ ID NO: 154 | HHH_rd4_0012.pdb |
| SEQ ID NO: 155 | HHH_rd3_0091.pdb |
| SEQ ID NO: 156 | EHEE_rd4_0196.pdb |
| SEQ ID NO: 157 | EHEE_rd4_0096.pdb |
| SEQ ID NO: 158 | HHH_rd2_0166.pdb |
| SEQ ID NO: 159 | HHH_rd2_0013.pdb |
| SEQ ID NO: 160 | HHH_rd4_0849.pdb |
| SEQ ID NO: 161 | EEHEE_rd4_0005.pdb |
| SEQ ID NO: 162 | HHH_rd4_0728.pdb |
| SEQ ID NO: 163 | HHH_rd4_0804.pdb |
| SEQ ID NO: 164 | HHH_rd4_0869.pdb |
| SEQ ID NO: 165 | HHH_rd4_0745.pdb |
| SEQ ID NO: 166 | HHH_rd4_0934.pdb |
| SEQ ID NO: 167 | EHEE_rd4_0653.pdb |
| SEQ ID NO: 168 | HHH_rd3_0231.pdb |
| SEQ ID NO: 169 | HHH_rd3_0004.pdb |
| SEQ ID NO: 170 | HHH_rd4_0188.pdb |
| SEQ ID NO: 171 | HHH_rd3_0022.pdb |
| SEQ ID NO: 172 | HHH_rd4_0162.pdb |
| SEQ ID NO: 173 | HHH_rd4_0640.pdb |
| SEQ ID NO: 174 | HHH_rd4_0684.pdb |
| SEQ ID NO: 175 | HHH_rd2_0201.pdb |
| SEQ ID NO: 176 | EEHEE_rd3_1220.pdb |
| SEQ ID NO: 177 | EEHEE_rd4_0430.pdb |
| SEQ ID NO: 178 | HHH_rd2_0218.pdb |
| SEQ ID NO: 179 | HHH_rd1_0071.pdb |
| SEQ ID NO: 180 | EHEE_rd4_0130.pdb |
| SEQ ID NO: 181 | HHH_rd4_0612.pdb |
| SEQ ID NO: 182 | HHH_rd4_0808.pdb |
| SEQ ID NO: 183 | EHEE_rd4_0213.pdb |
| SEQ ID NO: 184 | HHH_rd4_0017.pdb |
| SEQ ID NO: 185 | HHH_rd4_0779.pdb |
| SEQ ID NO: 186 | HHH_rd2_0094.pdb |
| SEQ ID NO: 187 | EHEE_rd2_1044.pdb |
| SEQ ID NO: 188 | HHH_rd4_0498.pdb |
| SEQ ID NO: 189 | HHH_rd4_0151.pdb |
| SEQ ID NO: 190 | HHH_rd4_0374.pdb |
| SEQ ID NO: 191 | HHH_rd4_0834.pdb |
| SEQ ID NO: 192 | HHH_rd3_0029.pdb |
| SEQ ID NO: 193 | HHH_rd4_0396.pdb |
| SEQ ID NO: 194 | HHH_rd4_0052.pdb |
| SEQ ID NO: 195 | EHEE_rd2_1260.pdb |
| SEQ ID NO: 196 | EHEE_rd4_0726.pdb |
| SEQ ID NO: 197 | HHH_rd3_0165.pdb |
| SEQ ID NO: 198 | HHH_rd3_0005.pdb |
| SEQ ID NO: 199 | EEHEE_rd3_1049.pdb |
| SEQ ID NO: 200 | HHH_rd4_0528.pdb |
| SEQ ID NO: 201 | EHEE_rd4_0510.pdb |
| SEQ ID NO: 202 | EEHEE_rd4_0029.pdb |
| SEQ ID NO: 203 | HHH_rd4_0752.pdb |
| SEQ ID NO: 204 | HHH_rd4_0722.pdb |
| SEQ ID NO: 205 | HHH_rd4_0028.pdb |
| SEQ ID NO: 206 | EEHEE_rd4_0759.pdb |
| SEQ ID NO: 207 | HHH_rd4_0452.pdb |
| SEQ ID NO: 208 | EEHEE_rd4_0026.pdb |
| SEQ ID NO: 209 | HHH_rd4_0056.pdb |
| SEQ ID NO: 210 | EHEE_rd4_0087.pdb |
| SEQ ID NO: 211 | HHH_rd4_0300.pdb |
| SEQ ID NO: 212 | EEHEE_rd4_0489.pdb |
| SEQ ID NO: 213 | EEHEE_rd3_0160.pdb |
| SEQ ID NO: 214 | HHH_rd4_0304.pdb |
| SEQ ID NO: 215 | HHH_rd4_0887.pdb |
| SEQ ID NO: 216 | EEHEE_rd4_0087.pdb |
| SEQ ID NO: 217 | HHH_rd4_0358.pdb |
| SEQ ID NO: 218 | HHH_rd2_0186.pdb |
| SEQ ID NO: 219 | EEHEE_rd4_0076.pdb |
| SEQ ID NO: 220 | EEHEE_rd4_0399.pdb |
| SEQ ID NO: 221 | EHEE_rd2_1263.pdb |
| SEQ ID NO: 222 | HHH_rd3_0050.pdb |
| SEQ ID NO: 223 | HHH_rd4_0218.pdb |
| SEQ ID NO: 224 | EEHEE_rd4_0063.pdb |
| SEQ ID NO: 225 | HHH_rd4_0005.pdb |
| SEQ ID NO: 226 | HHH_rd2_0089.pdb |
| SEQ ID NO: 227 | HHH_rd2_0104.pdb |
| SEQ ID NO: 228 | EEHEE_rd4_0240.pdb |
| SEQ ID NO: 229 | EHEE_rd4_0162.pdb |
| SEQ ID NO: 230 | HEEH_rd3_0140.pdb |
| SEQ ID NO: 231 | HHH_rd4_0599.pdb |
| SEQ ID NO: 232 | HHH_rd4_0031.pdb |
| SEQ ID NO: 233 | HHH_rd4_0402.pdb |
| SEQ ID NO: 234 | HHH_rd4_0213.pdb |
| SEQ ID NO: 235 | HHH_rd4_0384.pdb |
| SEQ ID NO: 236 | HHH_rd4_0225.pdb |
| SEQ ID NO: 237 | HHH_rd4_0504.pdb |
| SEQ ID NO: 238 | EEHEE_rd4_0182.pdb |
| SEQ ID NO: 239 | EHEE_rd4_0468.pdb |
| SEQ ID NO: 240 | HHH_rd4_0314.pdb |
| SEQ ID NO: 241 | EHEE_rd3_0004.pdb |
| SEQ ID NO: 242 | HHH_rd4_0787.pdb |
| SEQ ID NO: 243 | HHH_rd4_0832.pdb |
| SEQ ID NO: 244 | HHH_rd4_0400.pdb |
| SEQ ID NO: 245 | HHH_rd3_0122.pdb |
| SEQ ID NO: 246 | HHH_rd4_0737.pdb |
| SEQ ID NO: 247 | EHEE_rd4_0195.pdb |
| SEQ ID NO: 248 | EHEE_rd4_0017.pdb |
| SEQ ID NO: 249 | EHEE_rd2_0801.pdb |
| SEQ ID NO: 250 | HHH_rd3_0234.pdb |
| SEQ ID NO: 251 | HHH_rd4_0547.pdb |
| SEQ ID NO: 252 | HHH_rd3_0052.pdb |
| SEQ ID NO: 253 | HHH_rd4_0618.pdb |
| SEQ ID NO: 254 | HHH_rd1_0096.pdb |
| SEQ ID NO: 255 | HHH_rd4_0820.pdb |
| SEQ ID NO: 256 | HHH_rd4_0110.pdb |
| SEQ ID NO: 257 | HHH_rd4_0414.pdb |
| SEQ ID NO: 258 | HHH_rd4_0877.pdb |
| SEQ ID NO: 259 | HHH_rd4_0103.pdb |
| SEQ ID NO: 260 | EEHEE_rd4_0153.pdb |
| SEQ ID NO: 261 | EEHEE_rd4_0028.pdb |
| SEQ ID NO: 262 | HHH_rd4_0333.pdb |
| SEQ ID NO: 263 | HHH_rd4_05551.pdb |
| SEQ ID NO: 264 | HHH_rd2_0084.pdb |
| SEQ ID NO: 265 | HHH_rd4_0227.pdb |
| SEQ ID NO: 266 | HHH_rd4_0811.pdb |
| SEQ ID NO: 267 | HHH_rd4_0462.pdb |
| SEQ ID NO: 268 | HHH_rd4_0428.pdb |
| SEQ ID NO: 269 | HHH_rd4_0348.pdb |
| SEQ ID NO: 270 | HHH_rd4_0827.pdb |
| SEQ ID NO: 271 | EEHEE_rd4_0050.pdb |
| SEQ ID NO: 272 | HHH_rd4_0155.pdb |
| SEQ ID NO: 273 | HHH_rd3_0079.pdb |
| SEQ ID NO: 274 | HHH_rd4_0810.pdb |
| SEQ ID NO: 275 | EHEE_rd4_0012.pdb |
| SEQ ID NO: 276 | HHH_rd4_0386.pdb |
| SEQ ID NO: 277 | EEHEE_rd4_0079.pdb |
| SEQ ID NO: 278 | HHH_rd4_0343.pdb |
| SEQ ID NO: 279 | HHH_rd4_0782.pdb |
| SEQ ID NO: 280 | HHH_rd1_0406.pdb |
| SEQ ID NO: 281 | HHH_rd4_0085.pdb |
| SEQ ID NO: 282 | HHH_rd3_0012.pdb |
| SEQ ID NO: 283 | EEHEE_rd3_1630.pdb |
| SEQ ID NO: 284 | HHH_rd4_0092.pdb |
| SEQ ID NO: 285 | HHH_rd3_0061.pdb |
| SEQ ID NO: 286 | HHH_rd2_0091.pdb |
| SEQ ID NO: 287 | HHH_rd4_0676.pdb |
| SEQ ID NO: 288 | EHEE_rd4_0467.pdb |
| SEQ ID NO: 289 | HHH_rd4_0550.pdb |
| SEQ ID NO: 290 | EEHEE_rd4_0011.pdb |
| SEQ ID NO: 291 | HHH_rd4_0081.pdb |
| SEQ ID NO: 292 | HHH_rd2_0235.pdb |
| SEQ ID NO: 293 | HHH_rd4_0812.pdb |
| SEQ ID NO: 294 | HHH_rd4_0514.pdb |
| SEQ ID NO: 295 | HHH_rd4_0870.pdb |
| SEQ ID NO: 296 | HHH_rd4_0882.pdb |
| SEQ ID NO: 297 | EHEE_rd4_0064.pdb |
| SEQ ID NO: 298 | HHH_rd4_0317.pdb |
| SEQ ID NO: 299 | HHH_rd1_0076.pdb |
| SEQ ID NO: 300 | HHH_rd4_0556.pdb |
| SEQ ID NO: 301 | HHH_rd4_0055.pdb |
| SEQ ID NO: 302 | HHH_rd4_0768.pdb |
| SEQ ID NO: 303 | HHH_rd4_0590.pdb |
| SEQ ID NO: 304 | HHH_rd4_0154.pdb |
| SEQ ID NO: 305 | EHEE_rd4_0713.pdb |
| SEQ ID NO: 306 | HHH_rd4_0606.pdb |
| SEQ ID NO: 307 | HHH_rd4_0609.pdb |
| SEQ ID NO: 308 | HHH_rd4_0332.pdb |
| SEQ ID NO: 309 | EEHEE_rd4_0384.pdb |
| SEQ ID NO: 310 | HHH_rd4_0098.pdb |
| SEQ ID NO: 311 | HHH_rd4_0200.pdb |
| SEQ ID NO: 312 | EHEE_rd3_0233.pdb |
| SEQ ID NO: 313 | HHH_rd4_0868.pdb |
| SEQ ID NO: 314 | HHH_rd4_0244.pdb |
| SEQ ID NO: 315 | HHH_rd4_0087.pdb |
| SEQ ID NO: 316 | HHH_rd4_0670.pdb |
| SEQ ID NO: 317 | HHH_rd2_0088.pdb |
| SEQ ID NO: 318 | EEHEE_rd4_0014.pdb |
| SEQ ID NO: 319 | HHH_rd4_0372.pdb |
| SEQ ID NO: 320 | HHH_rd1_0089.pdb |
| SEQ ID NO: 321 | HHH_rd2_0247.pdb |
| SEQ ID NO: 322 | HHH_rd4_0346.pdb |
| SEQ ID NO: 323 | HHH_rd4_0066.pdb |
| SEQ ID NO: 324 | HHH_rd1_0084.pdb |
| SEQ ID NO: 325 | HHH_rd4_0658.pdb |
| SEQ ID NO: 326 | HHH_rd4_0602.pdb |
| SEQ ID NO: 327 | EHEE_rd4_0183.pdb |
| SEQ ID NO: 328 | HHH_rd4_0760.pdb |
| SEQ ID NO: 329 | EHEE_rd4_0043.pdb |
| SEQ ID NO: 330 | HHH_rd4_0163.pdb |
| SEQ ID NO: 331 | EHEE_rd3_0024.pdb |
| SEQ ID NO: 332 | EEHEE_rd4_0298.pdb |
| SEQ ID NO: 333 | HHH_rd2_0075.pdb |
| SEQ ID NO: 334 | HHH_rd2_0086.pdb |
| SEQ ID NO: 335 | HHH_rd2_0227.pdb |
| SEQ ID NO: 336 | HHH_rd4_0041.pdb |
| SEQ ID NO: 337 | HHH_rd3_0060.pdb |
| SEQ ID NO: 338 | HHH_rd4_0139.pdb |
| SEQ ID NO: 339 | HHH_rd4_0038.pdb |
| SEQ ID NO: 340 | EEHEE_rd4_0418.pdb |
| SEQ ID NO: 341 | EEHEE_rd4_0241.pdb |
| SEQ ID NO: 342 | HHH_rd4_0344.pdb |
| SEQ ID NO: 343 | HHH_rd4_0518.pdb |
| SEQ ID NO: 344 | HHH_rd4_0382.pdb |
| SEQ ID NO: 345 | HHH_rd4_0014.pdb |
| SEQ ID NO: 346 | EHEE_rd4_0826.pdb |
| SEQ ID NO: 347 | HHH_rd1_0112.pdb |
| SEQ ID NO: 348 | HHH_rd4_0643.pdb |
| SEQ ID NO: 349 | HHH_rd3_0094.pdb |
| SEQ ID NO: 350 | EHEE_rd4_0125.pdb |
| SEQ ID NO: 351 | HHH_rd4_0702.pdb |
| SEQ ID NO: 352 | HHH_rd4_0717.pdb |
| SEQ ID NO: 353 | EEHEE_rd4_0064.pdb |
| SEQ ID NO: 354 | HHH_rd4_0629.pdb |
| SEQ ID NO: 355 | HHH_rd4_0302.pdb |
| SEQ ID NO: 356 | HHH_rd4_0011.pdb |
| SEQ ID NO: 357 | HHH_rd4_0094.pdb |
| SEQ ID NO: 358 | HHH_rd4_0086.pdb |
| SEQ ID NO: 359 | HHH_rd3_0160.pdb |
| SEQ ID NO: 360 | HHH_rd4_0409.pdb |
| SEQ ID NO: 361 | HHH_rd2_0097.pdb |
| SEQ ID NO: 362 | HHH_rd4_0746.pdb |
| SEQ ID NO: 363 | HHH_rd1_0083.pdb |
| SEQ ID NO: 364 | HHH_rd4_0034.pdb |
| SEQ ID NO: 365 | EHEE_rd4_0202.pdb |
| SEQ ID NO: 366 | EEHEE_rd4_0229.pdb |
| SEQ ID NO: 367 | HHH_rd4_0903.pdb |
| SEQ ID NO: 368 | HHH_rd4_0355.pdb |
| SEQ ID NO: 369 | HHH_rd4_0390.pdb |
| SEQ ID NO: 370 | HHH_rd3_0239.pdb |
| SEQ ID NO: 371 | HHH_rd4_0705.pdb |
| SEQ ID NO: 372 | EHEE_rd4_0439.pdb |
| SEQ ID NO: 373 | HHH_rd4_0204.pdb |
| SEQ ID NO: 374 | EEHEE_rd4_0570.pdb |
| SEQ ID NO: 375 | EEHEE_rd4_0071.pdb |
| SEQ ID NO: 376 | HHH_rd4_0566.pdb |
| SEQ ID NO: 377 | HHH_rd4_0202.pdb |
| SEQ ID NO: 378 | HHH_rd4_0181.pdb |
| SEQ ID NO: 379 | EEHEE_rd3_1685.pdb |
| SEQ ID NO: 380 | HHH_rd4_0252.pdb |
| SEQ ID NO: 381 | HHH_rd4_0064.pdb |
| SEQ ID NO: 382 | HHH_rd4_0128.pdb |
| SEQ ID NO: 383 | EEHEE_rd4_0350.pdb |
| SEQ ID NO: 384 | EEHEE_rd4_0275.pdb |
| SEQ ID NO: 385 | HHH_rd3_0065.pdb |
| SEQ ID NO: 386 | EEHEE_rd4_0048.pdb |
| SEQ ID NO: 387 | EEHEE_jd4_0006.pdb |
| SEQ ID NO: 388 | HHH_rd4_0733.pdb |
| SEQ ID NO: 389 | HHH_rd3_0051.pdb |
| SEQ ID NO: 390 | EEHEE_rd4_0260.pdb |
| SEQ ID NO: 391 | HHH_rd4_0977.pdb |
| SEQ ID NO: 392 | HHH_rd4_0738.pdb |
| SEQ ID NO: 393 | HHH_rd4_0740.pdb |
| SEQ ID NO: 394 | HHH_rd4_0715.pdb |
| SEQ ID NO: 395 | EEHEE_rd4_0698.pdb |
| SEQ ID NO: 396 | HHH_rd4_0342.pdb |
| SEQ ID NO: 397 | HHH_rd1_0115.pdb |
| SEQ ID NO: 398 | HHH_rd2_0119.pdb |
| SEQ ID NO: 399 | EHEE_rd4_0451.pdb |
| SEQ ID NO: 400 | HHH_rd4_0095.pdb |
| SEQ ID NO: 401 | HHH_rd4_0107.pdb |
| SEQ ID NO: 402 | EHEE_rd4_0101.pdb |
| SEQ ID NO: 403 | EHEE_rd2_1003.pdb |
| SEQ ID NO: 404 | HHH_rd4_0152.pdb |
| SEQ ID NO: 405 | EHEE_rd4_0488.pdb |
| SEQ ID NO: 406 | EEHEE_rd4_0078.pdb |
| SEQ ID NO: 407 | HHH_rd4_0521.pdb |
| SEQ ID NO: 408 | HHH_rd2_0194.pdb |
| SEQ ID NO: 409 | HHH_rd4_0601.pdb |
| SEQ ID NO: 410 | HHH_rd2_0135.pdb |
| SEQ ID NO: 411 | HHH_rd4_0802.pdb |
| SEQ ID NO: 412 | EHEE_rd4_0021.pdb |
| SEQ ID NO: 413 | HHH_rd2_0132.pdb |
| SEQ ID NO: 414 | HHH_rd3_0240.pdb |
| SEQ ID NO: 415 | HHH_rd4_0048.pdb |
| SEQ ID NO: 416 | HHH_rd1_0411.pdb |
| SEQ ID NO: 417 | HHH_rd3_0230.pdb |
| SEQ ID NO: 418 | EEHEE_rd4_0210.pdb |
| SEQ ID NO: 419 | EEHEE_rd3_0654.pdb |
| SEQ ID NO: 420 | EHEE_rd2_0979.pdb |
| SEQ ID NO: 421 | HHH_rd1_0028.pdb |
| SEQ ID NO: 422 | HHH_rd2_0239.pdb |
| SEQ ID NO: 423 | HHH_rd2_0156.pdb |
| SEQ ID NO: 424 | HHH_rd3_0195.pdb |
| SEQ ID NO: 425 | EEHEE_rd4_0001.pdb |
| SEQ ID NO: 426 | EEHEE_rd4_0070.pdb |
| SEQ ID NO: 427 | HHH_rd4_0867.pdb |
| SEQ ID NO: 428 | HHH_rd4_0463.pdb |
| SEQ ID NO: 429 | HHH_rd4_0429.pdb |
| SEQ ID NO: 430 | EEHEE_rd4_0160.pdb |
| SEQ ID NO: 431 | EEHEE_rd4_0208.pdb |
| SEQ ID NO: 432 | HHH_rd4_0053.pdb |
| SEQ ID NO: 433 | EHEE_rd4_0158.pdb |
| SEQ ID NO: 434 | HHH_rd2_0234.pdb |
| SEQ ID NO: 435 | EHEE_rd2_0176.pdb |
| SEQ ID NO: 436 | HHH_rd1_0102.pdb |
| SEQ ID NO: 437 | HHH_rd4_0454.pdb |
| SEQ ID NO: 438 | HHH_rd4_0326.pdb |
| SEQ ID NO: 439 | HHH_rd4_0593.pdb |
| SEQ ID NO: 440 | EHEE_rd3_0015.pdb |
| SEQ ID NO: 441 | HHH_rd4_{——}0656.pdb |
| SEQ ID NO: 442 | HHH_rd1_0533.pdb |
| SEQ ID NO: 443 | EHEE_rd4_0972.pdb |
| SEQ ID NO: 444 | HHH_rd4_0534.pdb |
| SEQ ID NO: 445 | EHEE_rd2_0772.pdb |
| SEQ ID NO: 446 | EEHEE_rd4_0023.pdb |
| SEQ ID NO: 447 | HHH_rd3_0151.pdb |
| SEQ ID NO: 448 | HHH_rd3_0219.pdb |
| SEQ ID NO: 449 | HHH_rd4_0800.pdb |
| SEQ ID NO: 450 | EEHEE_rd4_0221.pdb |
| SEQ ID NO: 451 | HHH_rd4_0338.pdb |
| SEQ ID NO: 452 | HHH_rd4_0079.pdb |
| SEQ ID NO: 453 | HHH_rd4_0837.pdb |
| SEQ ID NO: 454 | HHH_rd4_0866.pdb |
| SEQ ID NO: 455 | HHH_rd1_0307.pdb |
| SEQ ID NO: 456 | HHH_rd3_0177.pdb |
| SEQ ID NO: 457 | EEHEE_rd4_0125.pdb |
| SEQ ID NO: 458 | HHH_rd3_0213.pdb |
| SEQ ID NO: 459 | EEHEE_rd3_1244.pdb |
| SEQ ID NO: 460 | EEHEE_rd4_0870.pdb |
| SEQ ID NO: 461 | HHH_rd1_0120.pdb |
| SEQ ID NO: 462 | EEHEE_rd4_0256.pdb |
| SEQ ID NO: 463 | EEHEE_rd4_0062.pdb |
| SEQ ID NO: 464 | HHH_rd4_0285.pdb |
| SEQ ID NO: 465 | EEHEE_rd4_0024.pdb |
| SEQ ID NO: 466 | EHEE_rd4_0177.pdb |
| SEQ ID NO: 467 | EEHEE_rd4_0327.pdb |
| SEQ ID NO: 468 | HHH_rd2_0155.pdb |
| SEQ ID NO: 469 | HHH_rd4_0310.pdb |
| SEQ ID NO: 470 | HHH_rd1_0260.pdb |
| SEQ ID NO: 471 | HHH_rd4_0461.pdb |
| SEQ ID NO: 472 | EHEE_rd2_0251.pdb |
| SEQ ID NO: 473 | EEHEE_rd3_1034.pdb |
| SEQ ID NO: 474 | EHEE_rd4_0571.pdb |
| SEQ ID NO: 475 | HHH_rd2_0019.pdb |
| SEQ ID NO: 476 | HHH_rd4_0196.pdb |
| SEQ ID NO: 477 | EHEE_rd4_0657.pdb |
| SEQ ID NO: 478 | EHEE_rd4_0630.pdb |
| SEQ ID NO: 479 | HHH_rd4_0567.pdb |
| SEQ ID NO: 480 | EHEE_rd2_0473.pdb |
| SEQ ID NO: 481 | HHH_rd4_0404.pdb |
| SEQ ID NO: 482 | HHH_rd4_0089.pdb |
| SEQ ID NO: 483 | EEHEE_rd4_0664.pdb |
| SEQ ID NO: 484 | HHH_rd4_0794.pdb |
| SEQ ID NO: 485 | EHEE_rd3_0011.pdb |
| SEQ ID NO: 486 | HHH_rd2_0221.pdb |
| SEQ ID NO: 487 | HHH_rd4_0776.pdb |
| SEQ ID NO: 488 | HHH_rd4_0950.pdb |
| SEQ ID NO: 489 | HHH_rd4_0273.pdb |
| SEQ ID NO: 490 | HHH_rd4_0265.pdb |
| SEQ ID NO: 491 | HHH_rd4_0138.pdb |
| SEQ ID NO: 492 | HHH_rd4_0259.pdb |
| SEQ ID NO: 493 | HHH_rd4_0296.pdb |
| SEQ ID NO: 494 | HHH_rd4_0587.pdb |
| SEQ ID NO: 495 | HHH_rd4_0060.pdb |
| SEQ ID NO: 496 | HHH_rd3_0040.pdb |
| SEQ ID NO: 497 | EHEE_rd4_0365.pdb |
| SEQ ID NO: 498 | HHH_rd4_0592.pdb |
| SEQ ID NO: 499 | HHH_rd4_0269.pdb |
| SEQ ID NO: 500 | EEHEE_rd4_0165.pdb |
| SEQ ID NO: 501 | HHH_rd4_0667.pdb |
| SEQ ID NO: 502 | EEHEE_rd3_0977.pdb |
| SEQ ID NO: 503 | EHEE_rd2_0066.pdb |
| SEQ ID NO: 504 | HHH_rd1_0135.pdb |
| SEQ ID NO: 505 | HHH_rd1_0837.pdb |
| SEQ ID NO: 506 | EHEE_rd4_0325.pdb |
| SEQ ID NO: 507 | HHH_rd4_0320.pdb |
| SEQ ID NO: 508 | HHH_rd1_0082.pdb |
| SEQ ID NO: 509 | HHH_rd1_0224.pdb |
| SEQ ID NO: 510 | EHEE_rd4_0136.pdb |
| SEQ ID NO: 511 | HHH_rd4_0915.pdb |
| SEQ ID NO: 512 | HHH_rd4_0943.pdb |
| SEQ ID NO: 513 | EEHEE_rd4_0429.pdb |
| SEQ ID NO: 514 | EHEE_rd4_0338.pdb |
| SEQ ID NO: 515 | HHH_rd4_0161.pdb |
| SEQ ID NO: 516 | HHH_rd4_0315.pdb |
| SEQ ID NO: 517 | EEHEE_rd4_0190.pdb |
| SEQ ID NO: 518 | HHH_rd3_0097.pdb |
| SEQ ID NO: 519 | EHEE_rd2_0074.pdb |
| SEQ ID NO: 520 | HHH_rd4_0007.pdb |
| SEQ ID NO: 521 | EEHEE_rd4_0007.pdb |
| SEQ ID NO: 522 | HHH_rd4_0399.pdb |
| SEQ ID NO: 523 | EHEE_rd4_0025.pdb |
| SEQ ID NO: 524 | HHH_rd3_0147.pdb |
| SEQ ID NO: 525 | EEHEE_rd4_0069.pdb |
| SEQ ID NO: 526 | HHH_rd4_0226.pdb |
| SEQ ID NO: 527 | EEHEE_rd4_0173.pdb |
| SEQ ID NO: 528 | HHH_rd1_0320.pdb |
| SEQ ID NO: 529 | HHH_rd3_0226.pdb |
| SEQ ID NO: 530 | EHEE_rd4_0833.pdb |
| SEQ ID NO: 531 | HHH_rd4_0578.pdb |
| SEQ ID NO: 532 | EEHEE_rd4_0348.pdb |
| SEQ ID NO: 533 | HHH_rd4_0361.pdb |
| SEQ ID NO: 534 | EEHEE_rd4_0456.pdb |
| SEQ ID NO: 535 | HHH_rd4_0071.pdb |
| SEQ ID NO: 536 | HHH_rd3_0208.pdb |
| SEQ ID NO: 537 | HHH_rd4_0075.pdb |
| SEQ ID NO: 538 | HHH_rd4_0248.pdb |
| SEQ ID NO: 539 | HHH_rd4_0197.pdb |
| SEQ ID NO: 540 | HHH_rd4_0474.pdb |
| SEQ ID NO: 541 | HHH_rd3_0193.pdb |
| SEQ ID NO: 542 | HHH_rd4_0621.pdb |
| SEQ ID NO: 543 | HHH_rd3_0011.pdb |
| SEQ ID NO: 544 | HHH_rd3_0144.pdb |
| SEQ ID NO: 545 | HHH_rd4_0437.pdb |
| SEQ ID NO: 546 | HHH_rd3_0146.pdb |
| SEQ ID NO: 547 | EHEE_rd4_0260.pdb |
| SEQ ID NO: 548 | EHEE_rd1_0101.pdb |
| SEQ ID NO: 549 | HHH_rd4_0120.pdb |
| SEQ ID NO: 550 | HHH_rd1_0247.pdb |
| SEQ ID NO: 551 | HHH_rd4_0854.pdb |
| SEQ ID NO: 552 | EEHEE_rd4_0072.pdb |
| SEQ ID NO: 553 | HHH_rd4_0356.pdb |
| SEQ ID NO: 554 | HHH_rd4_0895.pdb |
| SEQ ID NO: 555 | EEHEE_rd4_0197.pdb |
| SEQ ID NO: 556 | EHEE_rd4_0360.pdb |
| SEQ ID NO: 557 | HHH_rd4_0600.pdb |
| SEQ ID NO: 558 | HHH_rd4_0914.pdb |
| SEQ ID NO: 559 | HHH_rd4_0212.pdb |
| SEQ ID NO: 560 | HHH_rd4_0798.pdb |
| SEQ ID NO: 561 | HHH_rd4_0169.pdb |
| SEQ ID NO: 562 | HHH_rd4_0847.pdb |
| SEQ ID NO: 563 | HHH_rd4_0793.pdb |
| SEQ ID NO: 564 | HHH_rd4_0004.pdb |
| SEQ ID NO: 565 | HHH_rd4_0029.pdb |
| SEQ ID NO: 566 | HHH_rd4_0741.pdb |
| SEQ ID NO: 567 | EHEE_rd4_0821.pdb |
| SEQ ID NO: 568 | HHH_rd4_0287.pdb |
| SEQ ID NO: 569 | EHEE_rd4_0088.pdb |
| SEQ ID NO: 570 | HHH_rd4_0061.pdb |
| SEQ ID NO: 571 | HHH_rd3_0059.pdb |
| SEQ ID NO: 572 | EEHEE_rd4_0785.pdb |
| SEQ ID NO: 573 | EEHEE_rd4_0187.pdb |
| SEQ ID NO: 574 | HHH_rd4_0679.pdb |
| SEQ ID NO: 575 | EEHEE_rd4_0271.pdb |
| SEQ ID NO: 576 | EEHEE_rd4_0353.pdb |
| SEQ ID NO: 577 | HHH_rd4_0935.pdb |
| SEQ ID NO: 578 | HHH_rd4_0305.pdb |
| SEQ ID NO: 579 | HHH_rd4_0584.pdb |
| SEQ ID NO: 580 | HHH_rd4_0201.pdb |
| SEQ ID NO: 581 | HHH_rd4_0523.pdb |
| SEQ ID NO: 582 | HHH_rd4_0940.pdb |
| SEQ ID NO: 583 | HHH_rd4_0195.pdb |
| SEQ ID NO: 584 | HHH_rd4_0571.pdb |
| SEQ ID NO: 585 | HHH_rd4_0840.pdb |
| SEQ ID NO: 586 | HHH_rd3_0100.pdb |
| SEQ ID NO: 587 | HHH_rd1_0343.pdb |
| SEQ ID NO: 588 | HHH_rd4_0563.pdb |
| SEQ ID NO: 589 | HHH_rd4_0232.pdb |
| SEQ ID NO: 590 | HHH_rd4_0445.pdb |
| SEQ ID NO: 591 | EHEE_rd4_0243.pdb |
| SEQ ID NO: 592 | HHH_rd4_0176.pdb |
| SEQ ID NO: 593 | HHH_rd4_0397.pdb |
| SEQ ID NO: 594 | EEHEE_rd4_0617.pdb |
| SEQ ID NO: 595 | HHH_rd4_0186.pdb |
| SEQ ID NO: 596 | HHH_rd1_0111.pdb |
| SEQ ID NO: 597 | HHH_rd3_0073.pdb |
| SEQ ID NO: 598 | EEHEE_rd4_0225.pdb |
| SEQ ID NO: 599 | HHH_rd4_0586.pdb |
| SEQ ID NO: 600 | HHH_rd4_0288.pdb |
| SEQ ID NO: 601 | EEHEE_rd4_0514.pdb |
| SEQ ID NO: 602 | EEHEE_rd4_0232.pdb |
| EEG :0 NO: 603 | EHEE_rd2_0096.pdb |
| SEQ ID NO: 604 | HHH_rd4_0349.pdb |
| SEQ ID NO: 605 | HHH_rd4_0036.pdb |
| SEQ ID NO: 606 | HHH_rd1_0220.pdb |
| SEQ ID NO: 607 | HHH_rd4_0009.pdb |
| SEQ ID NO: 608 | EHEE_rd4_0069.pdb |
| SEQ ID NO: 609 | HHH_rd4_0742.pdb |
| SEQ ID NO: 610 | EHEE_rd4_0321.pdb |
| SEQ ID NO: 611 | EEHEE_rd4_0102.pdb |
| SEQ ID NO: 612 | HHH_rd4_0954.pdb |
| SEQ ID NO: 613 | EHEE_rd4_0154.pdb |
| SEQ ID NO: 614 | EEHEE_rd4_0056.pdb |
| SEQ ID NO: 615 | HHH_rd3_0246.pdb |
| SEQ ID NO: 616 | EHEE_rd2_0001.pdb |
| SEQ ID NO: 617 | HHH_rd3_0224.pdb |
| SEQ ID NO: 618 | HHH_rd2_0021.pdb |
| SEQ ID NO: 619 | HHH_rd4_0561.pdb |
| SEQ ID NO: 620 | HHH_rd3_0212.pdb |
| SEQ ID NO: 621 | EEHEE_rd4_0363.pdb |
| SEQ ID NO: 622 | EEHEE_rd4_0152.pcb |
| SEQ ID NO: 623 | EHEE_rd2_0875.pdb |
| SEQ ID NO: 624 | HEEH_rd3_0466.pdb |
| SEQ ID NO: 625 | HHH_rd4_0991.pdb |
| SEQ ID NO: 626 | HHH_rd1_0251.pdb |
| SEQ ID NO: 627 | HHH_rd3_0116.pdb |
| SEQ ID NO: 628 | HHH_rd4_0146.pdb |
| SEQ ID NO: 629 | EHEE_rd4_0646.pdb |
| SEQ ID NO: 630 | HHH_rd3_0034.pdb |
| SEQ ID NO: 631 | EHEE_rd4_0132.pdb |
| SEQ ID NO: 632 | HHH_rd1_0033.pdb |
| SEQ ID NO: 633 | HHH_rd4_0956.pdb |
| SEQ ID NO: 634 | EHEE_rd4_0167.pdb |
| SEQ ID NO: 635 | HHH_rd4_0930.pdb |
| SEQ ID NO: 636 | HHH_rd4_0243.pdb |
| SEQ ID NO: 637 | HHH_rd4_0799.pdb |
| SEQ ID NO: 638 | HHH_rd4_0617.pdb |
| SEQ ID NO: 639 | HHH_rd4_0816.pdb |
| SEQ ID NO: 640 | HHH_rd4_0899.pdb |
| SEQ ID NO: 641 | HHH_rd3_0015.pdb |
| SEQ ID NO: 642 | HHH_rd4_0239.pdb |
| SEQ ID NO: 643 | EEHEE_rd4_0037.pdb |
| SEQ ID NO: 644 | HHH_rd4_0375.pdb |
| SEQ ID NO: 645 | HHH_rd4_0178.pdb |
| SEQ ID NO: 646 | HHH_rd4_0828.pdb |
| SEQ ID NO: 647 | HHH_rd3_0233.pdb |
| SEQ ID NO: 648 | EHEE_rd4_0673.pdb |
| SEQ ID NO: 649 | HHH_rd4_0111.pdb |
| SEQ ID NO: 650 | EEHEE_rd4_0434.pdb |
| SEQ ID NO: 651 | HHH_rd4_0754.pdb |
| SEQ ID NO: 652 | HHH_rd1_0142.pdb |
| SEQ ID NO: 653 | EEHEE_rd4_0198.pdb |
| SEQ ID NO: 654 | HHH_rd4_0238.pdb |
| SEQ ID NO: 655 | HHH_rd4_0046.pdb |
| SEQ ID NO: 656 | EHEE_rd4_0545.pdb |
| SEQ ID NO: 657 | EEHEE_rd4_0365.pdb |
| SEQ ID NO: 658 | EEHEE_rd4_0887.pdb |
| SEQ ID NO: 659 | EEHEE_rd4_0003.pdb |
| SEQ ID NO: 660 | HHH_rd4_0220.pdb |
| SEQ ID NO: 661 | HHH_rd4_0642.pdb |
| SEQ ID NO: 662 | HHH_rd4_0206.pdb |
| SEQ ID NO: 663 | HHH_rd4_0981.pdb |
| SEQ ID NO: 664 | EEHEE_rd4_0018.pdb |
| SEQ ID NO: 665 | HHH_rd4_0714.pdb |
| SEQ ID NO: 666 | HHH_rd4_0424.pdb |
| SEQ ID NO: 667 | HHH_rd4_0918.pdb |
| SEQ ID NO: 668 | HEEH_rd3_0784.pdb |
| SEQ ID NO: 669 | HHH_rd4_0874.pdb |
| SEQ ID NO: 670 | HHH_rd3_0164.pdb |
| SEQ ID NO: 671 | HHH_rd4_0020.pdb |
| SEQ ID NO: 672 | HHH_rd4_0975.pdb |
| SEQ ID NO: 673 | HHH_rd4_0904.pdb |
| SEQ ID NO: 674 | HHH_rd4_0449.pdb |
| SEQ ID NO: 675 | HHH_rd2_0101.pdb |
| SEQ ID NO: 676 | HHH_rd4_0478.pdb |
| SEQ ID NO: 677 | HHH_rd4_0537.pdb |
| SEQ ID NO: 678 | HHH_rd4_0699.pdb |
| SEQ ID NO: 679 | HHH_rd4_0442.pdb |
| SEQ ID NO: 680 | EEHEE_rd4_0031.pdb |
| SEQ ID NO: 681 | HHH_rd4_0420.pdb |
| SEQ ID NO: 682 | EEHEE_rd4_0497.pdb |
| SEQ ID NO: 683 | EEHEE_rd4_0486.pdb |
| SEQ ID NO: 684 | HHH_rd2_0124.pdb |
| SEQ ID NO: 685 | EHEE_rd4_0224.pdb |
| SEQ ID NO: 686 | EEHEE_rd4_0309.pdb |
| SEQ ID NO: 687 | HHH_rd4_0143.pdb |
| SEQ ID NO: 688 | HHH_rd4_0732.pdb |
| SEQ ID NO: 689 | EHEE_rd4_0140.pdb |
| SEQ ID NO: 690 | EEHEE_rd4_0137.pdb |
| SEQ ID NO: 691 | EEHEE_rd4_0285.pdb |
| SEQ ID NO: 692 | HHH_rd2_0018.pdb |
| SEQ ID NO: 693 | EEHEE_rd4_0295.pdb |
| SEQ ID NO: 694 | HHH_rd3_0235d.pdb |
| SEQ ID NO: 695 | HHH_rd1_0606.pdb |
| SEQ ID NO: 696 | EEHEE_rd4_0341.pdb |
| SEQ ID NO: 697 | EEHEE_rd3_0229.pdb |
| SEQ ID NO: 698 | HEEH_rd3_0260.pdb |
| SEQ ID NO: 699 | HHH_rd4_0023.pdb |
| SEQ ID NO: 700 | HHH_rd4_0266.pdb |
| SEQ ID NO: 701 | HHH_rd2_0148.pdb |
| SEQ ID NO: 702 | EHEE_rd1_0284.pdb |
| SEQ ID NO: 703 | EEHEE_rd4_0204.pdb |
| SEQ ID NO: 704 | HHH_rd4_0190.pdb |
| SEQ ID NO: 705 | EHEE_rd4_0290.pdb |
| SEQ ID NO: 706 | EHEE_rd4_0598.pdb |
| SEQ ID NO: 707 | EEHEE_rd4_0998.pdb |
| SEQ ID NO: 708 | EEHEE_rd4_0104.pdb |
| SEQ ID NO: 709 | HHH_rd4_0221.pdb |
| SEQ ID NO: 710 | HHH_rd4_0135.pdb |
| SEQ ID NO: 711 | HHH_rd4_0831.pdb |
| SEQ ID NO: 712 | HHH_rd4_0664.pdb |
| SEQ ID NO: 713 | HHH_rd4_0447.pdb |
| SEQ ID NO: 714 | HHH_rd4_0841.pdb |
| SEQ ID NO: 715 | HHH_rd2_0133.pdb |
| SEQ ID NO: 716 | HHH_rd4_0908.pdb |
| SEQ ID NO: 717 | HHH_rd4_0234.pdb |
| SEQ ID NO: 718 | EHEE_rd4_0677.pdb |
| SEQ ID NO: 719 | EHEE_rd2_0372.pdb |
| SEQ ID NO: 720 | EEHEE_rd4_0098.pdb |
| SEQ ID NO: 721 | EEHEE_rd4_0647.pdb |
| SEQ ID NO: 722 | HHH_rd4_0174.pdb |
| SEQ ID NO: 723 | HHH_rd3_0032.pdb |
| SEQ ID NO: 724 | HHH_rd3_0055.pdb |
| SEQ ID NO: 725 | HEEH_rd3_0103.pdb |
| SEQ ID NO: 726 | HHH_rd4_0476.pdb |
| SEQ ID NO: 727 | HHH_rd4_0422.pdb |
| SEQ ID NO: 728 | EHEE_rd2_0082.pdb |
| SEQ ID NO: 729 | EHEE_rd3_0003.pdb |
| SEQ ID NO: 730 | HHH_rd1_0590.pdb |
| SEQ ID NO: 731 | HHH_rd3_0049.pdb |
| SEQ ID NO: 732 | HHH_rd4_0165.pdb |
| SEQ ID NO: 733 | HHH_rd4_0117.pdb |
| SEQ ID NO: 734 | HHH_rd4_0736.pdb |
| SEQ ID NO: 735 | EHEE_rd3_0016.pdb |
| SEQ ID NO: 736 | EEHE_rd3_0395.pdb |
| SEQ ID NO: 737 | HHH_rd1_0439.pdb |
| SEQ ID NO: 738 | EEHEE_rd4_0189.pdb |
| SEQ ID NO: 739 | EEHEE_rd4_0553.pdb |
| SEQ ID NO: 740 | EEHEE_rd3_1584.pdb |
| SEQ ID NO: 741 | HHH_rd1_0622.pdb |
| SEQ ID NO: 742 | HHH_rd4_0481.pdb |
| SEQ ID NO: 743 | EHEE_rd4_0448.pdb |
| SEQ ID NO: 744 | HHH_rd3_0026.pdb |
| SEQ ID NO: 745 | HHH_rd4_0230.pdb |
| SEQ ID NO: 746 | HHH_rd4_0401.pdb |
| SEQ ID NO: 747 | HHH_rd4_0880.pdb |
| SEQ ID NO: 748 | HHH_rd4_0692.pdb |
| SEQ ID NO: 749 | EEHEE_rd4_0049.pdb |
| SEQ ID NO: 750 | EEHEE_rd4_0490.pdb |
| SEQ ID NO: 751 | EEHEE_rd4_0093.pdb |
| SEQ ID NO: 752 | HHH_rd4_0444.pdb |
| SEQ ID NO: 753 | HHH_rd4_0739.pdb |
| SEQ ID NO: 754 | HHH_rd3_0017.pdb |
| SEQ ID NO: 755 | HHH_rd1_0233.pdb |
| SEQ ID NO: 756 | EHEE_rd4_0356.pdb |
| SEQ ID NO: 757 | EEHEE_rd4_0448.pdb |
| SEQ ID NO: 758 | HHH_rd3_0068.pdb |
| SEQ ID NO: 759 | HHH_rd4_0298.pdb |
| SEQ ID NO: 760 | HHH_rd4_0941.pdb |
| SEQ ID NO: 761 | HHH_rd4_0982.pdb |
| SEQ ID NO: 762 | HHH_rd4_0147.pdb |
| SEQ ID NO: 763 | HHH_rd4_0529.pdb |
| SEQ ID NO: 764 | HHH_rd4_0538.pdb |
| SEQ ID NO: 765 | HHH_rd3_0124.pdb |
| SEQ ID NO: 766 | HHH_rd2_0056.pdb |
| SEQ ID NO: 767 | EEHEE_rd4_0394.pdb |
| SEQ ID NO: 768 | HHH_rd4_0638.pdb |
| SEQ ID NO: 769 | HHH_rd3_0072.pdb |
| SEQ ID NO: 770 | HHH_rd4_0660.pdb |
| SEQ ID NO: 771 | HHH_rd3_0148.pdb |
| SEQ ID NO: 772 | HHH_rd4_0694.pdb |
| SEQ ID NO: 773 | EEHEE_rd4_0857.pdb |
| SEQ ID NO: 774 | HHH_rd3_0035.pdb |
| SEQ ID NO: 775 | EEHEE_rd3_1692.pdb |
| SEQ ID NO: 776 | HHH_rd4_0299.pdb |
| SEQ ID NO: 777 | HHH_rd1_0756.pdb |
| SEQ ID NO: 778 | HHH_rd3_0201.pdb |
| SEQ ID NO: 779 | HHH_rd3_0194.pdb |
| SEQ ID NO: 780 | HHH_rd4_0158.pdb |
| SEQ ID NO: 781 | HHH_rd4_0250.pdb |
| SEQ ID NO: 782 | HHH_rd4_0082.pdb |
| SEQ ID NO: 783 | HHH_rd4_0406.pdb |
| SEQ ID NO: 784 | EHEE_rd4_0860.pdb |
| SEQ ID NO: 785 | HHH_rd2_0141.pdb |
| SEQ ID NO: 786 | HHH_rd4_0233.pdb |
| SEQ ID NO: 787 | EEHEE_rd4_0373.pdb |
| SEQ ID NO: 788 | EEHEE_rd4_0075.pdb |
| SEQ ID NO: 789 | EHEE_rd4_0028.pdb |
| SEQ ID NO: 790 | EHEE_rd4_0007.pdb |
| SEQ ID NO: 791 | EEHEE_rd4_0067.pdb |
| SEQ ID NO: 792 | HHH_rd4_0362.pdb |
| SEQ ID NO: 793 | HHH_rd4_0387.pdb |
| SEQ ID NO: 794 | HHH_rd1_0801.pdb |
| SEQ ID NO: 795 | HHH_rd3_0185.pdb |
| SEQ ID NO: 796 | HHH_rd4_0570.pdb |
| SEQ ID NO: 797 | HHH_rd1_0376.pdb |
| SEQ ID NO: 798 | EEHEE_rd4_0829.pdb |
| SEQ ID NO: 799 | EEHEE_rd3_0765.pdb |
| SEQ ID NO: 800 | HHH_rd4_0980.pdb |
| SEQ ID NO: 801 | HHH_rd4_0485.pdb |
| SEQ ID NO: 802 | HHH_rd4_0479.pdb |
| SEQ ID NO: 803 | HHH_rd1_0197.pdb |
| SEQ ID NO: 804 | HEEH_rd3_1024.pdb |
| SEQ ID NO: 805 | HHH_rd4_0921.pdb |
| SEQ ID NO: 806 | EHEE_rd4_0283.pdb |
| SEQ ID NO: 807 | HHH_rd4_0192.pdb |
| SEQ ID NO: 808 | HHH_rd4_0217.pdb |
| SEQ ID NO: 809 | HHH_rd4_0016.pdb |
| SEQ ID NO: 810 | HHH_rd4_0826.pdb |
| SEQ ID NO: 811 | EHEE_rd4_0285.pdb |
| SEQ ID NO: 812 | HHH_rd4_0488.pdb |
| SEQ ID NO: 813 | HHH_rd1_0286.pdb |
| SEQ ID NO: 814 | HHH_rd3_0198.pdb |
| SEQ ID NO: 815 | HHH_rd4_0367.pdb |
| SEQ ID NO: 816 | HHH_rd4_0769.pdb |
| SEQ ID NO: 817 | EHEE_rd4_0739.pdb |
| SEQ ID NO: 818 | EHEE_rd4_0252.pdb |
| SEQ ID NO: 819 | EHEE_rd2_0309.pdb |
| SEQ ID NO: 820 | EHEE_rd4_0246.pdb |
| SEQ ID NO: 821 | HHH_rd4_0157.pdb |
| SEQ ID NO: 822 | EHEE_rd4_0280.pdb |
| SEQ ID NO: 823 | HHH_rd2_0002.pdb |
| SEQ ID NO: 824 | HHH_rd1_0855.pdb |
| SEQ ID NO: 825 | EHEE_rd4_0042.pdb |
| SEQ ID NO: 826 | EEHEE_rd4_0156.pdb |
| SEQ ID NO: 827 | EHEE_rd4_0044.pdb |
| SEQ ID NO: 828 | HHH_rd4_0524.pdb |
| SEQ ID NO: 829 | EEHEE_rd4_0626.pdb |
| SEQ ID NO: 830 | EHEE_rd4_0003.pdb |
| SEQ ID NO: 831 | EEHEE_rd4_0349.pdb |
| SEQ ID NO: 832 | HHH_rd4_0885.pdb |
| SEQ ID NO: 833 | HHH_rd4_0247.pdb |
| SEQ ID NO: 834 | EEHEE_rd4_0459.pdb |
| SEQ ID NO: 835 | HHH_rd4_0074.pdb |
| SEQ ID NO: 836 | HHH_rd4_0060.pdb |
| SEQ ID NO: 837 | EHEE_rd4_0071.pdb |
| SEQ ID NO: 838 | HHH_rd4_0460.pdb |
| SEQ ID NO: 839 | HHH_rd4_0170.pdb |
| SEQ ID NO: 840 | HHH_rd4_0425.pdb |
| SEQ ID NO: 841 | EEHEE_rd4_0376.pdb |
| SEQ ID NO: 842 | HHH_rd4_0417.pdb |
| SEQ ID NO: 843 | HHH_rd4_0557.pdb |
| SEQ ID NO: 844 | EEHEE_rd4_0042.pdb |
| SEQ ID NO: 845 | HHH_rd4_0494.pdb |
| SEQ ID NO: 846 | EEHEE_rd3_0343.pdb |
| SEQ ID NO: 847 | HHH_rd4_0121.pdb |
| SEQ ID NO: 848 | EEHEE_rd4_0223.pdb |
| SEQ ID NO: 849 | HHH_rd3_0188.pdb |
| SEQ ID NO: 850 | HHH_rd4_0548.pdb |
| SEQ ID NO: 851 | EHEE_rd4_0463.pdb |
| SEQ ID NO: 852 | EEHEE_rd4_0013.pdb |
| SEQ ID NO: 853 | HHH_rd4_0378.pdb |
| SEQ ID NO: 854 | HHH_rd4_0127.pdb |
| SEQ ID NO: 855 | HHH_rd3_0121.pdb |
| SEQ ID NO: 856 | HHH_rd4_0183.pdb |
| SEQ ID NO: 857 | HHH_rd4_0766.pdb |
| SEQ ID NO: 858 | EHEE_rd4_0253.pdb |
| SEQ ID NO: 859 | HHH_rd4_0345.pdb |
| SEQ ID NO: 860 | EEHEE_rd3_0083.pdb |
| SEQ ID NO: 861 | EHEE_rd4_0068.pdb |
| SEQ ID NO: 862 | HHH_rd4_0018.pdb |
| SEQ ID NO: 863 | HHH_rd3_0161.pdb |
| SEQ ID NO: 864 | HHH_rd4_0690.pdb |
| SEQ ID NO: 865 | HHH_rd4_0090.pdb |
| SEQ ID NO: 866 | EEHEE_rd4_0116.pdb |
| SEQ ID NO: 867 | HHH_rd4_0597.pdb |
| SEQ ID NO: 868 | HHH_rd2_0071.pdb |
| SEQ ID NO: 869 | HHH_rd4_0917.pdb |
| SEQ ID NO: 870 | EHEE_rd4_0398.pdb |
| SEQ ID NO: 871 | EHEE_rd4_0651.pdb |
| SEQ ID NO: 872 | EEHEE_rd3_1466.pdb |
| SEQ ID NO: 873 | HHH_rd4_0686.pdb |
| SEQ ID NO: 874 | EHEE_rd3_0224.pdb |
| SEQ ID NO: 875 | HHH_rd1_0630.pdb |
| SEQ ID NO: 876 | HHH_rd3_0142.pdb |
| SEQ ID NO: 877 | EHEE_rd4_0023.pdb |
| SEQ ID NO: 878 | EEHEE_rd4_0126.pdb |
| SEQ ID NO: 879 | HHH_rd2_0163.pdb |
| SEQ ID NO: 880 | EEHEE_rd4_0763.pdb |
| SEQ ID NO: 881 | HHH_rd2_0215.pdb |
| SEQ ID NO: 882 | HHH_rd4_0850.pdb |
| SEQ ID NO: 883 | EHEE_rd4_0036.pdb |
| SEQ ID NO: 884 | EEHEE_rd4_0146.pdb |
| SEQ ID NO: 885 | HHH_rd4_0603.pdb |
| SEQ ID NO: 886 | EHEE_rd4_0361.pdb |
| SEQ ID NO: 887 | HHH_rd4_0610.pdb |
| SEQ ID NO: 888 | HHH_rd4_0574.pdb |
| SEQ ID NO: 889 | HHH_rd4_0707.pdb |
| SEQ ID NO: 890 | EEHEE_rd4_0021.pdb |
| SEQ ID NO: 891 | EEHEE_rd4_0407.pdb |
| SEQ ID NO: 892 | HHH_rd4_0472.pdb |
| SEQ ID NO: 893 | HHH_rd3_0021.pdb |
| SEQ ID NO: 894 | EEHEE_rd4_0899.pdb |
| SEQ ID NO: 895 | EEHEE_rd4_0008.pdb |
| SEQ ID NO: 896 | EHEE_rd1_0882.pdb |
| SEQ ID NO: 897 | HHH_rd4_0756.pdb |
| SEQ ID NO: 898 | EHEE_rd2_0341.pdb |
| SEQ ID NO: 899 | HHH_rd4_0809.pdb |
| SEQ ID NO: 900 | HHH_rd4_0006.pdb |
| SEQ ID NO: 901 | HHH_rd4_0210.pdb |
| SEQ ID NO: 902 | HHH_rd4_0133.pdb |
| SEQ ID NO: 903 | EHEE_rd3_0064.pdb |
| SEQ ID NO: 904 | HHH_rd1_0021.pdb |
| SEQ ID NO: 905 | HHH_rd3_0095.pdb |
| SEQ ID NO: 906 | HHH_rd1_0190.pdb |
| SEQ ID NO: 907 | EHEE_rd4_0045.pdb |
| SEQ ID NO: 908 | EEHEE_rd4_0132.pdb |
| SEQ ID NO: 909 | HHH_rd4_0129.pdb |
| SEQ ID NO: 910 | EHEE_rd3_0195.pdb |
| SEQ ID NO: 911 | HHH_rd1_0787.pdb |
| SEQ ID NO: 912 | HHH_rd4_0264.pdb |
| SEQ ID NO: 913 | HHH_rd1_0289.pdb |
| SEQ ID NO: 914 | HHH_rd4_0153.pdb |
| SEQ ID NO: 915 | EEHEE_rd4_0328.pdb |
| SEQ ID NO: 916 | EEHEE_rd4_0391.pdb |
| SEQ ID NO: 917 | HHH_rd4_0893.pdb |
| SEQ ID NO: 918 | HHH_rd4_0805.pdb |
| SEQ ID NO: 919 | EHEE_rd4_0938.pdb |
| SEQ ID NO: 920 | HHH_rd4_0966.pdb |
| SEQ ID NO: 921 | EHEE_rd4_0586.pdb |
| SEQ ID NO: 922 | HHH_rd2_0214.pdb |
| SEQ ID NO: 923 | EEHEE_rd4_0268.pdb |
| SEQ ID NO: 924 | HHH_rd4_0576.pdb |
| SEQ ID NO: 925 | HHH_rd4_0309.pdb |
| SEQ ID NO: 926 | EHEE_rd4_0320.pdb |
| SEQ ID NO: 927 | HHH_rd4_0835.pdb |
| SEQ ID NO: 928 | HHH_rd4_0166.pdb |
| SEQ ID NO: 929 | HHH_rd2_0007.pdb |
| SEQ ID NO: 930 | HHH_rd4_0848.pdb |
| SEQ ID NO: 931 | EEHEE_rd4_0754.pdb |
| SEQ ID NO: 932 | EHEE_rd4_0333.pdb |
| SEQ ID NO: 933 | EEHEE_rd4_0519.pdb |
| SEQ ID NO: 934 | HHH_rd4_0486.pdb |
| SEQ ID NO: 935 | HHH_rd2_0173.pdb |
| SEQ ID NO: 936 | EEHEE_rd4_0181.pdb |
| SEQ ID NO: 937 | EEHEE_rd4_0294.pdb |
| SEQ ID NO: 938 | EEHEE_rd4_0140.pdb |
| SEQ ID NO: 939 | EHEE_rd4_0628.pdb |
| SEQ ID NO: 940 | EEHEE_rd4_0016.pdb |
| SEQ ID NO: 941 | EHEE_rd4_0349 pdb |
| SEQ ID NO: 942 | HHH_rd4_0857.pdb |
| SEQ ID NO: 943 | HHH_rd4_0671.pdb |
| SEQ ID NO: 944 | HHH_rd4_0626.pdb |
| SEQ ID NO: 945 | HHH_rd2_0203.pdb |
| SEQ ID NO: 946 | EEHEE_rd3_0468.pdb |
| SEQ ID NO: 947 | HHH_rd4_0377.pdb |
| SEQ ID NO: 948 | HHH_rd4_0759.pdb |
| SEQ ID NO: 949 | HHH_rd4_0710.pdb |
| SEQ ID NO: 950 | HHH_rd4_0453.pdb |
| SEQ ID NO: 951 | HHH_rd4_0436.pdb |
| SEQ ID NO: 952 | HHH_rd4_0354.pdb |
| SEQ ID NO: 953 | HHH_rd4_0564.pdb |
| SEQ ID NO: 954 | EHEE_rd4_0149.pdb |
| SEQ ID NO: 955 | EHEE_rd3_0148.pdb |
| SEQ ID NO: 956 | HHH_rd4_0708.pdb |
| SEQ ID NO: 957 | EEHEE_rd4_0020.pdb |
| SEQ ID NO: 958 | EEHEE_rd4_0020.pdb |
| SEQ ID NO: 959 | EEHEE_rd4_0176.pdb |
| SEQ ID NO: 960 | HHH_rd4_0045.pdb |
| SEQ ID NO: 961 | HHH_rd4_0010.pdb |
| SEQ ID NO: 962 | EEHEE_rd4_0066.pdb |
| SEQ ID NO: 963 | EHEE_rd4_0383.pdb |
| SEQ ID NO: 964 | EEHEE_rd4_0329.pdb |
| SEQ ID NO: 965 | HHH_rd2_0134.pdb |
| SEQ ID NO: 966 | EEHEE_rd4_0226.pdb |
| SEQ ID NO: 967 | HHH_rd4_0172.pdb |
| SEQ ID NO: 968 | HHH_rd4_0878.pdb |
| SEQ ID NO: 969 | HHH_rd4_0123.pdb |
| SEQ ID NO: 970 | HHH_rd4_0187.pdb |
| SEQ ID NO: 971 | EHEE_rd4_0518.pdb |
| SEQ ID NO: 972 | HHH_rd4_0271.pdb |
| SEQ ID NO: 973 | EEHEE_rd4_0797.pdb |
| SEQ ID NO: 974 | EHEE_rd4_0731.pdb |
| SEQ ID NO: 975 | HHH_rd4_0947.pdb |
| SEQ ID NO: 976 | EHEE_rd3_0066.pdb |
| SEQ ID NO: 977 | HHH_rd1_0044.pdb |
| SEQ ID NO: 978 | HHH_rd4_0208.pdb |
| SEQ ID NO: 979 | EEHEE_rd3_1265.pdb |
| SEQ ID NO: 980 | EEHEE_rd4_0250.pdb |
| SEQ ID NO: 981 | EHEE_rd4_0294.pdb |
| SEQ ID NO: 982 | HHH_rd4_0696.pdb |
| SEQ ID NO: 983 | EHEE_rd4_0194.pdb |
| SEQ ID NO: 984 | EHEE_rd2_0981.pdb |
| SEQ ID NO: 985 | HHH_rd1_0353.pdb |
| SEQ ID NO: 986 | HHH_rd3_0197.pdb |
| SEQ ID NO: 987 | EEHEE_rd4_0598.pdb |
| SEQ ID NO: 988 | EEHEE_rd4_0563.pdb |
| SEQ ID NO: 989 | EEHEE_rd3_0170.pdb |
| SEQ ID NO: 990 | HHH_rd4_0328.pdb |
| SEQ ID NO: 991 | EHEE_rd4_0968.pdb |
| SEQ ID NO: 992 | EEHEE_rd4_0595.pdb |
| SEQ ID NO: 993 | HHH_rd4_0923.pdb |
| SEQ ID NO: 994 | EHEE_rd4_0090.pdb |
| SEQ ID NO: 995 | HHH_rd2_0080.pdb |
| SEQ ID NO: 996 | EEHEE_rd4_0027.pdb |
| SEQ ID NO: 997 | HEEH_rd3_1273.pdb |
| SEQ ID NO: 998 | EHEE_rd4_0996.pdb |
| SEQ ID NO: 999 | EEHEE_rd4_0640.pdb |
| SEQ ID NO: 1000 | EEHEE_rd4_0077.pdb |
| SEQ ID NO: 1001 | HHH_rd4_0861.pdb |
| SEQ ID NO: 1002 | EEHEE_rd4_0272.pdb |
| SEQ ID NO: 1003 | EEHEE_rd4_0957.pdb |
| SEQ ID NO: 1004 | HHH_rd3_0083.pdb |
| SEQ ID NO: 1005 | EEHEE_rd4_0358.pdb |
| SEQ ID NO: 1006 | EHEE_rd4_0436.pdb |
| SEQ ID NO: 1007 | EEHEE_rd4_0772.pdb |
| SEQ ID NO: 1008 | EHEE_rd4_0539.pdb |
| SEQ ID NO: 1009 | HHH_rd4_0595.pdb |
| SEQ ID NO: 1010 | HHH_rd1_0470.pdb |
| SEQ ID NO: 1011 | HHH_rd4_0582.pdb |
| SEQ ID NO: 1012 | EHEE_rd2_0861.pdb |
| SEQ ID NO: 1013 | HHH_rd4_0164.pdb |
| SEQ ID NO: 1014 | EEHEE_rd4_0475.pdb |
| SEQ ID NO: 1015 | HHH_rd4_0883.pdb |
| SEQ ID NO: 1016 | HHH_rd4_0323.pdb |
| SEQ ID NO: 1017 | HHH_rd4_0637.pdb |
| SEQ ID NO: 1018 | HHH_rd2_0199.pdb |
| SEQ ID NO: 1019 | HHH_rd4_0341.pdb |
| SEQ ID NO: 1020 | HHH_rd3_0176.pdb |
| SEQ ID NO: 1021 | HHH_rd3_0225.pdb |
| SEQ ID NO: 1022 | EHEE_rd4_0111.pdb |
| SEQ ID NO: 1023 | HHH_rd4_0275.pdb |
| SEQ ID NO: 1024 | HHH_rd3_0183.ptfb |
| SEQ ID NO: 1025 | HHH_rd4_0958.pdb |
| SEQ ID NO: 1026 | EHEE_rd4_0573.pdb |
| SEQ ID NO: 1027 | HHH_rd1_0843.pdb |
| SEQ ID NO: 1028 | EHEE_rd3_0093.pdb |
| SEQ ID NO: 1029 | HHH_rd4_0990.pdb |
| SEQ ID NO: 1030 | EHEE_rd2_0235.pdb |
| SEQ ID NO: 1031 | EEHEE_rd4_0107.pdb |
| SEQ ID NO: 1032 | EEHEE_rd3_1372.pdb |
| SEQ ID NO: 1033 | EHEE_rd4_0909.pdb |
| SEQ ID NO: 1034 | EEHEE_rd4_0194.pdb |
| SEQ ID NO: 1035 | HHH_rd4_0525.pdb |
| SEQ ID NO: 1036 | EHEE_rd4_0371.pdb |
| SEQ ID NO: 1037 | EEHEE_rd4_0030.pdb |
| SEQ ID NO: 1038 | HHH_rd4_0939.pdb |
| SEQ ID NO: 1039 | HEEH_rd3_0223.pdb |
| SEQ ID NO: 1040 | HHH_rd4_0901.pdb |
| SEQ ID NO: 1041 | HHH_rd4_0774.pdb |
| SEQ ID NO: 1042 | HHH_rd4_0431.pdb |
| SEQ ID NO: 1043 | HHH_rd2_0222.pdb |
| SEQ ID NO: 1044 | HHH_rd1_0369 pdb |
| SEQ ID NO: 1045 | HHH_rd3_0023.pdb |
| SEQ ID NO: 1046 | HHH_rd3_0206.pdb |
| SEQ ID NO: 1047 | EHEE_rd4_0758.pdb |
| SEQ ID NO: 1048 | HHH_rd4_0648.pdb |
| SEQ ID NO: 1049 | HHH_rd4_0301.pdb |
| SEQ ID NO: 1050 | EEHEE_rd4_0500.pdb |
| SEQ ID NO: 1051 | EHEE_rd4_0340.pdb |
| SEQ ID NO: 1052 | HHH_rd4_0765.pdb |
| SEQ ID NO: 1053 | EEHEE_rd4_0970.pdb |
| SEQ ID NO: 1054 | HHH_rd3_0210.pdb |
| SEQ ID NO: 1055 | EHEE_rd4_0040.pdb |
| SEQ ID NO: 1056 | HHH_rd4_0502.pdb |
| SEQ ID NO: 1057 | HHH_rd4_0873.pdb |
| SEQ ID NO: 1058 | EHEE_rd4_0458.pdb |
| SEQ ID NO: 1059 | EEHEE_rd4_0334.pdb |
| SEQ ID NO: 1060 | HHH_rd2_0145.pdb |
| SEQ ID NO: 1061 | HHH_rd4_0331.pdb |
| SEQ ID NO: 1062 | HHH_rd4_0393.pdb |
| SEQ ID NO: 1063 | HHH_rd4_0324.pdb |
| SEQ ID NO: 1064 | EEHEE_rd4_0106.pdb |
| SEQ ID NO: 1065 | HHH_rd1_0871.pdb |
| SEQ ID NO: 1066 | EEHEE_rd3_1810.pdb |
| SEQ ID NO: 1067 | HHH_rd4_0281.pdb |
| SEQ ID NO: 1068 | EEHEE_rd4_0989.pdb |
| SEQ ID NO: 1069 | HHH_rd4_0630.pdb |
| SEQ ID NO: 1070 | EEHEE_rd4_0012.pdb |
| SEQ ID NO: 1071 | HHH_rd3_0223.pdb |
| SEQ ID NO: 1072 | HHH_rd2_0225.pdb |
| SEQ ID NO: 1073 | HHH_rd4_0465.pdb |
| SEQ ID NO: 1074 | EEHEE_rd4_0281.pdb |
| SEQ ID NO: 1075 | HHH_rd4_0180.pdb |
| SEQ ID NO: 1076 | HHH_rd4_0019.pdb |
| SEQ ID NO: 1077 | HHH_rd4_0813.pdb |
| SEQ ID NO: 1078 | EEHEE_rd4_0862.pdb |
| SEQ ID NO: 1079 | EEHEE_rd3_1716.pdb |
| SEQ ID NO: 1080 | EHEE_rd4_0288.pdb |
| SEQ ID NO: 1081 | HHH_rd4_0987.pdb |
| SEQ ID NO: 1082 | HHH_rd4_0191.pdb |
| SEQ ID NO: 1083 | EEHEE_rd4_0017.pdb |
| SEQ ID NO: 1084 | HHH_rd4_0185.pdb |
| SEQ ID NO: 1085 | HHH_rd4_0685.pdb |
| SEQ ID NO: 1086 | HHH_rd4_0070.pdb |
| SEQ ID NO: 1087 | HHH_rd4_0099.pdb |
| SEQ ID NO: 1088 | EEHEE_rd4_0302.pdb |
| SEQ ID NO: 1089 | HHH_rd2_0206.pdb |
| SEQ ID NO: 1090 | EEHEE_rd4_0184.pdb |
| SEQ ID NO: 1091 | EHEE_rd4_0250.pdb |
| SEQ ID NO: 1092 | EHEE_rd4_0139.pdb |
| SEQ ID NO: 1093 | HHH_rd4_0083.pdb |
| SEQ ID NO: 1094 | HHH_rd4_0131.pdb |
| SEQ ID NO: 1095 | EHEE_rd2_0273.pdb |
| SEQ ID NO: 1096 | EEHEE_rd3_0648.pdb |
| SEQ ID NO: 1097 | EHEE_rd4_0686.pdb |
| SEQ ID NO: 1098 | EEHEE_rd4_0142.pdb |
| SEQ ID NO: 1099 | EEHEE_rd4_0458.pdb |
| SEQ ID NO: 1100 | EEHEE_rd4_0121.pdb |
| SEQ ID NO: 1101 | HHH_rd4_0651.pdb |
| SEQ ID NO: 1102 | HHH_rd2_0153.pdb |
| SEQ ID NO: 1103 | EEHEE_rd4_0356.pdb |
| SEQ ID NO: 1104 | EEHEE_rd4_0115.pdb |
| SEQ ID NO: 1105 | HHH_rd2_0017.pdb |
| SEQ ID NO: 1106 | HHH_rd4_0907.pdb |
| SEQ ID NO: 1107 | EHEE_rd4_0155.pdb |
| SEQ ID NO: 1108 | HHH_rd3_0127.pdb |
| SEQ ID NO: 1109 | EHEE_rd4_0976.pdb |
| SEQ ID NO: 1110 | HHH_rd4_0219.pdb |
| SEQ ID NO: 1111 | HEEH_rd3_0736.pdb |
| SEQ ID NO: 1112 | HHH_rd4_0398.pdb |
| SEQ ID NO: 1113 | HHH_rd4_0339.pdb |
| SEQ ID NO: 1114 | HHH_rd4_0695.pdb |
| SEQ ID NO: 1115 | HHH_rd3_0086.pdb |
| SEQ ID NO: 1116 | EEHEE_rd4_0212.pdb |
| SEQ ID NO: 1117 | EEHEE_rd4_0255.pdb |
| SEQ ID NO: 1118 | EEHEE_rd4_0525.pdb |
| SEQ ID NO: 1119 | HHH_rd4_0108.pdb |
| SEQ ID NO: 1120 | HHH_rd3_0053.pdb |
| SEQ ID NO: 1121 | HHH_rd4_0792.pdb |
| SEQ ID NO: 1122 | EEHEE_rd4_0009.pdb |
| SEQ ID NO: 1123 | HHH_rd4_0394.pdb |
| SEQ ID NO: 1124 | EEHEE_rd4_0476.pdb |
| SEQ ID NO: 1125 | EHEE_rd2_0271.pdb |
| SEQ ID NO: 1126 | HHH_rd4_0267.pdb |
| SEQ ID NO: 1127 | HHH_rd3_0106.pdb |
| SEQ ID NO: 1128 | EEHEE_rd4_0141.pdb |
| SEQ ID NO: 1129 | EHEE_rd4_0187.pdb |
| SEQ ID NO: 1130 | EEHEE_rd3_1367.pdb |
| SEQ ID NO: 1131 | HHH_rd1_0026.pdb |
| SEQ ID NO: 1132 | EHEE_rd3_0029.pdb |
| SEQ ID NO: 1133 | HHH_rd4_0970.pdb |
| SEQ ID NO: 1134 | HHH_rd4_0615 pdb |
| SEQ ID NO: 1135 | HHH_rd4_0585.pdb |
| SEQ ID NO: 1136 | EEHEE_rd3_1079.pdb |
| SEQ ID NO: 1137 | EHEE_rd2_0475.pdb |
| SEQ ID NO: 1138 | HHH_rd4_0489.pdb |
| SEQ ID NO: 1139 | EEHEE_rd4_0065.pdb |
| SEQ ID NO: 1140 | EHEE_rd4_0033.pdb |
| SEQ ID NO: 1141 | HHH_rd4_0065.pdb |
| SEQ ID NO: 1142 | HEEH_rd4_0053.pdb |
| SEQ ID NO: 1143 | EEHEE_rd4.0249.pdb |
| SEQ ID NO: 1144 | EEHEE_rd4_0535.pdb |
| SEQ ID NO: 1145 | HHH_rd4_0091.pdb |
| SEQ ID NO: 1146 | EEHEE_rd4_0154.pdb |
| SEQ ID NO: 1147 | HHH_rd4_0415.pdb |
| SEQ ID NO: 1148 | EEHEE_rd3_1669.pdb |
| SEQ ID NO: 1149 | EHEE_rd4_0097.pdb |
| SEQ ID NO: 1150 | EEHEE_rd4_0417.pdb |
| SEQ ID NO: 1151 | EEHEE_rd4_0439.pdb |
| SEQ ID NO: 1152 | EEHEE_rd3_0988.pdb |
| SEQ ID NO: 1153 | EEHEE_rd4_0149.pdb |
| SEQ ID NO: 1154 | HHH_rd3_0117.pdb |
| SEQ ID NO: 1155 | HHH_rd4_0939.pdb |
| SEQ ID NO: 1156 | EEHEE_rd4_0586.pdb |
| SEQ ID NO: 1157 | EEHEE_rd3_1790.pdb |
| SEQ ID NO: 1158 | HHH_rd4_0771.pdb |
| SEQ ID NO: 1159 | HHH_rd1_0850.pdb |
| SEQ ID NO: 1160 | HHH_rd2_0241.pdb |
| SEQ ID NO: 1161 | EEHEE_rd4_0631.pdb |
| SEQ ID NO: 1162 | HHH_rd4_0024.pdb |
| SEQ ID NO: 1163 | EEHEE_rd4_0891.pdb |
| SEQ ID NO: 1164 | EHEE_rd4_0073.pdb |
| SEQ ID NO: 1165 | HEEH_rd3_1054.pdb |
| SEQ ID NO: 1166 | EHEE_rd2_0190.pdb |
| SEQ ID NO: 1167 | HHH_rd3_0056.pdb |
| SEQ ID NO: 1168 | EEHEE_rd4_0157.pdb |
| SEQ ID NO: 1169 | EEHEE_rd3_1627.pdb |
| SEQ ID NO: 1170 | EEHEE_rd4_0163.pdb |
| SEQ ID NO: 1171 | HHH_rd1_0608.pdb |
| SEQ ID NO: 1172 | EHEE_rd4_0244.pdb |
| SEQ ID NO: 1173 | HHH_rd1_0426.pdb |
| SEQ ID NO: 1174 | EEHEE_rd4_0581.pdb |
| SEQ ID NO: 1175 | EEHEE_rd3_1353.pdb |
| SEQ ID NO: 1176 | EHEE_rd4_0172.pdb |
| SEQ ID NO: 1177 | EHEE_rd4_0372.pdb |
| SEQ ID NO: 1178 | EHEE_rd4_0086.pdb |
| SEQ ID NO: 1179 | HHH_rd4_0653.pdb |
| SEQ ID NO: 1180 | EEHEE_rd4_0010.pdb |
| SEQ ID NO: 1181 | HHH_rd4_0533.pdb |
| SEQ ID NO: 1182 | EEHEE_rd3_1483.pdb |
| SEQ ID NO: 1183 | HHH_rd4_0193.pdb |
| SEQ ID NO: 1184 | HHH_rd4_0999.pdb |
| SEQ ID NO: 1185 | HHH_rd4_0418.pdb |
| SEQ ID NO: 1186 | HHH_rd4_0872.pdb |
| SEQ ID NO: 1187 | EEHEE_rd3_0623.pdb |
| SEQ ID NO: 1188 | HHH_rd4_0931.pdb |
| SEQ ID NO: 1189 | EEHEE_rd4_0639.pdb |
| SEQ ID NO: 1190 | EEHEE_rd4_0451.pdb |
| SEQ ID NO: 1191 | HHH_rd4_0729.pdb |
| SEQ ID NO: 1192 | EEHEE_rd4_0283.pdb |
| SEQ ID NO: 1193 | EEHEE_rd3_0655.pdb |
| SEQ ID NO: 1194 | HHH_rd3_0203.pdb |
| SEQ ID NO: 1195 | HHH_rd4_0242.pdb |
| SEQ ID NO: 1196 | EHEE_rd4_0544.pdb |
| SEQ ID NO: 1197 | HHH_rd3_0062.pdb |
| SEQ ID NO: 1198 | EEHEE_rd4_0055.pdb |
| SEQ ID NO: 1199 | HHH_rd3_0093.pdb |
| SEQ ID NO: 1200 | HHH_rd3_0181.pdb |
| SEQ ID NO: 1201 | HEEH_rd3_0055.pdb |
| SEQ ID NO: 1202 | HHH_rd4_0471.pdb |
| SEQ ID NO: 1203 | HHH_rd1_0258.pdb |
| SEQ ID NO: 1204 | EEHEE_rd4_0084.pdb |
| SEQ ID NO: 1205 | HHH_rd1_0025.pdb |
| SEQ ID NO: 1206 | HHH_rd4_0069.pdb |
| SEQ ID NO: 1207 | HHH_rd4_0862.pdb |
| SEQ ID NO: 1208 | HHH_rd1_0249.pdb |
| SEQ ID NO: 1209 | EHEE_rd4_0392.pdb |
| SEQ ID NO: 1210 | EEHEE_rd4_0123.pdb |
| SEQ ID NO: 1211 | EHEE_rd4_0756.pdb |
| SEQ ID NO: 1212 | EEHEE_rd4_0039.pdb |
| SEQ ID NO: 1213 | HHH_rd4_0655.pdb |
| SEQ ID NO: 1214 | HHH_rd4_0113.pdb |
| SEQ ID NO: 1215 | HHH_rd1_0057.pdb |
| SEQ ID NO: 1216 | HHH_rd4_0263.pdb |
| SEQ ID NO: 1217 | HHH_rd4_0245.pdb |
| SEQ ID NO: 1218 | EHEE_rd4_0605.pdb |
| SEQ ID NO: 1219 | EEHEE_rd4_0537.pdb |
| SEQ ID NO: 1220 | HHH_rd4_0572.pdb |
| SEQ ID NO: 1221 | HHH_rd2_0250.pdb |
| SEQ ID NO: 1222 | EEHEE_rd4_0499.pdb |
| SEQ ID NO: 1223 | HHH_rd3_0129.pdb |
| SEQ ID NO: 1224 | EEHEE_rd4_0886.pdb |
| SEQ ID NO: 1225 | HHH_rd4_0936.pdb |
| SEQ ID NO: 1226 | EEHEE_rd4_0290.pdb |
| SEQ ID NO: 1227 | HHH_rd4_0957.pdb |
| SEQ ID NO: 1228 | HHH_rd4_0763.pdb |
| SEQ ID NO: 1229 | EEHEE_rd4_0025.pdb |
| SEQ ID NO: 1230 | EEHEE_rd4_0521.pdb |
| SEQ ID NO: 1231 | EEHEE_rd4_0663.pdb |
| SEQ ID NO: 1232 | HHH_rd1_0230.pdb |
| SEQ ID NO: 1233 | EEHEE_rd4_0455.pdb |
| SEQ ID NO: 1234 | EEHEE_rd4_0367.pdb |
| SEQ ID NO: 1235 | HHH_rd4_0910.pdb |
| SEQ ID NO: 1236 | EHEE_rd4_0638.pdb |
| SEQ ID NO: 1237 | EEHEE_rd4_0214.pdb |
| SEQ ID NO: 1238 | HHH_rd4_0334.pdb |
| SEQ ID NO: 1239 | HHH_rd4_0969.pdb |
| SEQ ID NO: 1240 | HHH_rd4_0257.pdb |
| SEQ ID NO: 1241 | HHH_rd1_0493.pdb |
| SEQ ID NO: 1242 | HHH_rd4_0209.pdb |
| SEQ ID NO: 1243 | EHEE_rd4_0453.pdb |
| SEQ ID NO: 1244 | EEHEE_rd4_0322.pdb |
| SEQ ID NO: 1245 | HHH_rd4_0916.pdb |
| SEQ ID NO: 1246 | HHH_rd4_0611.pdb |
| SEQ ID NO: 1247 | HHH_rd1_0296.pdb |
| SEQ ID NO: 1248 | EHEE_rd2_1270.pdb |
| SEQ ID NO: 1249 | EHEE_rd4_0322.pdb |
| SEQ ID NO: 1250 | EEHEE_rd4_0621.pdb |
| SEQ ID NO: 1251 | EHEE_rd3_0022.pdb |
| SEQ ID NO: 1252 | EHEE_rd4_0925.pdb |
| SEQ ID NO: 1253 | EEHEE_rd4_0034.pdb |
| SEQ ID NO: 1254 | HHH_rd4_0371.pdb |
| SEQ ID NO: 1255 | HHH_rd4_0072.pdb |
| SEQ ID NO: 1256 | EHEE_rd4_0840.pdb |
| SEQ ID NO: 1257 | EHEE_rd4_0234.pdb |
| SEQ ID NO: 1258 | EEHEE_rd4_0953.pdb |
| SEQ ID NO: 1259 | EEHEE_rd3_0236.pdb |
| SEQ ID NO: 1260 | EEHEE_rd3_1281.pdb |
| SEQ ID NO: 1261 | HHH_rd3_0172.pdb |
| SEQ ID NO: 1262 | HHH_rd4_0519.pdb |
| SEQ ID NO: 1263 | HHH_rd1_0477.pdb |
| SEQ ID NO: 1264 | HHH_rd1_0223.pdb |
| SEQ ID NO: 1265 | EHEE_rd4_0597.pdb |
| SEQ ID NO: 1266 | EEHEE_rd3_0888.pdb |
| SEQ ID NO: 1267 | HHH_rd3_0150.pdb |
| SEQ ID NO: 1268 | EEHEE_rd4_0549.pdb |
| SEQ ID NO: 1269 | EHEE_rd4_0117.pdb |
| SEQ ID NO: 1270 | HHH_rd2_0035.pdb |
| SEQ ID NO: 1271 | HHH_rd4_0329.pdb |
| SEQ ID NO: 1272 | EEHEE_rd4_0236.pdb |
| SEQ ID NO: 1273 | HHH_rd4_0649.pdb |
| SEQ ID NO: 1274 | HHH_rd1_0652.pdb |
| SEQ ID NO: 1275 | HHH_rd4_0583.pdb |
| SEQ ID NO: 1276 | HHH_rd4_0076.pdb |
| SEQ ID NO: 1277 | HHH_rd1_0484.pdb |
| SEQ ID NO: 1278 | HHH_rd4_0286.pdb |
| SEQ ID NO: 1279 | HHH_rd2_0224.pdb |
| SEQ ID NO: 1280 | HHH_rd2_0172.pdb |
| SEQ ID NO: 1281 | HHH_rd4_0948.pdb |
| SEQ ID NO: 1282 | HHH_rd4_0665.pdb |
| SEQ ID NO: 1283 | HEEH_rd3_0212.pdb |
| SEQ ID NO: 1284 | EHEE_rd4_0065.pdb |
| SEQ ID NO: 1285 | HHH_rd3_0128.pdb |
| SEQ ID NO: 1286 | HHH_rd4_0962.pdb |
| SEQ ID NO: 1287 | HHH_rd3_0186.pdb |
| SEQ ID NO: 1288 | EHEE_rd4_0413.pdb |
| SEQ ID NO: 1289 | EEHEE_rd4_0082.pdb |
| SEQ ID NO: 1290 | EEHEE_rd4_0312.pdb |
| SEQ ID NO: 1291 | EHEE_rd4_0048.pdb |
| SEQ ID NO: 1292 | EEHEE_rd4_0080.pdb |
| SEQ ID NO: 1293 | EEHEE_rd3_1234.pdb |
| SEQ ID NO: 1294 | HHH_rd1_0741.pdb |
| SEQ ID NO: 1295 | EHEE_rd4_0999.pdb |
| SEQ ID NO: 1296 | EEHEE_rd4_0266.pdb |
| SEQ ID NO: 1297 | HHH_rd4_0544.pdb |
| SEQ ID NO: 1298 | EHEE_rd4_0567.pdb |
| SEQ ID NO: 1299 | HHH_rd4_0040.pdb |
| SEQ ID NO: 1300 | EEHEE_rd4_0061.pdb |
| SEQ ID NO: 1301 | EHEE_rd4_0104.pdb |
| SEQ ID NO: 1302 | EHEE_rd2_0191.pdb |
| SEQ ID NO: 1303 | HEEH_rd4_0663.pdb |
| SEQ ID NO: 1304 | EHEE_rd4_0066.pdb |
| SEQ ID NO: 1305 | EHEE_rd4_0157.pdb |
| SEQ ID NO: 1306 | EEHEE_rd4_0711.pdb |
| SEQ ID NO: 1307 | EEHEE_rd3_0602.pdb |
| SEQ ID NO: 1308 | HHH_rd1_0432.pdb |
| SEQ ID NO: 1309 | EEHEE_rd4_0751.pdb |
| SEQ ID NO: 1310 | EEHEE_rd4_0511.pdb |
| SEQ ID NO: 1311 | HHH_rd4_0785.pdb |
| SEQ ID NO: 1312 | EEHEE_rd3_0157.pdb |
| SEQ ID NO: 1313 | HHH_rd4_0446.pdb |
| SEQ ID NO: 1314 | EEHEE_rd3_0081.pdb |
| SEQ ID NO: 1315 | EEHEE_rd3_1484.pdb |
| SEQ ID NO: 1316 | HHH_rd2_0081.pdb |
| SEQ ID NO: 1317 | HHH_rd1_0335.pdb |
| SEQ ID NO: 1318 | HHH_rd4_0830.pdb |
| SEQ ID NO: 1319 | EHEE_rd1_0407.pdb |
| SEQ ID NO: 1320 | EEHEE_rd4_0032.pdb |
| SEQ ID NO: 1321 | EEHEE_rd4_0206.pdb |
| SEQ ID NO: 1322 | HHH_rd4_0512.pdb |
| SEQ ID NO: 1323 | HHH_rd4_0403.pdb |
| SEQ ID NO: 1324 | EHEE_rd4_0170.pdb |
| SEQ ID NO: 1325 | HHH_rd4_0558.pdb |
| SEQ ID NO: 1326 | HHH_rd4_0644.pdb |
| SEQ ID NO: 1327 | HHH_rd4_0108.pdb |
| SEQ ID NO: 1328 | EEHEE_rd4_0468.pdb |
| SEQ ID NO: 1329 | EHEE_rd2_1186.pdb |
| SEQ ID NO: 1330 | HHH_rd3_0242.pdb |
| SEQ ID NO: 1331 | EEHEE_rd4_0099.pdb |
| SEQ ID NO: 1332 | HHH_rd3_0111.pdb |
| SEQ ID NO: 1333 | HHH_rd3_0244.pdb |
| SEQ ID NO: 1334 | HHH_rd4_0495.pdb |
| SEQ ID NO: 1335 | HHH_rd1_0609.pdb |
| SEQ ID NO: 1336 | HHH_rd3_0218.pdb |
| SEQ ID NO: 1337 | EEHEE_rd4_0827.pdb |
| SEQ ID NO: 1338 | HHH_rd4_0719.pdb |
| SEQ ID NO: 1339 | HHH_rd4_0322.pdb |
| SEQ ID NO: 1340 | EEHEE_rd4_0203.pdb |
| SEQ ID NO: 1341 | EHEE_rd4_0058.pdb |
| SEQ ID NO: 1342 | HHH_rd1_0574.pdb |
| SEQ ID NO: 1343 | HHH_rd4_0946.pdb |
| SEQ ID NO: 1344 | HHH_rd4_0646.pdb |
| SEQ ID NO: 1345 | HHH_rd4_0370.pdb |
| SEQ ID NO: 1346 | EEHEE_rd3_0256.pdb |
| SEQ ID NO: 1347 | EEHEE_rd4_0251.pdb |
| SEQ ID NO: 1348 | EEHEE_rd4_0917.pdb |
| SEQ ID NO: 1349 | HHH_rd4_0818.pdb |
| SEQ ID NO: 1350 | EHEE_rd4_0962.pdb |
| SEQ ID NO: 1351 | EHEE_rd4_0415.pdb |
| SEQ ID NO: 1352 | EHEE_rd4_0060.pdb |
| SEQ ID NO: 1353 | HHH_rd4_0972.pdb |
| SEQ ID NO: 1354 | HHH_rd2_0151.pdb |
| SEQ ID NO: 1355 | EEHEE_rd4_0700.pdb |
| SEQ ID NO: 1356 | EEHEE_rd4_0649.pdb |
| SEQ ID NO: 1357 | EHEE_rd4_0098.pdb |
| SEQ ID NO: 1358 | EHEE_rd2_0111.pdb |
| SEQ ID NO: 1359 | EEHEE_rd4_0374.pdb |
| SEQ ID NO: 1360 | EEHEE_rd4_0178.pdb |
| SEQ ID NO: 1361 | HHH_rd1_0039.pdb |
| SEQ ID NO: 1362 | HHH_rd4_0368.pdb |
| SEQ ID NO: 1363 | HHH_rd2_0037.pdb |
| SEQ ID NO: 1364 | HHH_rd4_0780.pdb |
| SEQ ID NO: 1365 | EHEE_rd4_0200.pdb |
| SEQ ID NO: 1366 | EHEE_rd4_0874.pdb |
| SEQ ID NO: 1367 | HHH_rd2_0169.pdb |
| SEQ ID NO: 1368 | EEHEE_rd3_0220.pdb |
| SEQ ID NO: 1369 | HHH_rd4_0427.pdb |
| SEQ ID NO: 1370 | HHH_rd4_0669.pdb |
| SEQ ID NO: 1371 | EHEE_rd4_0923.pdb |
| SEQ ID NO: 1372 | EHEE_rd2_0018.pdb |
| SEQ ID NO: 1373 | EHEE_rd4_0947.pdb |
| SEQ ID NO: 1374 | HHH_rd3_0041.pdb |
| SEQ ID NO: 1375 | HHH_rd3_0228.pdb |
| SEQ ID NO: 1376 | EEHEE_rd4_0875.pdb |
| SEQ ID NO: 1377 | HEEH_rd4_0388.pdb |
| SEQ ID NO: 1378 | EEHEE_rd3_1514.pdb |
| SEQ ID NO: 1379 | HHH_rd4_0693.pdb |
| SEQ ID NO: 1380 | EEHEE_rd4_0485.pdb |
| SEQ ID NO: 1381 | HHH_rd4_0124.pdb |
| SEQ ID NO: 1382 | EEHEE_rd4_0392.pdb |
| SEQ ID NO: 1383 | EEHEE_rd4_0702.pdb |
| SEQ ID NO: 1384 | EHEE_rd4_0094.pdb |
| SEQ ID NO: 1385 | HHH_rd1_0769.pdb |
| SEQ ID NO: 1386 | EEHEE_rd4_0410.pdb |
| SEQ ID NO: 1387 | EHEE_rd4_0609.pdb |
| SEQ ID NO: 1388 | HHH_rd4_0388.pdb |
| SEQ ID NO: 1389 | EHEE_rd4_0297.pdb |
| SEQ ID NO: 1390 | EEHEE_rd4_0347.pdb |
| SEQ ID NO: 1391 | HHH_rd2_0050.pdb |
| SEQ ID NO: 1392 | HHH_rd2_0116.pdb |
| SEQ ID NO: 1393 | EEHEE_rd3_0020.pdb |
| SEQ ID NO: 1394 | HHH_rd2_0095.pdb |
| SEQ ID NO: 1395 | HHH_rd4_0411.pdb |
| SEQ ID NO: 1396 | EHEE_rd4_0006.pdb |
| SEQ ID NO: 1397 | EEHEE_rd4_0183.pdb |
| SEQ ID NO: 1398 | EEHEE_rd4_0650.pdb |
| SEQ ID NO: 1399 | HHH_rd4_0268.pdb |
| SEQ ID NO: 1400 | HHH_rd4_0274.pdb |
| SEQ ID NO: 1401 | HHH_rd4_0278.pdb |
| SEQ ID NO: 1402 | EHEE_rd2_0139.pdb |
| SEQ ID NO: 1403 | HHH_rd4_0306.pdb |
| SEQ ID NO: 1404 | EHEE_rd2_0647.pdb |
| SEQ ID NO: 1405 | HHH_rd4_0797.pdb |
| SEQ ID NO: 1406 | EEHEE_rd3_0104.pdb |
| SEQ ID NO: 1407 | EEHEE_rd4_0133.pdb |
| SEQ ID NO: 1408 | HHH_rd4_0929.pdb |
| SEQ ID NO: 1409 | HHH_rd4_0160.pdb |
| SEQ ID NO: 1410 | HHH_rd1_0092.pdb |
| SEQ ID NO: 1411 | EEHEE_rd4_0691.pdb |
| SEQ ID NO: 1412 | HHH_rd1_0125.pdb |
| SEQ ID NO: 1413 | EEHEE_rd3_1818.pdb |
| SEQ ID NO: 1414 | HHH_rd4_0240.pdb |
| SEQ ID NO: 1415 | EEHEE_rd3_0309.pdb |
| SEQ ID NO: 1416 | HHH_rd4_0846.pdb |
| SEQ ID NO: 1417 | EEHEE_rd4_0607.pdb |
| SEQ ID NO: 1418 | EEHEE_rd3_0523.pdb |
| SEQ ID NO: 1419 | EEHEE_rd4_0359.pdb |
| SEQ ID NO: 1420 | EEHEE_rd3_0393.pdb |
| SEQ ID NO: 1421 | EHEE_rd4_0639.pdb |
| SEQ ID NO: 1422 | EHEE_rd1_0338.pdb |
| SEQ ID NO: 1423 | EHEE_rd2_0231.pdb |
| SEQ ID NO: 1424 | EEHEE_rd4_0185.pdb |
| SEQ ID NO: 1425 | HHH_rd1_0580.pdb |
| SEQ ID NO: 1426 | HHH_rdl_0322.pdb |
| SEQ ID NO: 1427 | HHH_rd4_0466.pdb |
| SEQ ID NO: 1428 | HEEH_rd4_0094.pdb |
| SEQ ID NO: 1429 | EHEE_rd4_0145.pdb |
| SEQ ID NO: 1430 | HHH_rd4_0691.pdb |
| SEQ ID NO: 1431 | HHH_rd4_0777.pdb |
| SEQ ID NO: 1432 | HHH_rd4_0712.pdb |
| SEQ ID NO: 1433 | EEHEE_rd3_1706.pdb |
| SEQ ID NO: 1434 | HHH_rd1_0530.pdb |
| SEQ ID NO: 1435 | HHH_rd1_0434.pdb |
| SEQ ID NO: 1436 | HHH_rd4_0605.pdb |
| SEQ ID NO: 1437 | EHEE_rd2_0531.pdb |
| SEQ ID NO: 1438 | EHEE_rd4_0614.pdb |
| SEQ ID NO: 1439 | EEHEE_rd4_0550.pdb |
| SEQ ID NO: 1440 | EHEE_rd4_0592.pdb |
| SEQ ID NO: 1441 | HHH_rd3_0247.pdb |
| SEQ ID NO: 1442 | EEHEE_rd4_0196.pdb |
| SEQ ID NO: 1443 | EEHEE_rd3_1612.pdb |
| SEQ ID NO: 1444 | EHEE_rd4_0530.pdb |
| SEQ ID NO: 1445 | HHH_rd4_0215.pdb |
| SEQ ID NO: 1446 | HHH_rd1_0195.pdb |
| SEQ ID NO: 1447 | EHEE_rd4_0524.pdb |
| SEQ ID NO: 1448 | EHEE_rd4_0509.pdb |
| SEQ ID NO: 1449 | HHH_rd4_0654.pdb |
| SEQ ID NO: 1450 | HHH_rd3_0006.pdb |
| SEQ ID NO: 1451 | EHEE_rd4_1000.pdb |
| SEQ ID NO: 1452 | HHH_rd4_0613.pdb |
| SEQ ID NO: 1453 | EEHEE_rd4_0397.pdb |
| SEQ ID NO: 1454 | HHH_rd4_0535.pdb |
| SEQ ID NO: 1455 | EEHEE_rd4_0463.pdb |
| SEQ ID NO: 1456 | EEHEE_rd4_0460.pdb |
| SEQ ID NO: 1457 | HEEH_rd4_0049.pdb |
| SEQ ID NO: 1458 | HHH_rd4_0553.pdb |
| SEQ ID NO: 1459 | EHEE_rd4_0241.pdb |
| SEQ ID NO: 1460 | EEHEE_rd3_0250.pdb |
| SEQ ID NO: 1461 | HHH_rd4_0173.pdb |
| SEQ ID NO: 1462 | EEHEE_rd4_0284.pdb |
| SEQ ID NO: 1463 | EEHEE_rd4_0520.pdb |
| SEQ ID NO: 1464 | HHH_rd4_0051.pdb |
| SEQ ID NO: 1465 | EHEE_rd4_0743.pdb |
| SEQ ID NO: 1466 | HHH_rd3_0105.pdb |
| SEQ ID NO: 1467 | HHH_rd3_0044.pdb |
| SEQ ID NO: 1468 | EHEE_rd4_0961.pdb |
| SEQ ID NO: 1469 | HHH_rd4_0817.pdb |
| SEQ ID NO: 1470 | EEHEE_rd4_0923.pdb |
| SEQ ID NO: 1471 | HHH_rd3_0159.pdb |
| SEQ ID NO: 1472 | EEHEE_rd4_0543.pdb |
| SEQ ID NO: 1473 | HHH_rd3_0123.pdb |
| SEQ ID NO: 1474 | EHEE_rd4_0701.pdb |
| SEQ ID NO: 1475 | EEHEE_rd4_0242.pdb |
| SEQ ID NO: 1476 | EEHEE_rd3_1503.pdb |
| SEQ ID NO: 1477 | EEHEE_rd4_0019.pdb |
| SEQ ID NO: 1478 | EEHEE_rd4_0233.pdb |
| SEQ ID NO: 1479 | EEHEE_rd4_0273.pdb |
| SEQ ID NO: 1480 | EEHEE_rd3_0055.pdb |
| SEQ ID NO: 1481 | EEHEE_rd4_0569.pdb |
| SEQ ID NO: 1482 | EHEE_rd4_0263.pdb |
| SEQ ID NO: 1483 | HHH_rd4_0405.pdb |
| SEQ ID NO: 1484 | EEHEE_rd4_0258.pdb |
| SEQ ID NO: 1485 | EEHEE_rd_0259.pdb |
| SEQ ID NO: 1486 | HHH_rd4_0490.pdb |
| SEQ ID NO: 1487 | EHEE_rd4_0147.pdb |
| SEQ ID NO: 1488 | EEHEE_rd3_1148.pdb |
| SEQ ID NO: 1489 | EEHEE_rd4_0682.pdb |
| SEQ ID NO: 1490 | EEHEE_rd3_0277.pdb |
| SEQ ID NO: 1491 | HHH_rd4_0786.pdb |
| SEQ ID NO: 1492 | EEHEE_rd3_0028.pdb |
| SEQ ID NO: 1493 | HHH_rd2_0240.pdb |
| SEQ ID NO: 1494 | EHEE_rd4_0223.pdb |
| SEQ ID NO: 1495 | EHEE_rd4_0151.pdb |
| SEQ ID NO: 1496 | HHH_rd2_0149.pdb |
| SEQ ID NO: 1497 | EEHEE_rd4_0842.pdb |
| SEQ ID NO: 1498 | EHEE_rd4_0353.pdb |
| SEQ ID NO: 1499 | HHH_rd4_0730.pdb |
| SEQ ID NO: 1500 | HHH_rd4_0713.pdb |
| SEQ ID NO: 1501 | EHEE_rd3_0087.pdb |
| SEQ ID NO: 1502 | HHH_rd2_0237.pdb |
| SEQ ID NO: 1503 | EEHEE_rd3_0710.pdb |
| SEQ ID NO: 1504 | EHEE_rd3_0040.pdb |
| SEQ ID NO: 1505 | EHEE_rd2_0993.pdb |
| SEQ ID NO: 1506 | HHH_rd4_02300.pdb |
| SEQ ID NO: 1507 | HHH_rd3_0058.pdb |
| SEQ ID NO: 1508 | EHEE_rd2_0207.pdb |
| SEQ ID NO: 1509 | EEHEE_rd4_0119.pdb |
| SEQ ID NO: 1510 | EEHEE_rd4_0308.pdb |
| SEQ ID NO: 1511 | HHH_rd3_0003.pdb |
| SEQ ID NO: 1512 | HHH_rd3_0001.pdb |
| SEQ ID NO: 1513 | EHEE_rd2_1138.pdb |
| SEQ ID NO: 1514 | EEHEE_rd4_0092.pdb |
| SEQ ID NO: 1515 | EHEE_rd4_0877.pdb |
| SEQ ID NO: 1516 | EHEE_rd4_0882.pdb |
| SEQ ID NO: 1517 | EHEE_rd2_0193.pdb |
| SEQ ID NO: 1518 | EEHEE_rd4_0054.pdb |
| SEQ ID NO: 1519 | HHH_rd1_0762.pdb |
| SEQ ID NO: 1520 | EEHEE_rd4_0043.pdb |
| SEQ ID NO: 1521 | HHH_rd4_0224.pdb |
| SEQ ID NO: 1522 | EHEE_rd4_0027.pdb |
| SEQ ID NO: 1523 | HHH_rd4_0852.pdb |
| SEQ ID NO: 1524 | EHEE_rd3_0037.pdb |
| SEQ ID NO: 1525 | EEHEE_rd4_0287.pdb |
| SEQ ID NO: 1526 | HHH_rd3_0140.pdb |
| SEQ ID NO: 1527 | HEEH_rd2_0727.pdb |
| SEQ ID NO: 1528 | EEHEE_rd3_1065.pdb |
| SEQ ID NO: 1529 | EHEE_rd4_0307.pdb |
| SEQ ID NO: 1530 | EEHEE_rd4_0333.pdb |
| SEQ ID NO: 1531 | HHH_rd1_0578.pdb |
| SEQ ID NO: 1532 | EEHEE_rd2_0770.pdb |
| SEQ ID NO: 1533 | EEHEE_rd4_0297.pdb |
| SEQ ID NO: 1534 | EHEE_rd4_0613.pdb |
| SEQ ID NO: 1535 | EEHEE_rd4_0219.pdb |
| SEQ ID NO: 1536 | EEHEE_rd4_0205.pdb |
| SEQ ID NO: 1537 | HHH_rd1_0444.pdb |
| SEQ ID NO: 1538 | HHH_rd1_0472.pdb |
| SEQ ID NO: 1539 | HHH_rd2_0047.pdb |
| SEQ ID NO: 1540 | EEHEE_rd3_1176.pdb |
| SEQ ID NO: 1541 | EEHEE_rd4_0841.pdb |
| SEQ ID NO: 1542 | EHEE_rd4_0761.pdb |
| SEQ ID NO: 1543 | EEHEE_rd4_0784.pdb |
| SEQ ID NO: 1544 | EEHEE_rd3_1558.pdb |
| SEQ ID NO: 1545 | HHH_rd1_0239.pdb |
| SEQ ID NO: 1546 | EEHEE_rd3_0144.pdb |
| SEQ ID NO: 1547 | EHEE_rd3_0014.pdb |
| SEQ ID NO: 1548 | HHH_rd4_0477.pdb |
| SEQ ID NO: 1549 | HHH_rd4_0443.pdb |
| SEQ ID NO: 1550 | EEHEE_rd4_0558.pdb |
| SEQ ID NO: 1551 | HHH_rd3_0030.pdb |
| SEQ ID NO: 1552 | EHEE_rd2_0028.pdb |
| SEQ ID NO: 1553 | HHH_rd4_0360.pdb |
| SEQ ID NO: 1554 | HHH_rd3_0113.pdb |
| SEQ ID NO: 1555 | EEHEE_rd4_0274.pdb |
| SEQ ID NO: 1556 | HHH_rd1_0451.pdb |
| SEQ ID NO: 1557 | HHH_rd3_0166.pdb |
| SEQ ID NO: 1558 | EEHEE_rd4_0471.pdb |
| SEQ ID NO: 1559 | HHH_rd4_0423.pdb |
| SEQ ID NO: 1560 | HHH_rd4_0892.pdb |
| SEQ ID NO: 1561 | EEHEE_rd4_0036.pdb |
| SEQ ID NO: 1562 | HHH_rd4_0450.pdb |
| SEQ ID NO: 1563 | HHH_rd4_0890.pdb |
| SEQ ID NO: 1564 | HHH_rd4_0500.pdb |
| SEQ ID NO: 1565 | HHH_rd4_0672.pdb |
| SEQ ID NO: 1566 | HHH_rd4_0801.pdb |
| SEQ ID NO: 1567 | EHEE_rd4_0295.pdb |
| SEQ ID NO: 1568 | EEHEE_rd4_0731.pdb |
| SEQ ID NO: 1569 | EHEE_rd4_0039.pdb |
| SEQ ID NO: 1570 | EEHEE_rd4_0362.pdb |
| SEQ ID NO: 1571 | EHEE_rd4_0506.pdb |
| SEQ ID NO: 1572 | EEHEE_rd4_0762.pdb |
| SEQ ID NO: 1573 | EHEE_rd2_0195.pdb |
| SEQ ID NO: 1574 | EHEE_rd4_0960.pdb |
| SEQ ID NO: 1575 | HHH_rd4_0568.pdb |
| SEQ ID NO: 1576 | EHEE_rd3_0104.pdb |
| SEQ ID NO: 1577 | EHEE_rd4_0714.pdb |
| SEQ ID NO: 1578 | EEHEE_rd3_0203.pdb |
| SEQ ID NO: 1579 | HHH_rd4_0241.pdb |
| SEQ ID NO: 1580 | EHEE_rd2_0333.pdb |
| SEQ ID NO: 1581 | EEHEE_rd4_0890.pdb |
| SEQ ID NO: 1582 | EEHEE_rd4_0278.pdb |
| SEQ ID NO: 1583 | HHH_rd1_0022.pdb |
| SEQ ID NO: 1584 | HHH_rd4_0364.pdb |
| SEQ ID NO: 1585 | HHH_rd4_0698.pdb |
| SEQ ID NO: 1586 | HHH_rd4_0506.pdb |
| SEQ ID NO: 1587 | EEHEE_rd3_0057.pdb |
| SEQ ID NO: 1588 | EHEE_rd4_0618.pdb |
| SEQ ID NO: 1589 | HHH_rd4_0625.pdb |
| SEQ ID NO: 1590 | HHH_rd3_0108.pdb |
| SEQ ID NO: 1591 | HHH_rd4_0102.pdb |
| SEQ ID NO: 1592 | HHH_rd4_0855.pdb |
| SEQ ID NO: 1593 | HEEH_rd3_0303.pdb |
| SEQ ID NO: 1594 | EEHEE_rd4_0507.pdb |
| SEQ ID NO: 1595 | HHH_rd2_0208.pdb |
| SEQ ID NO: 1596 | EEHEE_rd3_1250.pdb |
| SEQ ID NO: 1597 | EEHEE_rd4_0135.pdb |
| SEQ ID NO: 1598 | HHH_rd4_0661.pdb |
| SEQ ID NO: 1599 | HHH_rd4_0308.pdb |
| SEQ ID NO: 1600 | HHH_rd4_0365.pdb |
| SEQ ID NO: 1601 | EHEE_rd4_0878.pdb |
| SEQ ID NO: 1602 | EEHEE_rd4_0038.pdb |
| SEQ ID NO: 1603 | EHEE_rd4_0444.pdb |
| SEQ ID NO: 1604 | HHH_rd1_0097.pdb |
| SEQ ID NO: 1605 | EEHEE_rd4_0109.pdb |
| SEQ ID NO: 1606 | HEEH_rd3_0731.pdb |
| SEQ ID NO: 1607 | EHEE_rd4_0560.pdb |
| SEQ ID NO: 1608 | HHH_rd4_0389.pdb |
| SEQ ID NO: 1609 | HHH_rd4_0482.pdb |
| SEQ ID NO: 1610 | HHH_rd2_0102.pdb |
| SEQ ID NO: 1611 | EEHEE_rd3_1164.pdb |
| SEQ ID NO: 1612 | EHEE_rd4_0391.pdb |
| SEQ ID NO: 1613 | HHH_rd4_0938.pdb |
| SEQ ID NO: 1614 | EHEE_rd4_0535.pdb |
| SEQ ID NO: 1615 | EEHEE_rd4_0237.pdb |
| SEQ ID NO: 1616 | EEHEE_rd4_0672.pdb |
| SEQ ID NO: 1617 | HHH_rd4_0021.pdb |
| SEQ ID NO: 1618 | EEHEE_rd4_0405.pdb |
| SEQ ID NO: 1619 | EEHEE_rd4_0175.pdb |
| SEQ ID NO: 1620 | EEHEE_rd3_0556.pdb |
| SEQ ID NO: 1621 | EEHEE_rd3_0253.pdb |
| SEQ ID NO: 1622 | HHH_rd4_0753.pdb |
| SEQ ID NO: 1623 | EEHEE_rd3_0525.pdb |
| SEQ ID NO: 1624 | EHEE_rd4_0866.pdb |
| SEQ ID NO: 1625 | HHH_rd4_0311.pdb |
| SEQ ID NO: 1626 | EEHEE_rd4_0383.pdb |
| SEQ ID NO: 1627 | HHH_rd4_0381.pdb |
| SEQ ID NO: 1628 | HHH_rd4_0743.pdb |
| SEQ ID NO: 1629 | EHEE_rd4_0433.pdb |
| SEQ ID NO: 1630 | EHEE_rd2_1285.pdb |
| SEQ ID NO: 1631 | HHH_rd4_0359.pdb |
| SEQ ID NO: 1632 | HHH_rd4_0097.pdb |
| SEQ ID NO: 1633 | EHEE_rd4_0425.pdb |
| SEQ ID NO: 1634 | EHEE_rd4_0134.pdb |
| SEQ ID NO: 1635 | EEHEE_rd3_1061.pdb |
| SEQ ID NO: 1636 | EEHEE_rd4_0143.pdb |
| SEQ ID NO: 1637 | HHH_rd4_0391.pdb |
| SEQ ID NO: 1638 | HHH_rd3_0133.pdb |
| SEQ ID NO: 1639 | HHH_rd4_0608.pdb |
| SEQ ID NO: 1640 | EEHEE_rd3_0022.pdb |
| SEQ ID NO: 1641 | HHH_rd1_0160.pdb |
| SEQ ID NO: 1642 | HHH_rd4_0757.pdb |
| SEQ ID NO: 1643 | HHH_rd4_0974.pdb |
| SEQ ID NO: 1644 | EEHEE_rd4_0566.pdb |
| SEQ ID NO: 1645 | HHH_rd4_0778.pdb |
| SEQ ID NO: 1646 | HHH_rd3_0002.pdb |
| SEQ ID NO: 1647 | EEHEE_rd4_0209.pdb |
| SEQ ID NO: 1648 | EHEE_rd4_0679.pdb |
| SEQ ID NO: 1649 | HHH_rd4_0008.pdb |
| SEQ ID NO: 1650 | HHH_rd4_0526.pdb |
| SEQ ID NO: 1651 | EEHEE_rd4_0743.pdb |
| SEQ ID NO: 1652 | HHH_rd4_0077.pdb |
| SEQ ID NO: 1653 | HHH_rd3_0018.pdb |
| SEQ ID NO: 1654 | EEHEE_rd4_0301.pdb |
| SEQ ID NO: 1655 | EEHEE_rd4_0704.pdb |
| SEQ ID NO: 1656 | HHH_rd4_0986.pdb |
| SEQ ID NO: 1657 | EHEE_rd4_0572.pdb |
| SEQ ID NO: 1658 | HHH_rd4_0539.pdb |
| SEQ ID NO: 1659 | HHH_rd4_0896.pdb |
| SEQ ID NO: 1660 | HHH_rd4_0205.pdb |
| SEQ ID NO: 1661 | HEEH_rd3_0142.pdb |
| SEQ ID NO: 1662 | EEHEE_rd4_0081.pdb |
| SEQ ID NO: 1663 | EEHEE_rd4_0375.pdb |
| SEQ ID NO: 1664 | EEHEE_rd4_0411.pdb |
| SEQ ID NO: 1665 | HHH_rd4_0507.pdb |
| SEQ ID NO: 1666 | EEHEE_rd4_0231.pdb |
| SEQ ID NO: 1667 | HHH_rd4_0357.pdb |
| SEQ ID NO: 1668 | HHH_rd1_0428.pdb |
| SEQ ID NO: 1669 | EHEE_rd4_0813.pdb |
| SEQ ID NO: 1670 | EEHEE_rd4_0369.pdb |
| SEQ ID NO: 1671 | HHH_rd4_0222.pdb |
| SEQ ID NO: 1672 | HHH_rd1_0293.pdb |
| SEQ ID NO: 1673 | EEHEE_rd4_0306.pdb |
| SEQ ID NO: 1674 | EHEE_rd4_0173.pdb |
| SEQ ID NO: 1675 | EEHEE_rd3_0225.pdb |
| SEQ ID NO: 1676 | EHEE_rd3_0053.pdb |
| SEQ ID NO: 1677 | HHH_rd1_0371.pdb |
| SEQ ID NO: 1678 | HHH_rd3_0103.pdb |
| SEQ ID NO: 1679 | EEHEE_rd4_0164.pdb |
| SEQ ID NO: 1680 | EEHEE_rd4_0786.pdb |
| SEQ ID NO: 1681 | HHH_rd3_0045.pdb |
| SEQ ID NO: 1682 | HHH_rd4_0293.pdb |
| SEQ ID NO: 1683 | EHEE_rd4_0671.pdb |
| SEQ ID NO: 1684 | HHH_rd4_0725.pdb |
| SEQ ID NO: 1685 | HHH_rd4_0704.pdb |
| SEQ ID NO: 1686 | EHEE_rd4_0662.pdb |
| SEQ ID NO: 1687 | EEHEE_rd4_0180.pdb |
| SEQ ID NO: 1688 | HHH_rd1_0221.pdb |
| SEQ ID NO: 1689 | HHH_rd4_0198.pdb |
| SEQ ID NO: 1690 | EEHEE_rd4_0801.pdb |
| SEQ ID NO: 1691 | EHEE_rd4_0385.pdb |
| SEQ ID NO: 1692 | EEHEE_rd4_0161.pdb |
| SEQ ID NO: 1693 | EHEE_rd2_0886.pdb |
| SEQ ID NO: 1694 | HHH_rd4_0937.pdb |
| SEQ ID NO: 1695 | EEHEE_rd3_0985.pdb |
| SEQ ID NO: 1696 | EEHEE_rd3_0204.pdb |
| SEQ ID NO: 1697 | HHH_rd4_0803.pdb |
| SEQ ID NO: 1698 | HHH_rd4_0337.pdb |
| SEQ ID NO: 1699 | EEHEE_rd4_0972.pdb |
| SEQ ID NO: 1700 | HHH_rd4_0207.pdb |
| SEQ ID NO: 1701 | EEHEE_rd4_0277.pdb |
| SEQ ID NO: 1702 | EHEE_rd4_0394.pdb |
| SEQ ID NO: 1703 | EHEE_rd3_0035.pdb |
| SEQ ID NO: 1704 | EHEE_rd4_0515.pdb |
| SEQ ID NO: 1705 | EHEE_rd3_0109.pdb |
| SEQ ID NO: 1706 | HHH_rd4_0282.pdb |
| SEQ ID NO: 1707 | HHH_rd1_0692.pdb |
| SEQ ID NO: 1708 | HHH_rd3_0136.pdb |
| SEQ ID NO: 1709 | EHEE_rd4_0332.pdb |
| SEQ ID NO: 1710 | HHH_rd4_0727.pdb |
| SEQ ID NO: 1711 | HHH_rd1_0191.pdb |
| SEQ ID NO: 1712 | EHEE_rd2_0265.pdb |
| SEQ ID NO: 1713 | EEHEE_rd4_0325.pdb |
| SEQ ID NO: 1714 | HHH_rd2_0067.pdb |
| SEQ ID NO: 1715 | EEHEE_rd3_0168.pdb |
| SEQ ID NO: 1716 | EEHEE_rd4_0630.pdb |
| SEQ ID NO: 1717 | EEHEE_rd4_0046.pdb |
| SEQ ID NO: 1718 | EEHEE_rd3_0303.pdb |
| SEQ ID NO: 1719 | HHH_rd4_0413.pdb |
| SEQ ID NO: 1720 | EHEE_rd4_0794.pdb |
| SEQ ID NO: 1721 | HHH_rd3_0216.pdb |
| SEQ ID NO: 1722 | EHEE_rd4_0643.pdb |
| SEQ ID NO: 1723 | EHEE_rd4_0973.pdb |
| SEQ ID NO: 1724 | HHH_rd4_0706.pdb |
| SEQ ID NO: 1725 | EEHEE_rd4_0409.pdb |
| SEQ ID NO: 1726 | EHEE_rd4_0191.pdb |
| SEQ ID NO: 1727 | HHH_rd3_0013.pdb |
| SEQ ID NO: 1728 | HHH_rd4_0614.pdb |
| SEQ ID NO: 1729 | HHH_rd1_0265.pdb |
| SEQ ID NO: 1730 | HHH_rd1_0139.pdb |
| SEQ ID NO: 1731 | EEHEE_rd4_0533.pdb |
| SEQ ID NO: 1732 | HHH_rd4_0767.pdb |
| SEQ ID NO: 1733 | EEHEE_rd3_0051.pdb |
| SEQ ID NO: 1734 | HHH_rd4_0960.pdb |
| SEQ ID NO: 1735 | EEHEE_rd4_0122.pdb |
| SEQ ID NO: 1736 | HHH_rd4_0663.pdb |
| SEQ ID NO: 1737 | EEHEE_rd4_0609.pdb |
| SEQ ID NO: 1738 | EHEE_rd4_0528.pdb |
| SEQ ID NO: 1739 | EEHEE_rd4_0552.pdb |
| SEQ ID NO: 1740 | HHH_rd4_0748.pdb |
| SEQ ID NO: 1741 | EEHEE_rd4_0045.pdb |
| SEQ ID NO: 1742 | HHH_rd1_0062.pdb |
| SEQ ID NO: 1743 | HHH_rd1_0203.pdb |
| SEQ ID NO: 1744 | EEHEE_rd3_1474.pdb |
| SEQ ID NO: 1745 | EHEE_rd4_0464.pdb |
| SEQ ID NO: 1746 | HHH_rd4_0451.pdb |
| SEQ ID NO: 1747 | HHH_rd4_0292.pdb |
| SEQ ID NO: 1748 | EEHEE_rd4_0304.pdb |
| SEQ ID NO: 1749 | EHEE_rd4_0029.pdb |
| SEQ ID NO: 1750 | EEHEE_rd4_0366.pdb |
| SEQ ID NO: 1751 | EEHEE_rd3_1395.pdb |
| SEQ ID NO: 1752 | HHH_rd4_0433.pdb |
| SEQ ID NO: 1753 | EEHEE_rd3_1088.pdb |
| SEQ ID NO: 1754 | HHH_rd4_0633.pdb |
| SEQ ID NO: 1755 | HHH_rd4_0881.pdb |
| SEQ ID NO: 1756 | EEHEE_rd4_0482.pdb |
| SEQ ID NO: 1757 | EEHEE_rd3_0271.pdb |
| SEQ ID NO: 1758 | EEHEE_rd4_0462.pdb |
| SEQ ID NO: 1759 | EHEE_rd4_0254.pdb |
| SEQ ID NO: 1760 | EEHEE_rd4_0479.pdb |
| SEQ ID NO: 1761 | EEHEE_rd3_1600.pdb |
| SEQ ID NO: 1762 | EEHEE_rd4_0860.pdb |
| SEQ ID NO: 1763 | EEHEE_rd3_1582.pdb |
| SEQ ID NO: 1764 | EEHEE_rd4_0388.pdb |
| SEQ ID NO: 1765 | HHH_rd4_0560.pdb |
| SEQ ID NO: 1766 | EHEE_rd1_0559.pdb |
| SEQ ID NO: 1767 | HHH_rd2_0231.pdb |
| SEQ ID NO: 1768 | EEHEE_rd4_0436.pdb |
| SEQ ID NO: 1769 | HHH_rd4_0043.pdb |
| SEQ ID NO: 1770 | HHH_rd1_0517.pdb |
| SEQ ID NO: 1771 | EEHEE_rd3_0210.pdb |
| SEQ ID NO: 1772 | EHEE_rd4_0991.pdb |
| SEQ ID NO: 1773 | EEHEE_rd4_0398.pdb |
| SEQ ID NO: 1774 | HHH_rd2_0083.pdb |
| SEQ ID NO: 1775 | EEHEE_rd4_0267.pdb |
| SEQ ID NO: 1776 | EHEE_rd3_0105.pdb |
| SEQ ID NO: 1777 | HHH_rd4_0002.pdb |
| SEQ ID NO: 1778 | EEHEE_rd3_0146.pdb |
| SEQ ID NO: 1779 | EEHEE_rd3_0021.pdb |
| SEQ ID NO: 1780 | EHEE_rd2_0880.pdb |
| SEQ ID NO: 1781 | HHH_rd4_0973.pdb |
| SEQ ID NO: 1782 | HHH_rd4_0025.pdb |
| SEQ ID NO: 1783 | HHH_rd1_0167.pdb |
| SEQ ID NO: 1784 | HHH_rd4_0015.pdb |
| SEQ ID NO: 1785 | EHEE_rd4_0215.pdb |
| SEQ ID NO: 1786 | HHH_rd4_0236.pdb |
| SEQ ID NO: 1787 | EEHEE_rd4_0321.pdb |
| SEQ ID NO: 1788 | EEHEE_rd4_0238.pdb |
| SEQ ID NO: 1789 | EHEE_rd4_0255.pdb |
| SEQ ID NO: 1790 | EHEE_rd4_0596.pdb |
| SEQ ID NO: 1791 | HHH_rd4_0480.pdb |
| SEQ ID NO: 1792 | EHEE_rd2_1075.pdb |
| SEQ ID NO: 1793 | EEHEE_rd3_0296.pdb |
| SEQ ID NO: 1794 | EEHEE_rd4_0380.pdb |
| SEQ ID NO: 1795 | HHH_rd1_0256.pdb |
| SEQ ID NO: 1796 | EEHEE_rd3_1733.pdb |
| SEQ ID NO: 1797 | EEHEE_rd3_1771.pdb |
| SEQ ID NO: 1798 | EEHEE_rd4_0293.pdb |
| SEQ ID NO: 1799 | EHEE_rd3_0081.pdb |
| SEQ ID NO: 1800 | EEHEE_rd4_0354.pdb |
| SEQ ID NO: 1801 | EEHEE_rd4_0162.pdb |
| SEQ ID NO: 1802 | HEEH_rd3_0128.pdb |
| SEQ ID NO: 1803 | HHH_rd2_0087.pdb |
| SEQ ID NO: 1804 | EEHEE_rd4_0086.pdb |
| SEQ ID NO: 1805 | HHH_rd4_0351.pdb |
| SEQ ID NO: 1806 | EEHEE_rd3_0094.pdb |
| SEQ ID NO: 1807 | EEHEE_rd4_0378.pdb |
| SEQ ID NO: 1808 | HHH_rd4_0033.pdb |
| SEQ ID NO: 1809 | EEHEE_rd4_0371.pdb |
| SEQ ID NO: 1810 | EEHEE_rd4_0403.pdb |
| SEQ ID NO: 1811 | EEHEE_rd3_1436.pdb |
| SEQ ID NO: 1812 | HHH_rd3_0129.pdb |
| SEQ ID NO: 1813 | HHH_rd4_0860.pdb |
| SEQ ID NO: 1814 | EEHEE_rd4_0660.pdb |
| SEQ ID NO: 1815 | EEHEE_rd3_0909.pdb |
| SEQ ID NO: 1816 | EEHEE_rd3_0675.pdb |
| SEQ ID NO: 1817 | HHH_rd4_0755.pdb |
| SEQ ID NO: 1818 | EEHEE_rd4_0692.pdb |
| SEQ ID NO: 1819 | HHH_rd3_0047.pdb |
| SEQ ID NO: 1820 | HHH_rd4_0554.pdb |
| SEQ ID NO: 1821 | EEHEE_rd3_1615.pdb |
| SEQ ID NO: 1822 | HHH_rd1_0554.pdb |
| SEQ ID NO: 1823 | HHH_rd1_0631.pdb |
| SEQ ID NO: 1824 | HHH_rd4_0735.pdb |
| SEQ ID NO: 1825 | HHH_rd4_0968.pdb |
| SEQ ID NO: 1826 | HHH_rd4_0687.pdb |
| SEQ ID NO: 1827 | EHEE_rd4_0829.pdb |
| SEQ ID NO: 1828 | EHEE_rd4_0707.pdb |
| SEQ ID NO: 1829 | EHEE_rd4_0894.pdb |
| SEQ ID NO: 1830 | EEHEE_rd4_0932.pdb |
| SEQ ID NO: 1831 | HHH_rd3_0090.pdb |
| SEQ ID NO: 1832 | EHEE_rd4_0636.pdb |
| SEQ ID NO: 1833 | EEHEE_rd4_0646.pdb |
| SEQ ID NO: 1834 | EEHEE_rd3_1215.pdb |
| SEQ ID NO: 1835 | HHH_rd3_0074.pdb |
| SEQ ID NO: 1836 | EHEE_rd4_0912.pdb |
| SEQ ID NO: 1837 | HEEH_rd3_0713.pdb |
| SEQ ID NO: 1838 | EEHEE_rd4_0986.pdb |
| SEQ ID NO: 1839 | HHH_rd4_0540.pdb |
| SEQ ID NO: 1840 | HHH_rd4_0536.pdb |
| SEQ ID NO: 1841 | HHH_rd4_0871.pdb |
| SEQ ID NO: 1842 | EHEE_rd4_0450.pdb |
| SEQ ID NO: 1843 | EEHEE_rd4_0470.pdb |
| SEQ ID NO: 1844 | EHEE_rd4_0775.pdb |
| SEQ ID NO: 1845 | HEEH_rd3_0002.pdb |
| SEQ ID NO: 1846 | EHEE_rd4_0462.pdb |
| SEQ ID NO: 1847 | HHH_rd4_0889.pdb |
| SEQ ID NO: 1848 | EHEE_rd2_0973.pdb |
| SEQ ID NO: 1849 | EEHEE_rd3_0240.pdb |
| SEQ ID NO: 1850 | HEEH_rd4_0758.pdb |
| SEQ ID NO: 1851 | EEHEE_rd4_0836.pdb |
| SEQ ID NO: 1852 | EEHEE_rd4_0015.pdb |
| SEQ ID NO: 1853 | EHEE_rd2_0165.pdb |
| SEQ ID NO: 1854 | HHH_rd4_0747.pdb |
| SEQ ID NO: 1855 | EEHEE_rd4_0368.pdb |
| SEQ ID NO: 1856 | EHEE_rd4_0093.pdb |
| SEQ ID NO: 1857 | EHEE_rd4_0493.pdb |
| SEQ ID NO: 1858 | EHEE_rd4_0507.pdb |
| SEQ ID NO: 1859 | HHH_rd4_0125.pdb |
| SEQ ID NO: 1860 | HHH_rd1_0602.pdb |
| SEQ ID NO: 1861 | EEHEE_rd4_0748.pdb |
| SEQ ID NO: 1862 | EEHEE_rd4_0147.pdb |
| SEQ ID NO: 1863 | HHH_rd4_0438.pdb |
| SEQ ID NO: 1864 | EHEE_rd4_0533.pdb |
| SEQ ID NO: 1865 | HHH_rd4_0925.pdb |
| SEQ ID NO: 1866 | EEHEE_rd4_0352.pdb |
| SEQ ID NO: 1867 | HHH_rd3_0081.pdb |
| SEQ ID NO: 1868 | EHEE_rd4_0981.pdb |
| SEQ ID NO: 1869 | EHEE_rd2_1257.pdb |
| SEQ ID NO: 1870 | HHH_rd1_0811.pdb |
| SEQ ID NO: 1871 | EHEE_rd4_0590.pdb |
| SEQ ID NO: 1872 | HHH_rd2_0073.pdb |
| SEQ ID NO: 1873 | EHEE_rd4_0889.pdb |
| SEQ ID NO: 1874 | EEHEE_rd4_0100.pdb |
| SEQ ID NO: 1875 | HHH_rd2_0127.pdb |
| SEQ ID NO: 1876 | HHH_rd4_0955.pdb |
| SEQ ID NO: 1877 | EEHEE_rd4_0583.pdb |
| SEQ ID NO: 1878 | EEHEE_rd4_0616.pdb |
| SEQ ID NO: 1879 | HHH_rd3_0078.pdb |
| SEQ ID NO: 1880 | EEHEE_rd3_0062.pdb |
| SEQ ID NO: 1881 | EHEE_rd2_0061.pdb |
| SEQ ID NO: 1882 | EEHEE_rd4_0504.pdb |
| SEQ ID NO: 1883 | HHH_rd1_0288.pdb |
| SEQ ID NO: 1884 | HEEH_rd3_0726.pdb |
| SEQ ID NO: 1885 | EHEE_rd4_0688.pdb |
| SEQ ID NO: 1886 | EEHEE_rd3_0868.pdb |
| SEQ ID NO: 1887 | EEHEE_rd4_0526.pdb |
| SEQ ID NO: 1888 | EHEE_rd4_0883.pdb |
| SEQ ID NO: 1889 | EEHEE_rd4_0307.pdb |
| SEQ ID NO: 1890 | EEHEE_rd4_0138.pdb |
| SEQ ID NO: 1891 | HHH_rd1_0415.pdb |
| SEQ ID NO: 1892 | HHH_rd4_0775.pdb |
| SEQ ID NO: 1893 | HHH_rd1_0866.pdb |
| SEQ ID NO: 1894 | EEHEE_rd4_0578.pdb |
| SEQ ID NO: 1895 | HHH_rd4_0295.pdb |
| SEQ ID NO: 1896 | HHH_rd4_0251.pdb |
| SEQ ID NO: 1897 | EHEE_rd3_0061.pdb |
| SEQ ID NO: 1898 | EEHEE_rd3_0964.pdb |
| SEQ ID NO: 1899 | EHEE_rd4_0855.pdb |
| SEQ ID NO: 1900 | HEEH_rd3_0740.pdb |
| SEQ ID NO: 1901 | HEEH_rd3_0680.pdb |
| SEQ ID NO: 1902 | HHH_rd4_0335.pdb |
| SEQ ID NO: 1903 | HHH_rd3_0071.pdb |
| SEQ ID NO: 1904 | EHEE_rd4_0005.pdb |
| SEQ ID NO: 1905 | HEEH_rd4_0042.pdb |
| SEQ ID NO: 1906 | EEHEE_rd4_0059.pdb |
| SEQ ID NO: 1907 | EEHEE_rd4_0873.pdb |
| SEQ ID NO: 1908 | EHEE_rd3_0130.pdb |
| SEQ ID NO: 1909 | HHH_rd1_0564.pdb |
| SEQ ID NO: 1910 | HHH_rd4_0255.pdb |
| SEQ ID NO: 1911 | EEHEE_rd4_0343.pdb |
| SEQ ID NO: 1912 | EHEE_rd3_0229.pdb |
| SEQ ID NO: 1913 | EEHEE_rd3_1621.pdb |
| SEQ ID NO: 1914 | EEHEE_rd4_0222.pdb |
| SEQ ID NO: 1915 | HHH_rd4_0879.pdb |
| SEQ ID NO: 1916 | HHH_rd4_0458.pdb |
| SEQ ID NO: 1917 | HHH_rd3_0248.pdb |
| SEQ ID NO: 1918 | HHH_rd1_0272.pdb |
| SEQ ID NO: 1919 | HHH_rd4_0953.pdb |
| SEQ ID NO: 1920 | EEHEE_rd4_0428.pdb |
| SEQ ID NO: 1921 | HHH_rd4_0270.pdb |
| SEQ ID NO: 1922 | HHH_rd3_0163.pdb |
| SEQ ID NO: 1923 | EHEE_rd4_0335.pdb |
| SEQ ID NO: 1924 | EHEE_rd4_0047.pdb |
| SEQ ID NO: 1925 | HHH_rd4_0327.pdb |
| SEQ ID NO: 1926 | HEEH_rd2_0127.pdb |
| SEQ ID NO: 1927 | HHH_rd2_0204.pdb |
| SEQ ID NO: 1928 | HHH_rd1_0242.pdb |
| SEQ ID NO: 1929 | HHH_rd2_0236.pdb |
| SEQ ID NO: 1930 | EHEE_rd4_0221.pdb |
| SEQ ID NO: 1931 | EEHEE_rd4_0074.pdb |
| SEQ ID NO: 1932 | EHEE_rd2_0217.pdb |
| SEQ ID NO: 1933 | EEHEE_rd4_0477.pdb |
| SEQ ID NO: 1934 | HHH_rd4_0744.pdb |
| SEQ ID NO: 1935 | EEHEE_rd4_0224.pdb |
| SEQ ID NO: 1936 | EEHEE_rd4_0320.pdb |
| SEQ ID NO: 1937 | EEHEE_rd3_1359.pdb |
| SEQ ID NO: 1938 | EEHEE_rd3_1038.pdb |
| SEQ ID NO: 1939 | HHH_rd4_0863.pdb |
| SEQ ID NO: 1940 | HHH_rd4_0093.pdb |
| SEQ ID NO: 1941 | EEHEE_rd4_0612.pdb |
| SEQ ID NO: 1942 | HHH_rd4_0815.pdb |
| SEQ ID NO: 1943 | EHEE_rd4_0031.pdb |
| SEQ ID NO: 1944 | HHH_rd2_0175.pdb |
| SEQ ID NO: 1945 | HEEH_rd3_0190.pdb |
| SEQ ID NO: 1946 | HHH_rd3_0024.pdb |
| SEQ ID NO: 1947 | EEHEE_rd3_1720.pdb |
| SEQ ID NO: 1948 | EEHEE_rd3_0078.pdb |
| SEQ ID NO: 1949 | HHH_rd1_0306.pdb |
| SEQ ID NO: 1950 | HHH_rd2_0055.pdb |
| SEQ ID NO: 1951 | HHH_rd1_0423.pdb |
| SEQ ID NO: 1952 | EEHEE_rd3_0696.pdb |
| SEQ ID NO: 1953 | EHEE_rd2_1040.pdb |
| SEQ ID NO: 1954 | EHEE_rd4_0377.pdb |
| SEQ ID NO: 1955 | HHH_rd1_0255.pdb |
| SEQ ID NO: 1956 | HHH_rd4_0531.pdb |
| SEQ ID NO: 1957 | HHH_rd4_0522.pdb |
| SEQ ID NO: 1958 | EHEE_rd2_0874.pdb |
| SEQ ID NO: 1959 | EHEE_rd4_0595.pdb |
| SEQ ID NO: 1960 | EHEE_rd2_0972.pdb |
| SEQ ID NO: 1961 | HHH_rd1_0586.pdb |
| SEQ ID NO: 1962 | HHH_rd4_0683.pdb |
| SEQ ID NO: 1963 | HHH_rd4_0701.pdb |
| SEQ ID NO: 1964 | EEHEE_rd4_0051.pdb |
| SEQ ID NO: 1965 | EEHEE_rd4_0562.pdb |
| SEQ ID NO: 1966 | EEHEE_rd4_0576.pdb |
| SEQ ID NO: 1967 | EEHEE_rd4_0112.pdb |
| SEQ ID NO: 1968 | EEHEE_rd4_0624.pdb |
| SEQ ID NO: 1969 | EHEE_rd4_0690.pdb |
| SEQ ID NO: 1970 | EHEE_rd4_0682.pdb |
| SEQ ID NO: 1971 | HHH_rd1_0118.pdb |
| SEQ ID NO: 1972 | HHH_rd3_0236.pdb |
| SEQ ID NO: 1973 | HEEH_rd4_0647.pdb |
| SEQ ID NO: 1974 | EEHEE_rd4_0314.pdb |
| SEQ ID NO: 1975 | EHEE_rd4_0442.pdb |
| SEQ ID NO: 1976 | EEHEE_rd4_0705.pdb |
| SEQ ID NO: 1977 | EEHEE_rd4_0311.pdb |
| SEQ ID NO: 1978 | EEHEE_rd4_0814.pdb |
| SEQ ID NO: 1979 | HHH_rd3_0174.pdb |
| SEQ ID NO: 1980 | EEHEE_rd4_0819.pdb |
| SEQ ID NO: 1981 | EEHEE_rd3_0013.pdb |
| SEQ ID NO: 1982 | EEHEE_rd3_0017.pdb |
| SEQ ID NO: 1983 | HHH_rd3_0130.pdb |
| SEQ ID NO: 1984 | HHH_rd3_0096.pdb |
| SEQ ID NO: 1985 | HHH_rd1_0066.pdb |
| SEQ ID NO: 1986 | EEHEE_rd3_1347.pdb |
| SEQ ID NO: 1987 | EEHEE_rd4_0167.pdb |
| SEQ ID NO: 1988 | HHH_rd4_0911.pdb |
| SEQ ID NO: 1989 | HHH_rd4_0058.pdb |
| SEQ ID NO: 1990 | EHEE_rd4_0750.pdb |
| SEQ ID NO: 1991 | HHH_rd4_0037.pdb |
| SEQ ID NO: 1992 | HHH_rd4_0101.pdb |
| SEQ ID NO: 1993 | HHH_rd1_0684.pdb |
| SEQ ID NO: 1994 | EEHEE_rd3_0987.pdb |
| SEQ ID NO: 1995 | HHH_rd4_0118.pdb |
| SEQ ID NO: 1996 | EHEE_rd4_0727.pdb |
| SEQ ID NO: 1997 | HEEH_rd3_0324.pdb |
| SEQ ID NO: 1998 | EHEE_rd4_0282.pdb |
| SEQ ID NO: 1999 | HHH_rd1_0040.pdb |
| SEQ ID NO: 2000 | EHEE_rd1_0817.pdb |
| SEQ ID NO: 2001 | EEHEE_rd3_0351.pdb |
| SEQ ID NO: 2002 | HHH_rd3_0109.pdb |
| SEQ ID NO: 2003 | EHEE_rd4_0941.pdb |
| SEQ ID NO: 2004 | HHH_rd4_0806.pdb |
| SEQ ID NO: 2005 | HHH_rd1_0674.pdb |
| SEQ ID NO: 2006 | EHEE_rd2_0487.pdb |
| SEQ ID NO: 2007 | HHH_rd1_0450.pdb |
| SEQ ID NO: 2008 | EEHEE_rd3_0907.pdb |
| SEQ ID NO: 2009 | EEHEE_rd4_0590.pdb |
| SEQ ID NO: 2010 | EHEE_rd4_0790.pdb |
| SEQ ID NO: 2011 | EEHEE_rd3_0319.pdb |
| SEQ ID NO: 2012 | HHH_rd4_0720.pdb |
| SEQ ID NO: 2013 | HHH_rd2_0064.pdb |
| SEQ ID NO: 2014 | EEHEE_rd4_0791.pdb |
| SEQ ID NO: 2015 | HHH_rd2_0052.pdb |
| SEQ ID NO: 2016 | HHH_rd3_0119.pdb |
| SEQ ID NO: 2017 | EHEE_rd4_0378.pdb |
| SEQ ID NO: 2018 | EHEE_rd4_0228.pdb |
| SEQ ID NO: 2019 | EHEE_rd2_1106.pdb |
| SEQ ID NO: 2020 | HHH_rd4_0432.pdb |
| SEQ ID NO: 2021 | HEEH_rd4_0024.pdb |
| SEQ ID NO: 2022 | EHEE_rd2_0601.pdb |
| SEQ ID NO: 2023 | HHH_rd4_05347.pdb |
| SEQ ID NO: 2024 | EEHEE_rd4_0426.pdb |
| SEQ ID NO: 2025 | EEHEE_rd4_0413.pdb |
| SEQ ID NO: 2026 | EEHEE_rd4_0467.pdb |
| SEQ ID NO: 2027 | EHEE_rd4_0479.pdb |
| SEQ ID NO: 2028 | HHH_rd4_0884.pdb |
| SEQ ID NO: 2029 | HHH_rd4_0455.pdb |
| SEQ ID NO: 2030 | EEHEE_rd3_0757.pdb |
| SEQ ID NO: 2031 | HHH_rd4_0249.pdb |
| SEQ ID NO: 2032 | HHH_rd4_0545.pdb |
| SEQ ID NO: 2033 | EEHEE_rd3_0650.pdb |
| SEQ ID NO: 2034 | EEHEE_rd3_1570.pdb |
| SEQ ID NO: 2035 | EEHEE_rd3_0709.pdb |
| SEQ ID NO: 2036 | EEHEE_rd4_0843.pdb |
| SEQ ID NO: 2037 | EHEE_rd4_0964.pdb |
| SEQ ID NO: 2038 | HHH_rd4_0971.pdb |
| SEQ ID NO: 2039 | HHH_rd4_0253.pdb |
| SEQ ID NO: 2040 | HHH_rd3_0157.pdb |
| SEQ ID NO: 2041 | HHH_rd4_0823.pdb |
| SEQ ID NO: 2042 | HHH_rd2_0070.pdb |
| SEQ ID NO: 2043 | EEHEE_rd3_0693.pdb |
| SEQ ID NO: 2044 | HHH_rd4_0434.pdb |
| SEQ ID NO: 2045 | EEHEE_rd3_0189.pdb |
| SEQ ID NO: 2046 | HHH_rd3_0126.pdb |
| SEQ ID NO: 2047 | EEHEE_rd3_1324.pdb |
| SEQ ID NO: 2048 | HHH_rd4_0042.pdb |
| SEQ ID NO: 2049 | EHEE_rd4_0904.pdb |
| SEQ ID NO: 2050 | EHEE_rd4_0811.pdb |
| SEQ ID NO: 2051 | EHEE_rd2_0914.pdb |
| SEQ ID NO: 2052 | EEHEE_rd4_0804.pdb |
| SEQ ID NO: 2053 | HHH_rd3_0088.pdb |
| SEQ ID NO: 2054 | HHH_rd4_0721.pdb |
| SEQ ID NO: 2055 | HHH_rd2_0159.pdb |
| SEQ ID NO: 2056 | EEHEE_rd3_1240.pdb |
| SEQ ID NO: 2057 | EEHEE_rd4_0897.pdb |
| SEQ ID NO: 2058 | EHEE_rd4_0642.pdb |
| SEQ ID NO: 2059 | HHH_rd1_0538.pdb |
| SEQ ID NO: 2060 | EEHEE_rd4_0678.pdb |
| SEQ ID NO: 2061 | EEHEE_rd4_0848.pdb |
| SEQ ID NO: 2062 | EHEE_rd4_0114.pdb |
| SEQ ID NO: 2063 | EEHEE_rd4_0191.pdb |
| SEQ ID NO: 2064 | EEHEE_rd4_0883.pdb |
| SEQ ID NO: 2065 | HHH_rd1_0478.pdb |
| SEQ ID NO: 2066 | EEHEE_rd4_0177.pdb |
| SEQ ID NO: 2067 | EHEE_rd4_0606.pdb |
| SEQ ID NO: 2068 | HHH_rd3_0155.pdb |
| SEQ ID NO: 2069 | EEHEE_rd4_0605.pdb |
| SEQ ID NO: 2070 | EEHEE_rd4_0239.pdb |
| SEQ ID NO: 2071 | HHH_rd1_0872.pdb |
| SEQ ID NO: 2072 | EEHEE_rd4_0644.pdb |
| SEQ ID NO: 2073 | EEHEE_rd4_0431.pdb |
| SEQ ID NO: 2074 | EEHEE_rd4_0318.pdb |
| SEQ ID NO: 2075 | EHEE_rd4_0489.pdb |
| SEQ ID NO: 2076 | EHEE_rd4_0414.pdb |
| SEQ ID NO: 2077 | EEHEE_rd4_0997.pdb |
| SEQ ID NO: 2078 | EHEE_rd4_0564.pdb |
| SEQ ID NO: 2079 | EEHEE_rd4_0895.pdb |
| SEQ ID NO: 2080 | EHEE_rd4_0631.pdb |
| SEQ ID NO: 2081 | EHEE_rd4_0089.pdb |
| SEQ ID NO: 2082 | EEHEE_rd4_0310.pdb |
| SEQ ID NO: 2083 | HHH_rd1_0976.pdb |
| SEQ ID NO: 2084 | EEHEE_rd4_0541.pdb |
| SEQ ID NO: 2085 | HHH_rd1_0171.pdb |
| SEQ ID NO: 2086 | HHH_rd2_0188.pdb |
| SEQ ID NO: 2087 | EHEE_rd4_0299.pdb |
| SEQ ID NO: 2088 | EHEE_rd4_0091.pdb |
| SEQ ID NO: 2089 | EEHEE_rd3_0325.pdb |
| SEQ ID NO: 2090 | EHEE_rd4_0112.pdb |
| SEQ ID NO: 2091 | EEHEE_rd3_1492.pdb |
| SEQ ID NO: 2092 | EHEE_rd4_0782.pdb |
| SEQ ID NO: 2093 | HHH_rd2_0213.pdb |
| SEQ ID NO: 2094 | HHH_rd1_0014.pdb |
| SEQ ID NO: 2095 | HHH_rd4_0569.pdb |
| SEQ ID NO: 2096 | HHH_rd4_0988.pdb |
| SEQ ID NO: 2097 | HHH_rd1_0262.pdb |
| SEQ ID NO: 2098 | HHH_rd4_0321.pdb |
| SEQ ID NO: 2099 | HHH_rd4_0659.pdb |
| SEQ ID NO: 2100 | EEHEE_rd3_0379.pdb |
| SEQ ID NO: 2101 | HHH_rd4_0272.pdb |
| SEQ ID NO: 2102 | EEHEE_rd4_0452.pdb |
| SEQ ID NO: 2103 | EHEE_rd4_0022.pdb |
| SEQ ID NO: 2104 | EHEE_rd2_1159.pdb |
| SEQ ID NO: 2105 | HHH_rd1_0031.pdb |
| SEQ ID NO: 2106 | EHEE_rd4_0778.pdb |
| SEQ ID NO: 2107 | HHH_rd4_0963.pdb |
| SEQ ID NO: 2108 | HHH_rd4_0796.pdb |
| SEQ ID NO: 2109 | EHEE_rd4_0262.pdb |
| SEQ ID NO: 2110 | EEHEE_rd4_0114.pdb |
| SEQ ID NO: 2111 | EHEE_rd2_0836.pdb |
| SEQ ID NO: 2112 | HHH_rd3_0084.pdb |
| SEQ ID NO: 2113 | EHEE_rd4_0161.pdb |
| SEQ ID NO: 2114 | HHH_rd4_0632.pdb |
| SEQ ID NO: 2115 | EHEE_rd2_0416.pdb |
| SEQ ID NO: 2116 | HHH_rd4_0039.pdb |
| SEQ ID NO: 2117 | EHEE_rd4_0264.pdb |
| SEQ ID NO: 2118 | EEHEE_rd4_0965.pdb |
| SEQ ID NO: 2119 | HHH_rd3_0182.pdb |
| SEQ ID NO: 2120 | EEHEE_rd4_0601.pdb |
| SEQ ID NO: 2121 | EEHEE_rd4_0227.pdb |
| SEQ ID NO: 2122 | EHEE_rd4_0247.pdb |
| SEQ ID NO: 2123 | EEHEE_rd4_0168.pdb |
| SEQ ID NO: 2124 | HHH_rd4_0527.pdb |
| SEQ ID NO: 2125 | EHEE_rd4_0163.pdb |
| SEQ ID NO: 2126 | EEHEE_rd4_0326.pdb |
| SEQ ID NO: 2127 | HHH_rd3_0189.pdb |
| SEQ ID NO: 2128 | EHEE_rd4_0890.pdb |
| SEQ ID NO: 2129 | EHEE_rd1_0470.pdb |
| SEQ ID NO: 2130 | EEHEE_rd4_0174.pdb |
| SEQ ID NO: 2131 | EEHEE_rd3_0158.pdb |
| SEQ ID NO: 2132 | HHH_rd4_0541.pdb |
| SEQ ID NO: 2133 | HEEH_rd3_0026.pdb |
| SEQ ID NO: 2134 | EEHEE_rd3_0924.pdb |
| SEQ ID NO: 2135 | HEEH_rd3_1177.pdb |
| SEQ ID NO: 2136 | EEHEE_rd4_p882.pdb |
| SEQ ID NO: 2137 | EEHEE_rd4_0817.pdb |
| SEQ ID NO: 2138 | EHEE_rd2_0995.pdb |
| SEQ ID NO: 2139 | EHEE_rd2_0002.pdb |
| SEQ ID NO: 2140 | HHH_rd1_0008.pdb |
| SEQ ID NO: 2141 | HHH_rd1_0709.pdb |
| SEQ ID NO: 2142 | HHH_rd4_0412.pdb |
| SEQ ID NO: 2143 | EEHEE_rd3_0238.pdb |
| SEQ ID NO: 2144 | HHH_rd4_0150.pdb |
| SEQ ID NO: 2145 | EHEE_rd3_0157.pdb |
| SEQ ID NO: 2146 | HHH_rd4_0073.pdb |
| SEQ ID NO: 2147 | EEHEE_rd4_0741.pdb |
| SEQ ID NO: 2148 | EHEE_rd4_0869.pdb |
| SEQ ID NO: 2149 | HEEH_rd2_1175.pdb |
| SEQ ID NO: 2150 | EEHEE_rd4_0955.pdb |
| SEQ ID NO: 2151 | HHH_rd4_0352.pdb |
| SEQ ID NO: 2152 | HHH_rd4_0750.pdb |
| SEQ ID NO: 2153 | EHEE_rd4_0728.pdb |
| SEQ ID NO: 2154 | HHH_rd3_0245.pdb |
| SEQ ID NO: 2155 | HHH_rd4_0149.pdb |
| SEQ ID NO: 2156 | HHH_rd3_0170.pdb |
| SEQ ID NO: 2157 | EEHEE_rd3_1211.pdb |
| SEQ ID NO: 2158 | EEHEE_rd3_1214.pdb |
| SEQ ID NO: 2159 | EHEE_rd4_0074.pdb |
| SEQ ID NO: 2160 | HHH_rd4_0035.pdb |
| SEQ ID NO: 2161 | HHH_rd4_0448.pdb |
| SEQ ID NO: 2162 | HHH_rd3_0042.pdb |
| SEQ ID NO: 2163 | EEHEE_rd4_0721.pdb |
| SEQ ID NO: 2164 | EEHEE_rd4_0088.pdb |
| SEQ ID NO: 2165 | EEHEE_rd3_1103.pdb |
| SEQ ID NO: 2166 | EEHEE_rd3_0628.pdb |
| SEQ ID NO: 2167 | EEHEE_rd3_1121.pdb |
| SEQ ID NO: 2168 | EEHEE_rd3_0035.pdb |
| SEQ ID NO: 2169 | HHH_rd4_0565.pdb |
| SEQ ID NO: 2170 | EEHEE_rd4_0991.pdb |
| SEQ ID NO: 2171 | EHEE_rd2_0065.pdb |
| SEQ ID NO: 2172 | EEHEE_rd3_0713.pdb |
| SEQ ID NO: 2173 | HEEH_rd3_0707.pdb |
| SEQ ID NO: 2174 | EHEE_rd4_0230.pdb |
| SEQ ID NO: 2175 | HHH_rd1_0236.pdb |
| SEQ ID NO: 2176 | HHH_rd4_0115.pdb |
| SEQ ID NO: 2177 | EEHEE_rd4_0695.pdb |
| SEQ ID NO: 2178 | EEHEE_rd4_0573.pdb |
| SEQ ID NO: 2179 | EHEE_rd4_0447.pdb |
| SEQ ID NO: 2180 | HHH_rd4_0254.pdb |
| SEQ ID NO: 2181 | EEHEE_rd4_0263.pdb |
| SEQ ID NO: 2182 | HHH_rd1_0419.pdb |
| SEQ ID NO: 2183 | HHH_rd4_0440.pdb |
| SEQ ID NO: 2184 | HHH_rd4_0279.pdb |
| SEQ ID NO: 2185 | EHEE_rd1_0411.pdb |
| SEQ ID NO: 2186 | EEHEE_rd3_0232.pdb |
| SEQ ID NO: 2187 | HHH_rd4_0126.pdb |
| SEQ ID NO: 2188 | HHH_rd3_0162.pdb |
| SEQ ID NO: 2189 | EEHEE_rd4_0723.pdb |
| SEQ ID NO: 2190 | EEHEE_rd4_0908.pdb |
| SEQ ID NO: 2191 | EEHEE_rd4_0400.pdb |
| SEQ ID NO: 2192 | HHH_rd4_0457.pdb |
| SEQ ID NO: 2193 | HHH_rd4_0542.pdb |
| SEQ ID NO: 2194 | HHH_rd3_0114.pdb |
| SEQ ID NO: 2195 | EEHEE_rd4_0420.pdb |
| SEQ ID NO: 2196 | EEHEE_rd4_0941.pdb |
| SEQ ID NO: 2197 | EHEE_rd2_0297.pdb |
| SEQ ID NO: 2198 | HHH_rd4_0057.pdb |
| SEQ ID NO: 2199 | EEHEE_rd4_0805.pdb |
| SEQ ID NO: 2200 | EEHEE_rd3_0118.pdb |
| SEQ ID NO: 2201 | EHEE_rd4_0817.pdb |
| SEQ ID NO: 2202 | EEHEE_rd3_1241.pdb |
| SEQ ID NO: 2203 | EEHEE_rd4_0713.pdb |
| SEQ ID NO: 2204 | HHH_rd3_0178.pdb |
| SEQ ID NO: 2205 | HHH_rd3_0139.pdb |
| SEQ ID NO: 2206 | EEHEE_rd3_0249.pdb |
| SEQ ID NO: 2207 | EEHEE_rd4_0824.pdb |
| SEQ ID NO: 2208 | EEHEE_rd4_0680.pdb |
| SEQ ID NO: 2209 | EEHEE_rd3_0689.pdb |
| SEQ ID NO: 2210 | EHEE_rd4_0795.pdb |
| SEQ ID NO: 2211 | HHH_rd4_0516.pdb |
| SEQ ID NO: 2212 | HHH_rd4_0577.pdb |
| SEQ ID NO: 2213 | EHEE_rd3_0145.pdb |
| SEQ ID NO: 2214 | EEHEE_rd3_0761.pdb |
| SEQ ID NO: 2215 | HHH_rd1_0261.pdb |
| SEQ ID NO: 2216 | HEEH_rd4_0156.pdb |
| SEQ ID NO: 2217 | EEHEE_rd4_0544.pdb |
| SEQ ID NO: 2218 | EHEE_rd3_0115.pdb |
| SEQ ID NO: 2219 | EEHEE_rd3_1362.pdb |
| SEQ ID NO: 2220 | HHH_rd4_0407.pdb |
| SEQ ID NO: 2221 | EEHEE_rd3_1490.pdb |
| SEQ ID NO: 2222 | HHH_rd3_0043.pdb |
| SEQ ID NO: 2223 | EHEE_rd3_0140.pdb |
| SEQ ID NO: 2224 | HEEH_rd3_0665.pdb |
| SEQ ID NO: 2225 | HHH_rd2_0185.pdb |
| SEQ ID NO: 2226 | HHH_rd1_0029.pdb |
| SEQ ID NO: 2227 | EHEE_rd4_0014.pdb |
| SEQ ID NO: 2228 | HHH_rd2_0043.pdb |
| SEQ ID NO: 2229 | EEHEE_rd4_0629.pdb |
| SEQ ID NO: 2230 | EEHEE_rd3_1415.pdb |
| SEQ ID NO: 2231 | HEEH_rd3_1123.pdb |
| SEQ ID NO: 2232 | HHH_rd4_0764.pdb |
| SEQ ID NO: 2233 | HHH_rd2_0233.pdb |
| SEQ ID NO: 2234 | HHH_rd4_0319.pdb |
| SEQ ID NO: 2235 | HHH_rd4_0641.pdb |
| SEQ ID NO: 2236 | HHH_rd2_0020.pdb |
| SEQ ID NO: 2237 | HHH_rd4_0639.pdb |
| SEQ ID NO: 2238 | HEEH_rd3_0872.pdb |
| SEQ ID NO: 2239 | HHH_rd4_0635.pdb |
| SEQ ID NO: 2240 | HHH_rd4_0888.pdb |
| SEQ ID NO: 2241 | EHEE_rd1_0439.pdb |
| SEQ ID NO: 2242 | EEHEE_rd4_0243.pdb |
| SEQ ID NO: 2243 | EEHEE_rd3_0542.pdb |
| SEQ ID NO: 2244 | EEHEE_rd4_0685.pdb |
| SEQ ID NO: 2245 | EEHEE_rd4_0252.pdb |
| SEQ ID NO: 2246 | EEHEE_rd3_0285.pdb |
| SEQ ID NO: 2247 | EHEE_rd4_0998.pdb |
| SEQ ID NO: 2248 | EEHEE_rd4_0994.pdb |
| SEQ ID NO: 2249 | EEHEE_rd4_1000.pdb |
| SEQ ID NO: 2250 | EHEE_rd4_0001.pdb |
| SEQ ID NO: 2251 | HHH_rd4_0927.pdb |
| SEQ ID NO: 2252 | EEHEE_rd4_0701.pdb |
| SEQ ID NO: 2253 | EEHEE_rd4_0632.pdb |
| SEQ ID NO: 2254 | HHH_rd4_0913.pdb |
| SEQ ID NO: 2255 | EHEE_rd4_0971.pdb |
| SEQ ID NO: 2256 | EHEE_rd4_0020.pdb |
| SEQ ID NO: 2257 | EEHEE_rd3_0687.pdb |
| SEQ ID NO: 2258 | EHEE_rd2_0092.pdb |
| SEQ ID NO: 2259 | EHEE_rd4_0887.pdb |
| SEQ ID NO: 2260 | EHEE_rd4_0024.pdb |
| SEQ ID NO: 2261 | EHEE_rd3_0058.pdb |
| SEQ ID NO: 2262 | EEHEE_rd4_0596.pdb |
| SEQ ID NO: 2263 | HHH_rd4_0573.pdb |
| SEQ ID NO: 2264 | HHH_rd4_0675.pdb |
| SEQ ID NO: 2265 | EEHEE_rd4_0492.pdb |
| SEQ ID NO: 2266 | EEHEE_rd4_0332.pdb |
| SEQ ID NO: 2267 | HHH_rd4_0392.pdb |
| SEQ ID NO: 2268 | EHEE_rd4_0123.pdb |
| SEQ ID NO: 2269 | EEHEE_rd3_0755.pdb |
| SEQ ID NO: 2270 | EEHEE_rd4_0217.pdb |
| SEQ ID NO: 2271 | HHH_rd4_0032.pdb |
| SEQ ID NO: 2272 | EHEE_rd4_0486.pdb |
| SEQ ID NO: 2273 | EHEE_rd1_0350.pdb |
| SEQ ID NO: 2274 | EEHEE_rd4_0771.pdb |
| SEQ ID NO: 2275 | HHH_rd3_0118.pdb |
| SEQ ID NO: 2276 | HHH_rd2_0244.pdb |
| SEQ ID NO: 2277 | EEHEE_rd3_0608.pdb |
| SEQ ID NO: 2278 | HHH_rd4_0112.pdb |
| SEQ ID NO: 2279 | EHEE_rd3_0146.pdb |
| SEQ ID NO: 2280 | EHEE_rd4_0259.pdb |
| SEQ ID NO: 2281 | EEHEE_rd3_0910.pdb |
| SEQ ID NO: 2282 | EHEE_rd3_0067.pdb |
| SEQ ID NO: 2283 | EEHEE_rd4_0508.pdb |
| SEQ ID NO: 2284 | EEHEE_rd4_0502.pdb |
| SEQ ID NO: 2285 | EHEE_rd4_0824.pdb |
| SEQ ID NO: 2286 | EEHEE_rd3_1224.pdb |
| SEQ ID NO: 2287 | EEHEE_rd4_0416.pdb |
| SEQ ID NO: 2288 | EEHEE_rd3_0482.pdb |
| SEQ ID NO: 2289 | HHH_rd4_0294.pdb |
| SEQ ID NO: 2290 | EHEE_rd4_0237.pdb |
| SEQ ID NO: 2291 | EHEE_rd4_0730.pdb |
| SEQ ID NO: 2292 | EEHEE_rd4_0579.pdb |
| SEQ ID NO: 2293 | EEHEE_rd4_0111.pdb |
| SEQ ID NO: 2294 | HHH_rd1_0786.pdb |
| SEQ ID NO: 2295 | EEHEE_rd3_0037.pdb |
| SEQ ID NO: 2296 | HHH_rd4_0459.pdb |
| SEQ ID NO: 2297 | EEHEE_rd4_0101.pdb |
| SEQ ID NO: 2298 | EEHEE_rd4_0422.pdb |
| SEQ ID NO: 2299 | HEEH_rd4_0296.pdb |
| SEQ ID NO: 2300 | HHH_rd2_0022.pdb |
| SEQ ID NO: 2301 | HEEH_rd3_0712.pdb |
| SEQ ID NO: 2302 | EHEE_rd2_0020.pdb |
| SEQ ID NO: 2303 | EEHEE_rd4_0105.pdb |
| SEQ ID NO: 2304 | HHH_rd1_0565.pdb |
| SEQ ID NO: 2305 | HEEH_rd3_0066.pdb |
| SEQ ID NO: 2306 | EHEE_rd2_0867.pdb |
| SEQ ID NO: 2307 | EHEE_rd4_0481.pdb |
| SEQ ID NO: 2308 | EEHEE_rd3_0935.pdb |
| SEQ ID NO: 2309 | EEHEE_rd3_0701.pdb |
| SEQ ID NO: 2310 | HHH_rd2_0157.pdb |
| SEQ ID NO: 2311 | EHEE_rd4_0476.pdb |
| SEQ ID NO: 2312 | HHH_rd3_0154.pdb |
| SEQ ID NO: 2313 | HHH_rd3_0028.pdb |
| SEQ ID NO: 2314 | EEHEE_rd4_0414.pdb |
| SEQ ID NO: 2315 | EHEE_rd4_0820.pdb |
| SEQ ID NO: 2316 | HHH_rd1_0012.pdb |
| SEQ ID NO: 2317 | EEHEE_rd4_0826.pdb |
| SEQ ID NO: 2318 | EEHEE_rd4_0522.pdb |
| SEQ ID NO: 2319 | HEEH_rd3_0094.pdb |
| SEQ ID NO: 2320 | EEHEE_rd3_1461.pdb |
| SEQ ID NO: 2321 | EEHEE_rd3_0699.pdb |
| SEQ ID NO: 2322 | EEHEE_rd4_0136.pdb |
| SEQ ID NO: 2323 | EEHEE_rd4_0188.pdb |
| SEQ ID NO: 2324 | EHEE_rd4_0672.pdb |
| SEQ ID NO: 2325 | EEHEE_rd4_0996.pdb |
| SEQ ID NO: 2326 | EHEE_rd4_0075.pdb |
| SEQ ID NO: 2327 | EHEE_rd4_0581.pdb |
| SEQ ID NO: 2328 | HHH_rd4_0589.pdb |
| SEQ ID NO: 2329 | EEHEE_rd4_0659.pdb |
| SEQ ID NO: 2330 | HHH_rd1_0164.pdb |
| SEQ ID NO: 2331 | HHH_rd4_0789.pdb |
| SEQ ID NO: 2332 | EEHEE_rd4_0339.pdb |
| SEQ ID NO: 2333 | HHH_rd4_0312.pdb |
| SEQ ID NO: 2334 | EHEE_rd4_0216.pdb |
| SEQ ID NO: 2335 | HHH_rd1_0188.pdb |
| SEQ ID NO: 2336 | EHEE_rd4_0067.pdb |
| SEQ ID NO: 2337 | EEHEE_rd4_0602.pdb |
| SEQ ID NO: 2338 | EEHEE_rd4_0716.pdb |
| SEQ ID NO: 2339 | HHH_rd3_0019.pdb |
| SEQ ID NO: 2340 | EHEE_rd4_0026.pdb |
| SEQ ID NO: 2341 | EHEE_rd4_0827.pdb |
| SEQ ID NO: 2342 | EHEE_rd3_0009.pdb |
| SEQ ID NO: 2343 | HHH_rd1_0888.pdb |
| SEQ ID NO: 2344 | HEEH_rd4_0930.pdb |
| SEQ ID NO: 2345 | EHEE_rd2_0942.pdb |
| SEQ ID NO: 2346 | EHEE_rd2_0088.pdb |
| SEQ ID NO: 2347 | EEHEE_rd3_1489.pdb |
| SEQ ID NO: 2348 | EHEE_rd2_1026.pdb |
| SEQ ID NO: 2349 | EEHEE_rd3_0879.pdb |
| SEQ ID NO: 2350 | EEHEE_rd4_0195.pdb |
| SEQ ID NO: 2351 | EHEE_rd4_0357.pdb |
| SEQ ID NO: 2352 | EEHEE_rd3_0956.pdb |
| SEQ ID NO: 2353 | HHH_rd4_0772.pdb |
| SEQ ID NO: 2354 | HHH_rd1_0105.pdb |
| SEQ ID NO: 2355 | EEHEE_rd3_0179.pdb |
| SEQ ID NO: 2356 | EEHEE_rd3_0809.pdb |
| SEQ ID NO: 2357 | HHH_rd4_0316.pdb |
| SEQ ID NO: 2358 | EEHEE_rd4_0169.pdb |
| SEQ ID NO: 2359 | HHH_rd4_0680.pdb |
| SEQ ID NO: 2360 | HHH_rd4_0469.pdb |
| SEQ ID NO: 2361 | HHH_rd1_0361.pdb |
| SEQ ID NO: 2362 | HHH_rd4_0634.pdb |
| SEQ ID NO: 2363 | EEHEE_rd4_0134.pdb |
| SEQ ID NO: 2364 | EEHEE_rd3_0541.pdb |
| SEQ ID NO: 2365 | EHEE_rd4_0422.pdb |
| SEQ ID NO: 2366 | EEHEE_rd4_0151.pdb |
| SEQ ID NO: 2367 | EEHEE_rd4_0424.pdb |
| SEQ ID NO: 2368 | HHH_rd4_0624.pdb |
| SEQ ID NO: 2369 | EEHEE_rd4_0382.pdb |
| SEQ ID NO: 2370 | EEHEE_rd4_0472.pdb |
| SEQ ID NO: 2371 | HHH_rd1_0389.pdb |
| SEQ ID NO: 2372 | EEHEE_rd4_0261.pdb |
| SEQ ID NO: 2373 | HHH_rd1_0739.pdb |
| SEQ ID NO: 2374 | EEHEE_rd4_0129.pdb |
| SEQ ID NO: 2375 | EEHEE_rd4_0960.pdb |
| SEQ ID NO: 2376 | EEHEE_rd4_0179.pdb |
| SEQ ID NO: 2377 | EEHEE_rd3_0555.pdb |
| SEQ ID NO: 2378 | EEHEE_rd3_0782.pdb |
| SEQ ID NO: 2379 | EEHEE_rd4_0118.pdb |
| SEQ ID NO: 2380 | EEHEE_rd4_0540.pdb |
| SEQ ID NO: 2381 | EEHEE_rd4_0202.pdb |
| SEQ ID NO: 2382 | HHH_rd4_0952.pdb |
| SEQ ID NO: 2383 | HEEH_rd2_0771.pdb |
| SEQ ID NO: 2384 | HEEH_rd3_1449.pdb |
| SEQ ID NO: 2385 | HHH_rd1_0241.pdb |
| SEQ ID NO: 2386 | EEHEE_rd4_0755.pdb |
| SEQ ID NO: 2387 | EHEE_rd4_0861.pdb |
| SEQ ID NO: 2388 | EEHEE_rd4_0270.pdb |
| SEQ ID NO: 2389 | EEHEE_rd4_0058.pdb |
| SEQ ID NO: 2390 | HHH_rd2_0103.pdb |
| SEQ ID NO: 2391 | HHH_rd4_0517.pdb |
| SEQ ID NO: 2392 | EEHEE_rd4_0124.pdb |
| SEQ ID NO: 2393 | EEHEE_rd4_0792.pdb |
| SEQ ID NO: 2394 | EEHEE_rd4_0207.pdb |
| SEQ ID NO: 2395 | HEEH_rd3_0013.pdb |
| SEQ ID NO: 2396 | EEHEE_rd3_0358.pdb |
| SEQ ID NO: 2397 | EEHEE_rd4_0089.pdb |
| SEQ ID NO: 2398 | HHH_rd3_0237.pdb |
| SEQ ID NO: 2399 | HHH_rd4_0470.pdb |
| SEQ ID NO: 2400 | HHH_rd2_0033.pdb |
| SEQ ID NO: 2401 | EHEE_rd4_0159.pdb |
| SEQ ID NO: 2402 | HHH_rd4_0647.pdb |
| SEQ ID NO: 2403 | HHH_rd4_0795.pdb |
| SEQ ID NO: 2404 | EHEE_rd4_0697.pdb |
| SEQ ID NO: 2405 | EEHEE_rd4_0658.pdb |
| SEQ ID NO: 2406 | EEHEE_rd4_0276.pdb |
| SEQ ID NO: 2407 | HHH_rd4_0385.pdb |
| SEQ ID NO: 2408 | HHH_rd4_0366.pdb |
| SEQ ID NO: 2409 | EEHEE_rd4_0822.pdb |
| SEQ ID NO: 2410 | EEHEE_rd3_1638.pdb |
| SEQ ID NO: 2411 | HHH_rd1_0356.pdb |
| SEQ ID NO: 2412 | EEHEE_rd3_1485.pdb |
| SEQ ID NO: 2413 | EHEE_rd4_0437.pdb |
| SEQ ID NO: 2414 | EEHEE_rd3_0596.pdb |
| SEQ ID NO: 2415 | HHH_rd1_0181.pdb |
| SEQ ID NO: 2416 | HHH_rd1_0941.pdb |
| SEQ ID NO: 2417 | EEHEE_rd4_0465.pdb |
| SEQ ID NO: 2418 | EEHEE_rd3_0288.pdb |
| SEQ ID NO: 2419 | HHH_rd4_0199.pdb |
| SEQ ID NO: 2420 | EHEE_rd4_0502.pdb |
| SEQ ID NO: 2421 | EEHEE_rd3_0080.pdb |
| SEQ ID NO: 2422 | EEHEE_rd3_0281.pdb |
| SEQ ID NO: 2423 | EHEE_rd2_0055.pdb |
| SEQ ID NO: 2424 | EHEE_rd4_0465.pdb |
| SEQ ID NO: 2425 | HHH_rd1_0169.pdb |
| SEQ ID NO: 2426 | EHEE_rd4_0955.pdb |
| SEQ ID NO: 2427 | HHH_rd4_0824.pdb |
| SEQ ID NO: 2428 | HEEH_rd4_0054.pdb |
| SEQ ID NO: 2429 | EEHEE_rd4_0130.pdb |
| SEQ ID NO: 2430 | HHH_rd4_0762.pdb |
| SEQ ID NO: 2431 | HHH_rd4_0657.p8b |
| SEQ ID NO: 2432 | EEHEE_rd4_0053.pdb |
| SEQ ID NO: 2433 | EEHEE_rd3_0901.pdb |
| SEQ ID NO: 2434 | HEEH_rd4_0649.pdb |
| SEQ ID NO: 2435 | EHEE_rd2_1197.pdb |
| SEQ ID NO: 2436 | HHH_rd4_0379.pdb |
| SEQ ID NO: 2437 | EEHEE_rd3_0007.pdb |
| SEQ ID NO: 2438 | EEHEE_rd4_0454.pdb |
| SEQ ID NO: 2439 | EEHEE_rd4_0091.pdb |
| SEQ ID NO: 2440 | HHH_rd4_0511.pdb |
| SEQ ID NO: 2441 | EHEE_rd4_0358.pdb |
| SEQ ID NO: 2442 | EHEE_rd3_0214.pdb |
| SEQ ID NO: 2443 | EEHEE_rd4_0289.pdb |
| SEQ ID NO: 2444 | EEHEE_rd3_0161.pdb |
| SEQ ID NO: 2445 | EHEE_rd2_0148.pdb |
| SEQ ID NO: 2446 | HHH_rd4_0898.pdb |
| SEQ ID NO: 2447 | HEEH_rd2_0779.pdb |
| SEQ ID NO: 2448 | EHEE_rd4_0723.pdb |
| SEQ ID NO: 2449 | EHEE_rd2_0480.pdb |
| SEQ ID NO: 2450 | HHH_rd4_0114.pdb |
| SEQ ID NO: 2451 | EEHEE_rd4_0766.pdb |
| SEQ ID NO: 2452 | EEHEE_rd4_0096.pdb |
| SEQ ID NO: 2453 | HHH_rd4_0493.pdb |
| SEQ ID NO: 2454 | EHEE_rd4_0057.pdb |
| SEQ ID NO: 2455 | EEHEE_rd4_0956.pdb |
| SEQ ID NO: 2456 | EHEE_rd4_0148.pdb |
| SEQ ID NO: 2457 | EEHEE_rd4_0379.pdb |
| SEQ ID NO: 2458 | HHH_rd3_0191.pdb |
| SEQ ID NO: 2459 | EHEE_rd4_0153.pdb |
| SEQ ID NO: 2460 | HHH_rd1_0113.pdb |
| SEQ ID NO: 2461 | EEHEE_rd4_0478.pdb |
| SEQ ID NO: 2462 | EHEE_rd4_0896.pdb |
| SEQ ID NO: 2463 | HHH_rd4_0426.pdb |
| SEQ ID NO: 2464 | EEHEE_rd3_0033.pdb |
| SEQ ID NO: 2465 | EHEE_rd4_0922.pdb |
| SEQ ID NO: 2466 | EEHEE_rd3_0254.pdb |
| SEQ ID NO: 2467 | EEHEE_rd3_0244.pdb |
| SEQ ID NO: 2468 | EEHEE_rd4_0145.pdb |
| SEQ ID NO: 2469 | EEHEE_rd4_0315.pdb |
| SEQ ID NO: 2470 | HHH_rd4_0989.pdb |
| SEQ ID NO: 2471 | EEHEE_rd4_0839.pdb |
| SEQ ID NO: 2472 | EHEE_rd2_0025.pdb |
| SEQ ID NO: 2473 | EHEE_rd2_0247.pdb |
| SEQ ID NO: 2474 | EEHEE_rd4_0542.pdb |
| SEQ ID NO: 2475 | EHEE_rd4_0207.pdb |
| SEQ ID NO: 2476 | HHH_rd1_0268.pdb |
| SEQ ID NO: 2477 | HHH_rd1_0144.pdb |
| SEQ ID NO: 2478 | EEHEE_rd4_0200.pdb |
| SEQ ID NO: 2479 | EEHEE_rd4_0199.pdb |
| SEQ ID NO: 2480 | EEHEE_rd3_1603.pdb |
| SEQ ID NO: 2481 | EEHEE_rd3_0561.pdb |
| SEQ ID NO: 2482 | EHE_rd4_0587.pdb |
| SEQ ID NO: 2483 | HHH_rd1_0584.pdb |
| SEQ ID NO: 2484 | EEHEE_rd4_0319.pdb |
| SEQ ID NO: 2485 | EHEE_rd4_0226.pdb |
| SEQ ID NO: 2486 | EEHEE_rd4_0793.pdb |
| SEQ ID NO: 2487 | EHEE_rd4_0549.pdb |
| SEQ ID NO: 2488 | EEHEE_rd4_0313.pdb |
| SEQ ID NO: 2489 | EHEE_rd4_0806.pdb |
| SEQ ID NO: 2490 | HHH_rd4_0967.pdb |
| SEQ ID NO: 2491 | EHEE_rd4_0600.pdb |
| SEQ ID NO: 2492 | EHEE_rd4_0085.pdb |
| SEQ ID NO: 2493 | HEEH_rd2_0163.pdb |
| SEQ ID NO: 2494 | EHEE_rd4_0748.pdb |
| SEQ ID NO: 2495 | EEHEE_rd4_0782.pdb |
| SEQ ID NO: 2496 | EEHEE_rd4_0466.pdb |
| SEQ ID NO: 2497 | EEHEE_rd3_1480.pdb |
| SEQ ID NO: 2498 | HHH_rd1_0949.pdb |
| SEQ ID NO: 2499 | EEHEE_rd3_1590.pdb |
| SEQ ID NO: 2500 | EEHEE_rd4_0040.pdb |
| SEQ ID NO: 2501 | HHH_rd4_0623.pdb |
| SEQ ID NO: 2502 | EEHEE_rd4_0073.pdb |
| SEQ ID NO: 2503 | EHEE_rd4_0864.pdb |
| SEQ ID NO: 2504 | EHEE_rd4_0852.pdb |
| SEQ ID NO: 2505 | EEHEE_rd4_0513.pdb |
| SEQ ID NO: 2506 | HHH_rd1_0374.pdb |
| SEQ ID NO: 2507 | HHH_rd4_0668.pdb |
| SEQ ID NO: 2508 | EHEE_rd4_0543.pdb |
| SEQ ID NO: 2509 | EHEE_rd4_0729.pdb |
| SEQ ID NO: 2510 | EEHEE_rd4_0813.pdb |
| SEQ ID NO: 2511 | EHEE_rd4_0113.pdb |
| SEQ ID NO: 2512 | HHH_rd4_0501.pdb |
| SEQ ID NO: 2513 | EEHEE_rd4_0127.pdb |
| SEQ ID NO: 2514 | EEHEE_rd3_1263.pdb |
| SEQ ID NO: 2515 | EEHEE_rd4_0541.pdb |
| SEQ ID NO: 2516 | EHEE_rd4_0752.pdb |
| SEQ ID NO: 2517 | EEHEE_rd4_0110.pdb |
| SEQ ID NO: 2518 | EHEE_rd4_0019.pdb |
| SEQ ID NO: 2519 | HEEH_rd4_0109.pdb |
| SEQ ID NO: 2520 | EHEE_rd4_0936.pdb |
| SEQ ID NO: 2521 | EHEE_rd2_0936.pdb |
| SEQ ID NO: 2522 | EEHEE_rd3_0353.pdb |
| SEQ ID NO: 2523 | EEHEE_rd4_0085.pdb |
| SEQ ID NO: 2524 | HHH_rd4_0376.pdb |
| SEQ ID NO: 2525 | EHEE_rd4_0426.pdb |
| SEQ ID NO: 2526 | HHH_rd4_0441.pdb |
| SEQ ID NO: 2527 | HHH_rd4_0104.pdb |
| SEQ ID NO: 2528 | EEHEE_rd3_1677.pdb |
| SEQ ID NO: 2529 | EEHEE_rd4_0324.pdb |
| SEQ ID NO: 2530 | EEHEE_rd3_1700.pdb |
| SEQ ID NO: 2531 | EEHEE_rd3_0337.pdb |
| SEQ ID NO: 2532 | HHH_rd4_0380.pdb |
| SEQ ID NO: 2533 | HHH_rd4_0106.pdb |
| SEQ ID NO: 2534 | HHH_rd4_0256.pdb |
| SEQ ID NO: 2535 | EHEE_rd4_0401.pdb |
| SEQ ID NO: 2536 | HHH_rd2_0171.pdb |
| SEQ ID NO: 2537 | EEHEE_rd4_0729.pdb |
| SEQ ID NO: 2538 | HHH_rd2_0029.pdb |
| SEQ ID NO: 2539 | EHEE_rd4_0059.pdb |
| SEQ ID NO: 2540 | EHEE_rd4_0056.pdb |
| SEQ ID NO: 2541 | HHH_rd4_0781.pdb |
| SEQ ID NO: 2542 | EEHEE_rd4_0585.pdb |
| SEQ ID NO: 2543 | EHEE_rd4_0472.pdb |
| SEQ ID NO: 2544 | EEHEE_rd4_0280.pdb |
| SEQ ID NO: 2545 | EEHEE_rd4_0230.pdb |
| SEQ ID NO: 2546 | HHH_rd1_0165.pdb |
| SEQ ID NO: 2547 | EEHEE_rd4_0218.pdb |
| SEQ ID NO: 2548 | EEHEE_rd4_0427.pdb |
| SEQ ID NO: 2549 | EEHEE_rd4_0710.pdb |
| SEQ ID NO: 2550 | EEHEE_rd3_1455.pdb |
| SEQ ID NO: 2551 | EEHEE_rd4_0683.pdb |
| SEQ ID NO: 2552 | EHEE_rd4_0625.pdb |
| SEQ ID NO: 2553 | EHEE_rd4_0129.pdb |
| SEQ ID NO: 2554 | EEHEE_rd4_0357.pdb |
| SEQ ID NO: 2555 | EEHEE_rd4_0442.pdb |
| SEQ ID NO: 2556 | HHH_rd4_0435.pdb |
| SEQ ID NO: 2557 | EEHEE_rd4_0469.pdb |
| SEQ ID NO: 2558 | HEEH_rd4_0398.pdb |
| SEQ ID NO: 2559 | EEHEE_rd3_1731.pdb |
| SEQ ID NO: 2560 | EHEE_rd4_0816.pdb |
| SEQ ID NO: 2561 | HHH_rd3_0134.pdb |
| SEQ ID NO: 2562 | EHEE_rd4_0408.pdb |
| SEQ ID NO: 2563 | HHH_rd1_0275.pdb |
| SEQ ID NO: 2564 | EEHEE_rd3_1196.pdb |
| SEQ ID NO: 2565 | HHH_rd4_0978.pdb |
| SEQ ID NO: 2566 | EEHEE_rd3_0592.pdb |
| SEQ ID NO: 2567 | HHH_rd2_0006.pdb |
| SEQ ID NO: 2568 | EHEE_rd3_0223.pdb |
| SEQ ID NO: 2569 | EEHEE_rd3_0313.pdb |
| SEQ ID NO: 2570 | EEHEE_rd3_1336.pdb |
| SEQ ID NO: 2571 | HHH_rd1_0773.pdb |
| SEQ ID NO: 2572 | EHEE_rd2_0920.pdb |
| SEQ ID NO: 2573 | EHEE_rd4_0649.pdb |
| SEQ ID NO: 2574 | EHEE_rd4_0231.pdb |
| SEQ ID NO: 2575 | EEHEE_rd4_0661.pdb |
| SEQ ID NO: 2576 | HHH_rd4_0751.pdb |
| SEQ ID NO: 2577 | EHEE_rd2_0983.pdb |
| SEQ ID NO: 2578 | EHEE_rd2_0450.pdb |
| SEQ ID NO: 2579 | EHEE_rd4_0633.pdb |
| SEQ ID NO: 2580 | EEHEE_rd3_0135.pdb |
| SEQ ID NO: 2581 | EEHEE_rd3_1072.pdb |
| SEQ ID NO: 2582 | HHH_rd1_0863.pdb |
| SEQ ID NO: 2583 | EEHEE_rd4_0510.pdb |
| SEQ ID NO: 2584 | EEHEE_rd4_0585.pdb |
| SEQ ID NO: 2585 | EHEE_rd4_0310.pdb |
| SEQ ID NO: 2586 | EEHEE_rd3_0003.pdb |
| SEQ ID NO: 2587 | HHH_rd3_0202.pdb |
| SEQ ID NO: 2588 | HEEH_rd3_1573.pdb |
| SEQ ID NO: 2589 | EEHEE_rd3_0803.pdb |
| SEQ ID NO: 2590 | EHEE_rd4_0236.pdb |
| SEQ ID NO: 2591 | EHEE_rd4_0217.pdb |
| SEQ ID NO: 2592 | EHEE_rd4_0302.pdb |
| SEQ ID NO: 2593 | EHEE_rd4_0345.pdb |
| SEQ ID NO: 2594 | HHH_rd1_0121.pdb |
| SEQ ID NO: 2595 | HEEH_rd3_1183.pdb |
| SEQ ID NO: 2596 | EEHEE_rd4_0299.pdb |
| SEQ ID NO: 2597 | EEHEE_rd4_0524.pdb |
| SEQ ID NO: 2598 | HHH_rd1_0489.pdb |
| SEQ ID NO: 2599 | EEHEE_rd3_1158.pdb |
| SEQ ID NO: 2600 | HHH_rd1_0982.pdb |
| SEQ ID NO: 2601 | EEHEE_rd3_1803.pdb |
| SEQ ID NO: 2602 | EHEE_rd4_0133.pdb |
| SEQ ID NO: 2603 | EHEE_rd2_1118.pdb |
| SEQ ID NO: 2604 | HHH_rd4_0949.pdb |
| SEQ ID NO: 2605 | EHEE_rd4_0859.pdb |
| SEQ ID NO: 2606 | EEHEE_rd4_0083.pdb |
| SEQ ID NO: 2607 | HEEH_rd3_1011.pdb |
| SEQ ID NO: 2608 | EEHEE_rd3_0435.pdb |
| SEQ ID NO: 2609 | EHEE_rd2_0183.pdb |
| SEQ ID NO: 2610 | EEHEE_rd3_0865.pdb |
| SEQ ID NO: 2611 | EHEE_rd2_0086.pdb |
| SEQ ID NO: 2612 | HHH_rd4_0119.pdb |
| SEQ ID NO: 2613 | HHH_rd4_0607.pdb |
| SEQ ID NO: 2614 | HHH_rd4_0598.pdb |
| SEQ ID NO: 2615 | EEHEE_rd4_0523.pdb |
| SEQ ID NO: 2616 | EEHEE_rd4_0288.pdb |
| SEQ ID NO: 2617 | HHH_rd4_0026.pdb |
| SEQ ID NO: 2618 | HHH_rd4_0555.pdb |
| SEQ ID NO: 2619 | EEHEE_rd3_0801.pdb |
| SEQ ID NO: 2620 | HHH_rd1_0417.pdb |
| SEQ ID NO: 2621 | EHEE_rd4_0905.pdb |
| SEQ ID NO: 2622 | EEHEE_rd3_1141.pdb |
| SEQ ID NO: 2623 | EHEE_rd3_0234.pdb |
| SEQ ID NO: 2624 | HEEH_rd3_0435.pdb |
| SEQ ID NO: 2625 | EEHEE_rd3_0417.pdb |
| SEQ ID NO: 2626 | HHH_rd4_0105.pdb |
| SEQ ID NO: 2627 | HNH_rd3_0131.pdb |
| SEQ ID NO: 2628 | EEHEE_rd4_0599.pdb |
| SEQ ID NO: 2629 | EEHEE_rd4_0608.pdb |
| SEQ ID NO: 2630 | HHH_rd1_0471.pdb |
| SEQ ID NO: 2631 | EEHEE_rd4_0756.pdb |
| SEQ ID NO: 2632 | HHH_rd4_0246.pdb |
| SEQ ID NO: 2633 | EEHEE_rd3_1246.pdb |
| SEQ ID NO: 2634 | EEHEE_rd4_0117.pdb |
| SEQ ID NO: 2635 | EEHEE_rd3_1113.pdb |
| SEQ ID NO: 2636 | EHEE_rd4_0438.pdb |
| SEQ ID NO: 2637 | HHH_rd1_0354.pdb |
| SEQ ID NO: 2638 | EEHEE_rd4_0861.pdb |
| SEQ ID NO: 2639 | EEHEE_rd4_0655.pdb |
| SEQ ID NO: 2640 | EHEE_rd4_0179.pdb |
| SEQ ID NO: 2641 | EEHEE_rd4_0425.pdb |
| SEQ ID NO: 2642 | HHH_rd1_0325.pdb |
| SEQ ID NO: 2643 | EHEE_rd4_0886.pdb |
| SEQ ID NO: 2644 | EHEE_rd2_1151.pdb |
| SEQ ID NO: 2645 | EEHEE_rd3_0895.pdb |
| SEQ ID NO: 2646 | EEHEE_rd4_0377.pdb |
| SEQ ID NO: 2647 | HEEH_rd3_0821.pdb |
| SEQ ID NO: 2648 | EEHEE_rd3_1445.pdb |
| SEQ ID NO: 2649 | HHH_rd1_0299.pdb |
| SEQ ID NO: 2650 | HHH_rd4_0604.pdb |
| SEQ ID NO: 2651 | EHEE_rd4_0550.pdb |
| SEQ ID NO: 2652 | EHEE_rd4_0201.pdb |
| SEQ ID NO: 2653 | HEEH_rd3_0127.pdb |
| SEQ ID NO: 2654 | EHEE_rd3_0005.pdb |
| SEQ ID NO: 2655 | HHH_rd1_0323.pdb |
| SEQ ID NO: 2656 | EEHEE_rd4_0158.pdb |
| SEQ ID NO: 2657 | HHH_rd4_0464.pdb |
| SEQ ID NO: 2658 | EHEE_rd4_0171.pdb |
| SEQ ID NO: 2659 | EHEE_rd2_p650.pdb |
| SEQ ID NO: 2660 | EHEE_rd2_0585.pdb |
| SEQ ID NO: 2661 | EEHEE_rd4_0611.pdb |
| SEQ ID NO: 2662 | EEHEE_rd4_0613.pdb |
| SEQ ID NO: 2663 | EEHEE_rd4_0052.pdb |
| SEQ ID NO: 2664 | HHH_rd3_0241.pdb |
| SEQ ID NO: 2665 | EEHEE_rd4_0432.pdb |
| SEQ ID NO: 2666 | EEHEE_rd3_0489.pdb |
| SEQ ID NO: 2667 | EEHEE_rd3_0260.pdb |
| SEQ ID NO: 2668 | HHH_rd4_0059.pdb |
| SEQ ID NO: 2669 | HHH_rd1_0005.pdb |
| SEQ ID NO: 2670 | HHH_rd1_0104.pdb |
| SEQ ID NO: 2671 | EEHEE_rd4_0924.pdb |
| SEQ ID NO: 2672 | HHH_rd4_0891.pdb |
| SEQ ID NO: 2673 | HHH_rd4_0078.pdb |
| SEQ ID NO: 2674 | HHH_rd4_0156.pdb |
| SEQ ID NO: 2675 | HHH_rd4_0942.pdb |
| SEQ ID NO: 2676 | HHH_rd4_0932.pdb |
| SEQ ID NO: 2677 | EEHEE_rd4_0871.pdb |
| SEQ ID NO: 2678 | EHEE_rd4_0754.pdb |
| SEQ ID NO: 2679 | HHH_rd1_0108.pdb |
| SEQ ID NO: 2680 | EHEE_rd4_0684.pdb |
| SEQ ID NO: 2681 | EEHEE_rd4_0736.pdb |
| SEQ ID NO: 2682 | HHH_rd4_0497.pdb |
| SEQ ID NO: 2683 | EEHEE_rd3_0132.pdb |
| SEQ ID NO: 2684 | EHEE_rd2_0336.pdb |
| SEQ ID NO: 2685 | EHEE_rd2_0763.pdb |
| SEQ ID NO: 2686 | EEHEE_rd3_0096.pdb |
| SEQ ID NO: 2687 | EEHEE_rd4_0988.pdb |
| SEQ ID NO: 2688 | HHH_rd3_0152.pdb |
| SEQ ID NO: 2689 | EEHEE_rd4_0978.pdb |
| SEQ ID NO: 2690 | EEHEE_rd4_0323.pdb |
| SEQ ID NO: 2691 | EHEE_rd4_0658.pdb |
| SEQ ID NO: 2692 | EEHEE_rd3_1377.pdb |
| SEQ ID NO: 2693 | HHH_rd1_0716.pdb |
| SEQ ID NO: 2694 | HHH_rd1_0127.pdb |
| SEQ ID NO: 2695 | EHEE_rd2_0438.pdb |
| SEQ ID NO: 2696 | EEHEE_rd4_0982.pdb |
| SEQ ID NO: 2697 | EEHEE_rd4_0980.pdb |
| SEQ ID NO: 2698 | HHH_rd4_0791.pdb |
| SEQ ID NO: 2699 | HHH_rd1_0196.pdb |
| SEQ ID NO: 2700 | EHEE_rd4_0599.pdb |
| SEQ ID NO: 2701 | HHH_rd1_0080.pdb |
| SEQ ID NO: 2702 | EEHEE_rd3_0933.pdb |
| SEQ ID NO: 2703 | EEHEE_rd4_0635.pdb |
| SEQ ID NO: 2704 | EHEE_rd4_0970_pdb |
| SEQ ID NO: 2705 | EEHEE_rd3_1500.pdb |
| SEQ ID NO: 2706 | HHH_rd4_0944.pdb |
| SEQ ID NO: 2707 | EHEE_rd4_0548.pdb |
| SEQ ID NO: 2708 | EHEE_rd4_0350.pdb |
| SEQ ID NO: 2709 | EEHEE_rd3_1278.pdb |
| SEQ ID NO: 2710 | EHEE_rd4_0831.pdb |
| SEQ ID NO: 2711 | EEHEE_rd4_0360.pdb |
| SEQ ID NO: 2712 | HEEH_rd3_0525.pdb |
| SEQ ID NO: 2713 | EHEE_rd4_0115.pdb |
| SEQ ID NO: 2714 | EHEE_rd4_0182.pdb |
| SEQ ID NO: 2715 | EEHEE_rd4_0939.pdb |
| SEQ ID NO: 2716 | EHEE_rd4_0119.pdb |
| SEQ ID NO: 2717 | EEHEE_rd4_0282.pdb |
| SEQ ID NO: 2718 | HEEH_rd3_0117.pdb |
| SEQ ID NO: 2719 | EHEE_rd2_0222.pdb |
| SEQ ID NO: 2720 | EEHEE_rd4_0395.pdb |
| SEQ ID NO: 2721 | EEHEE_rd4_0421.pdb |
| SEQ ID NO: 2722 | EHEE_rd4_0454.pdb |
| SEQ ID NO: 2723 | EEHEE_rd4_0774.pdb |
| SEQ ID NO: 2724 | EEHEE_rd3_0711.pdb |
| SEQ ID NO: 2725 | HHH_rd1_0421.pdb |
| SEQ ID NO: 2726 | HHH_rd1_0295.pdb |
| SEQ ID NO: 2727 | EHEE_rd4_0753.pdb |
| SEQ ID NO: 2728 | HHH_rd2_0207.pdb |
| SEQ ID NO: 2729 | EEHEE_rd3_0009.pdb |
| SEQ ID NO: 2730 | EHEE_rd4_0311.pdb |
| SEQ ID NO: 2731 | HHH_rd1_0004.pdb |
| SEQ ID NO: 2732 | EHEE_rd2_1127.pdb |
| SEQ ID NO: 2733 | EEHEE_rd4_0265.pdb |
| SEQ ID NO: 2734 | EEHEE_rd3_0045.pdb |
| SEQ ID NO: 2735 | EHEE_rd4_0669.pdb |
| SEQ ID NO: 2736 | EEHEE_rd3_0129.pdb |
| SEQ ID NO: 2737 | HHH_rd3_0101.pdb |
| SEQ ID NO: 2738 | HEEH_rd3_1205.pdb |
| SEQ ID NO: 2739 | HHH_rd2_0062.pdb |
| SEQ ID NO: 2740 | EHEE_rd4_0934.pdb |
| SEQ ID NO: 2741 | HEEH_rd2_0525.pdb |
| SEQ ID NO: 2742 | EHEE_rd2_1108.pdb |
| SEQ ID NO: 2743 | EHEE_rd4_0851.pdb |
| SEQ ID NO: 2744 | EHEE_rd4_0300.pdb |
| SEQ ID NO: 2745 | EHEE_rd4_0818.pdb |
| SEQ ID NO: 2746 | EHEE_rd4_0588.pdb |
| SEQ ID NO: 2747 | EHEE_rd4_0538.pdb |
| SEQ ID NO: 2748 | HHH_rd4_1000.pdb |
| SEQ ID NO: 2749 | EHEE_rd4_0484.pdb |
| SEQ ID NO: 2750 | HEEH_rd4_0872.pdb |
| SEQ ID NO: 2751 | EHEE_rd4_0035.pdb |
| SEQ ID NO: 2752 | EEHEE_rd4_0515.pdb |
| SEQ ID NO: 2753 | EEHEE_rd3_0657.pdb |
| SEQ ID NO: 2754 | HHH_rd4_0681.pdb |
| SEQ ID NO: 2755 | EEHEE_rd3_1375.pdb |
| SEQ ID NO: 2756 | HHH_rd1_0829.pdb |
| SEQ ID NO: 2757 | EEHEE_rd3_1381.pdb |
| SEQ ID NO: 2758 | HHH_rd1_0043.pdb |
| SEQ ID NO: 2759 | HHH_rd4_0842.pdb |
| SEQ ID NO: 2760 | EEHEE_rd3_0830.pdb |
| SEQ ID NO: 2761 | EEHEE_rd3_0590.pdb |
| SEQ ID NO: 2762 | EEHEE_rd3_1418.pdb |
| SEQ ID NO: 2763 | EEHEE_rd4_0863.pdb |
| SEQ ID NO: 2764 | EEHEE_rd4_0589.pdb |
| SEQ ID NO: 2765 | EHEE_rd4_0046.pdb |
| SEQ ID NO: 2766 | HHH_rd4_0919.pdb |
| SEQ ID NO: 2767 | HHH_rd1_0637.pdb |
| SEQ ID NO: 2768 | EEHEE_rd4_0557.pdb |
| SEQ ID NO: 2769 | HHH_rd4_0350.pdb |
| SEQ ID NO: 2770 | EEHEE_rd3_1323.pdb |
| SEQ ID NO: 2771 | HHH_rd1_0207.pdb |
| SEQ ID NO: 2772 | EEHEE_rd4_0802.pdb |
| SEQ ID NO: 2773 | EEHEE_rd4_0512.pdb |
| SEQ ID NO: 2774 | EEHEE_rd4_0983.pdb |
| SEQ ID NO: 2775 | HHH_rd1_0403.pdb |
| SEQ ID NO: 2776 | HHH_rd1_0073.pdb |
| SEQ ID NO: 2777 | HHH_rd4_0509.pdb |
| SEQ ID NO: 2778 | HHH_rd1_0054.pdb |
| SEQ ID NO: 2779 | EEHEE_rd4_0615.pdb |
| SEQ ID NO: 2780 | EHEE_rd4_0670.pdb |
| SEQ ID NO: 2781 | EHEE_rd4_0323.pdb |
| SEQ ID NO: 2782 | EEHEE_rd4_0551.pdb |
| SEQ ID NO: 2783 | EEHEE_rd3_1191.pdb |
| SEQ ID NO: 2784 | HHH_rd4_0484.pdb |
| SEQ ID NO: 2785 | EHEE_rd4_0210.pdb |
| SEQ ID NO: 2786 | EEHEE_rd4_0483.pdb |
| SEQ ID NO: 2787 | HHH_rd4_0063.pdb |
| SEQ ID NO: 2788 | HHH_rd1_0217.pdb |
| SEQ ID NO: 2789 | EEHEE_rd4_0344.pdb |
| SEQ ID NO: 2790 | EHEE_rd4_0675.pdb |
| SEQ ID NO: 2791 | EHEE_rd4_0272.pdb |
| SEQ ID NO: 2792 | HHH_rd1_0329.pdb |
| SEQ ID NO: 2793 | HEEH_rd3_1152.pdb |
| SEQ ID NO: 2794 | EHEE_rd4_0872.pdb |
| SEQ ID NO: 2795 | EEHEE_rd4_0518.pdb |
| SEQ ID NO: 2796 | HHH_rd2_0122.pdb |
| SEQ ID NO: 2797 | HHH_rd3_0027.pdb |
| SEQ ID NO: 2798 | HHH_rd4_0900.pdb |
| SEQ ID NO: 2799 | HHH_rd1_0291.pdb |
| SEQ ID NO: 2800 | EHEE_rd3_0052.pdb |
| SEQ ID NO: 2801 | HHH_rd1_0464.pdb |
| SEQ ID NO: 2802 | EEHEE_rd4_0516.pdb |
| SEQ ID NO: 2803 | HHH_rd2_0024.pdb |
| SEQ ID NO: 2804 | EEHEE_rd3_0776.pdb |
| SEQ ID NO: 2805 | HHH_rd3_0057.pdb |
| SEQ ID NO: 2806 | EHEE_rd2_0706.pdb |
| SEQ ID NO: 2807 | HHH_rd4_0520.pdb |
| SEQ ID NO: 2808 | EEHEE_rd3_0612.pdb |
| SEQ ID NO: 2809 | HEEH_rd3_0473.pdb |
| SEQ ID NO: 2810 | HHH_rd4_0229.pdb |
| SEQ ID NO: 2811 | HHH_rd3_0145.pdb |
| SEQ ID NO: 2812 | EHEE_rd2_0907.pdb |
| SEQ ID NO: 2813 | HEEH_rd3_1304.pdb |
| SEQ ID NO: 2814 | EEHEE_rd4_0864.pdb |
| SEQ ID NO: 2815 | EEHEE_rd3_0258.pdb |
| SEQ ID NO: 2816 | EEHEE_rd3_0870.pdb |
| SEQ ID NO: 2817 | HHH_rd4_0144.pdb |
| SEQ ID NO: 2818 | HHH_rd4_0515.pdb |
| SEQ ID NO: 2819 | EEHEE_rd3_0950.pdb |
| SEQ ID NO: 2820 | EHEE_rd4_0554.pdb |
| SEQ ID NO: 2821 | EEHEE_rd3_0164.pdb |
| SEQ ID NO: 2822 | HHH_rd1_0020.pdb |
| SEQ ID NO: 2823 | EHEE_rd4_0402.pdb |
| SEQ ID NO: 2824 | EEHEE_rd3_0430.pdb |
| SEQ ID NO: 2825 | EEHEE_rd4_0393.pdb |
| SEQ ID NO: 2826 | HHH_rd4_0997.pdb |
| SEQ ID NO: 2827 | EHEE_rd3_0050.pdb |
| SEQ ID NO: 2828 | EHEE_rd4_0388.pdb |
| SEQ ID NO: 2829 | HHH_rd4_0784.pdb |
| SEQ ID NO: 2830 | EEHEE_rd4_0361.pdb |
| SEQ ID NO: 2831 | EEHEE_rd3_1631.pdb |
| SEQ ID NO: 2832 | EHEE_rd4_0375.pdb |
| SEQ ID NO: 2833 | HEEH_rd2_0383.pdb |
| SEQ ID NO: 2834 | HHH_rd1_0101.pdb |
| SEQ ID NO: 2835 | EHEE_rd4_0110.pdb |
| SEQ ID NO: 2836 | HEEH_rd3_1530.pdb |
| SEQ ID NO: 2837 | EHEE_rd4_0293.pdb |
| SEQ ID NO: 2838 | EHEE_rd4_0706.pdb |
| SEQ ID NO: 2839 | EEHEE_rd3_0746.pdb |
| SEQ ID NO: 2840 | HHH_rd1_0117.pdb |
| SEQ ID NO: 2841 | HHH_rd4_0650.pdb |
| SEQ ID NO: 2842 | EEHEE_rd3_0348.pdb |
| SEQ ID NO: 2843 | HHH_rd3_0167.pdb |
| SEQ ID NO: 2844 | HHH_rd4_0843.pdb |
| SEQ ID NO: 2845 | EHEE_rd4_0954.pdb |
| SEQ ID NO: 2846 | EEHEE_rd3_1279.pdb |
| SEQ ID NO: 2847 | HHH_rd1_0522.pdb |
| SEQ ID NO: 2848 | HHH_rd1_0243.pdb |
| SEQ ID NO: 2849 | HHH_rd4_0627.pdb |
| SEQ ID NO: 2850 | HHH_rd4_0261.pdb |
| SEQ ID NO: 2851 | HHH_rd4_0290.pdb |
| SEQ ID NO: 2852 | EEHEE_rd4_0593.pdb |
| SEQ ID NO: 2853 | EEHEE_rd4_0244.pdb |
| SEQ ID NO: 2854 | HEEH_rd2_0360.pdb |
| SEQ ID NO: 2855 | HEEH_rd3_0804.pdb |
| SEQ ID NO: 2856 | EHEE_rd4_0122.pdb |
| SEQ ID NO: 2857 | EEHEE_rd3_1008.pdb |
| SEQ ID NO: 2858 | HHH_rd1_0551.pdb |
| SEQ ID NO: 2859 | HHH_rd3_0046.pdb |
| SEQ ID NO: 2860 | EHEE_rd4_0225.pdb |
| SEQ ID NO: 2861 | EEHEE_rd2_0391.pdb |
| SEQ ID NO: 2862 | HHH_rd1_0225.pdb |
| SEQ ID NO: 2863 | HHH_rd4_0318.pdb |
| SEQ ID NO: 2864 | HHH_rd4_0856.pdb |
| SEQ ID NO: 2865 | EEHEE_rd3_1009.pdb |
| SEQ ID NO: 2866 | HEEH_rd2_0519.pdb |
| SEQ ID NO: 2867 | HHH_rd1_0587.pdb |
| SEQ ID NO: 2868 | EEHEE_rd4_0878.pdb |
| SEQ ID NO: 2869 | EEHEE_rd4_0509.pdb |
| SEQ ID NO: 2870 | EHEE_rd4_0238.pdb |
| SEQ ID NO: 2871 | EHEE_rd2_0794.pdb |
| SEQ ID NO: 2872 | HHH_rd1_0759.pdb |
| SEQ ID NO: 2873 | EHEE_rd4_0914.pdb |
| SEQ ID NO: 2874 | EEHEE_rd4_0892.pdb |
| SEQ ID NO: 2875 | EHEE_rd2_0303.pdb |
| SEQ ID NO: 2876 | EEHEE_rd4_0811.pdb |
| SEQ ID NO: 2877 | EEHEE_rd3_0760.pdb |
| SEQ ID NO: 2878 | EHEE_rd4_0055.pdb |
| SEQ ID NO: 2879 | HEEH_rd3_1547.pdb |
| SEQ ID NO: 2880 | EHEE_rd4_0602.pdb |
| SEQ ID NO: 2881 | HHH_rd1_0535.pdb |
| SEQ ID NO: 2882 | EEHEE_rd3_0181.pdb |
| SEQ ID NO: 2883 | EHEE_rd4_0994.pdb |
| SEQ ID NO: 2884 | EHEE_rd4_0268.pdb |
| SEQ ID NO: 2885 | HHH_rd3_0156.pdb |
| SEQ ID NO: 2886 | EHEE_rd4_0222.pdb |
| SEQ ID NO: 2887 | EHEE_rd4_0787.pdb |
| SEQ ID NO: 2888 | HHH_rd1_0024.pdb |
| SEQ ID NO: 2889 | EEHEE_rd3_0226.pdb |
| SEQ ID NO: 2890 | EHEE_rd2_0941.pdb |
| SEQ ID NO: 2891 | EEHEE_rd4_0545.pdb |
| SEQ ID NO: 2892 | EHEE_rd4_0847.pdb |
| SEQ ID NO: 2893 | EEHEE_rd4_0539.pdb |
| SEQ ID NO: 2894 | EHEE_rd4_0932.pdb |
| SEQ ID NO: 2895 | EEHEE_rd3_0453.pdb |
| SEQ ID NO: 2896 | HEEH_rd2_0539.pdb |
| SEQ ID NO: 2897 | EEHEE_rd4_0761.pdb |
| SEQ ID NO: 2898 | EHEE_rd3_0197.pdb |
| SEQ ID NO: 2899 | HHH_rd4_0876.pdb |
| SEQ ID NO: 2900 | EEHEE_rd4_0412.pdb |
| SEQ ID NO: 2901 | HHH_rd1_0245.pdb |
| SEQ ID NO: 2902 | HHH_rd1_0324.pdb |
| SEQ ID NO: 2903 | EEHEE_rd4_0211.pdb |
| SEQ ID NO: 2904 | EEHEE_rd4_0148.pdb |
| SEQ ID NO: 2905 | HHH_rd4_0886.pdb |
| SEQ ID NO: 2906 | HHH_rd1_0520.pdb |
| SEQ ID NO: 2907 | HEEH_rd3_0009.pdb |
| SEQ ID NO: 2908 | HHH_rd1_0524.pdb |
| SEQ ID NO: 2909 | EEHEE_rd4_0697.pdb |
| SEQ ID NO: 2910 | EHEE_rd4_0214.pdb |
| SEQ ID NO: 2911 | HHH_rd1_0269.pdb |
| SEQ ID NO: 2912 | EHEE_rd4_0694.pdb |
| SEQ ID NO: 2913 | EEHEE_rd3_1189.pdb |
| SEQ ID NO: 2914 | EEHEE_rd4_0662.pdb |
| SEQ ID NO: 2915 | EHEE_rd2_0097.pdb |
| SEQ ID NO: 2916 | EHEE_rd3_0172.pdb |
| SEQ ID NO: 2917 | EHEE_rd2_0412.pdb |
| SEQ ID NO: 2918 | EEHEE_rd3_0668.pdb |
| SEQ ID NO: 2919 | HEEH_rd4_0565.pdb |
| SEQ ID NO: 2920 | EEHEE_rd4_0669.pdb |
| SEQ ID NO: 2921 | EHEE_rd4_0459.pdb |
| SEQ ID NO: 2922 | HEEH_rd2_0471.pdb |
| SEQ ID NO: 2923 | HHH_rd4_0530.pdb |
| SEQ ID NO: 2924 | EHEE_rd1_0536.pdb |
| SEQ ID NO: 2925 | EEHEE_rd4_0220.pdb |
| SEQ ID NO: 2926 | EEHEE_rd4_0642.pdb |
| SEQ ID NO: 2927 | EEHEE_rd4_0433.pdb |
| SEQ ID NO: 2928 | HHH_rd1_0803.pdb |
| SEQ ID NO: 2929 | HHH_rd3_0009.pdb |
| SEQ ID NO: 2930 | EHEE_rd4_0016.pdb |
| SEQ ID NO: 2931 | EEHEE_rd4_0090.pdb |
| SEQ ID NO: 2932 | EHEE_rd4_0751.pdb |
| SEQ ID NO: 2933 | HHH_rd1_0141.pdb |
| SEQ ID NO: 2934 | HHH_rd4_0839.pdb |
| SEQ ID NO: 2935 | EHEE_rd2_0600.pdb |
| SEQ ID NO: 2936 | HHH_rd2_0140.pdb |
| SEQ ID NO: 2937 | EHEE_rd4_0312.pdb |
| SEQ ID NO: 2938 | HHH_rd4_0652.pdb |
| SEQ ID NO: 2939 | EHEE_rd4_0759.pdb |
| SEQ ID NO: 2940 | EEHEE_rd3_0403.pdb |
| SEQ ID NO: 2941 | EEHEE_rd3_0355.pdb |
| SEQ ID NO: 2942 | HHH_rd4_0829.pdb |
| SEQ ID NO: 2943 | EEHEE_rd4_0773.pdb |
| SEQ ID NO: 2944 | HHH_rd1_0832.pdb |
| SEQ ID NO: 2945 | EHEE_rd3_0147.pdb |
| SEQ ID NO: 2946 | HHH_rd1_0013.pdb |
| SEQ ID NO: 2947 | HHH_rd4_0703.pdb |
| SEQ ID NO: 2948 | HHH_rd4_0677.pdb |
| SEQ ID NO: 2949 | EEHEE_rd3_1717.pdb |
| SEQ ID NO: 2950 | EEHEE_rd3_0273.pdb |
| SEQ ID NO: 2951 | EHEE_rd4_0390.pdb |
| SEQ ID NO: 2952 | EEHEE_rd4_0505.pdb |
| SEQ ID NO: 2953 | EHEE_rd4_0886.pdb |
| SEQ ID NO: 2954 | EEHEE_rd4_0406.pdb |
| SEQ ID NO: 2955 | HHH_rd4_0184.pdb |
| SEQ ID NO: 2956 | EHEE_rd3_0047.pdb |
| SEQ ID NO: 2957 | EEHEE_rd3_0101.pdb |
| SEQ ID NO: 2958 | HHH_rd2_0068.pdb |
| SEQ ID NO: 2959 | EHEE_rd4_0916.pdb |
| SEQ ID NO: 2960 | HEEH_rd3_0083.pdb |
| SEQ ID NO: 2961 | HHH_rd1_0039.pdb |
| SEQ ID NO: 2962 | HEEH_rd3_0049.pdb |
| SEQ ID NO: 2963 | EHEE_rd4_0061.pdb |
| SEQ ID NO: 2964 | HHH_rd4_0912.pdb |
| SEQ ID NO: 2965 | EHEE_rd4_0902.pdb |
| SEQ ID NO: 2966 | EEHEE_rd4_0372.pdb |
| SEQ ID NO: 2967 | EEHEE_rd2_0554.pdb |
| SEQ ID NO: 2968 | EHEE_rd4_0107.pdb |
| SEQ ID NO: 2969 | EEHEE_rd4_0503.pdb |
| SEQ ID NO: 2970 | EEHEE_rd3_1515.pdb |
| SEQ ID NO: 2971 | EHEE_rd4_0724.pdb |
| SEQ ID NO: 2972 | HHH_rd4_0140.pdb |
| SEQ ID NO: 2973 | EHEE_rd2_0696.pdb |
| SEQ ID NO: 2974 | EHEE_rd4_0803.pdb |
| SEQ ID NO: 2975 | EEHEE_rd3_0107.pdb |
| SEQ ID NO: 2976 | HEEH_rd3_0416.pdb |
| SEQ ID NO: 2977 | EHEE_rd4_0555.pdb |
| SEQ ID NO: 2978 | EEHEE_rd4_0906.pdb |
| SEQ ID NO: 2979 | EEHEE_rd4_0450.pdb |
| SEQ ID NO: 2980 | EEHEE_rd4_0438.pdb |
| SEQ ID NO: 2981 | EEHEE_rd4_0453.pdb |
| SEQ ID NO: 2982 | HEEH_rd4_0601.pdb |
| SEQ ID NO: 2983 | EEHEE_rd3_0402.pdb |
| SEQ ID NO: 2984 | EEHEE_rd4_0170.pdb |
| SEQ ID NO: 2985 | EEHEE_rd4_0727.pdb |
| SEQ ID NO: 2986 | EEHEE_rd4_0095.pdb |
| SEQ ID NO: 2987 | EHEE_rd4_0769.pdb |
| SEQ ID NO: 2988 | EEHEE_rd3_1213.pdb |
| SEQ ID NO: 2989 | EEHEE_rd3_1813.pdb |
| SEQ ID NO: 2990 | HHH_rd3_0070.pdb |
| SEQ ID NO: 2991 | HHH_rd4_0620.pdb |
| SEQ ID NO: 2992 | EEHEE_rd3_0339.pdb |
| SEQ ID NO: 2993 | EHEE_rd4_0180.pdb |
| SEQ ID NO: 2994 | HHH_rd4_0510.pdb |
| SEQ ID NO: 2995 | EEHEE_rd4_0396.pdb |
| SEQ ID NO: 2996 | EEHEE_rd3_0873.pdb |
| SEQ ID NO: 2997 | HHH_rd4_0030.pdb |
| SEQ ID NO: 2998 | EHEE_rd3_0181.pdb |
| SEQ ID NO: 2999 | HHH_rd4_0697.pdb |
| SEQ ID NO: 3000 | HHH_rd4_0922.pdb |
| SEQ ID NO: 3001 | EHEE_rd4_0038.pdb |
| SEQ ID NO: 3002 | HHH_rd1_0577.pdb |
| SEQ ID NO: 3003 | EHEE_rd4_0193.pdb |
| SEQ ID NO: 3004 | HHH_rd4_0513.pdb |
| SEQ ID NO: 3005 | HEEH_rd3_0373.pdb |
| SEQ ID NO: 3006 | EEHEE_rd4_0944.pdb |
| SEQ ID NO: 3007 | EEHEE_rd4_0228.pdb |
| SEQ ID NO: 3008 | HHH_rd3_0141.pdb |
| SEQ ID NO: 3009 | EEHEE_rd3_1345.pdb |
| SEQ ID NO: 3010 | EEHEE_rd4_0984.pdb |
| SEQ ID NO: 3011 | EEHEE_rd4_0404.pdb |
| SEQ ID NO: 3012 | EHEE_rd2_0198.pdb |
| SEQ ID NO: 3013 | EEHEE_rd4_0920.pdb |
| SEQ ID NO: 3014 | EHEE_rd2_0311.pdb |
| SEQ ID NO: 3015 | EEHEE_rd3_0077.pdb |
| SEQ ID NO: 3016 | HHH_rd1_0306.pdb |
| SEQ ID NO: 3017 | EHEE_rd1_0491.pdb |
| SEQ ID NO: 3018 | EEHEE_rd3_0241.pdb |
| SEQ ID NO: 3019 | HHH_rd1_0075.pdb |
| SEQ ID NO: 3020 | EEHEE_rd3_0008.pdb |
| SEQ ID NO: 3021 | EHEE_rd4_0373.pdb |
| SEQ ID NO: 3022 | HHH_rd4_0419.pdb |
| SEQ ID NO: 3023 | EHEE_rd4_0755.pdb |
| SEQ ID NO: 3024 | EHEE_rd4_0327.pdb |
| SEQ ID NO: 3025 | EEHEE_rd4_0247.pdb |
| SEQ ID NO: 3026 | EHEE_rd4_0779.pdb |
| SEQ ID NO: 3027 | EEHEE_rd3_1307.pdb |
| SEQ ID NO: 3028 | EEHEE_rd4_0286.pdb |
| SEQ ID NO: 3029 | HHH_rd1_0183.pdb |
| SEQ ID NO: 3030 | EHEE_rd4_0899.pdb |
| SEQ ID NO: 3031 | EEHEE_rd2_0913.pdb |
| SEQ ID NO: 3032 | HHH_rd2_0144.pdb |
| SEQ ID NO: 3033 | HHH_rd2_0174.pdb |
| SEQ ID NO: 3034 | EEHEE_rd4_0783.pdb |
| SEQ ID NO: 3035 | HHH_rd3_0099.pdb |
| SEQ ID NO: 3036 | EHEE_rd4_0798.pdb |
| SEQ ID NO: 3037 | EEHEE_rd3_0283.pdb |
| SEQ ID NO: 3038 | EHEE_rd3_0247.pdb |
| SEQ ID NO: 3039 | EHEE_rd4_0578.pdb |
| SEQ ID NO: 3040 | EEHEE_rd4_0964.pdb |
| SEQ ID NO: 3041 | EHEE_rd4_0907.pdb |
| SEQ ID NO: 3042 | HHH_rd4_0902.pdb |
| SEQ ID NO: 3043 | HHH_rd1_1818.pdb |
| SEQ ID NO: 3044 | EEHEE_rd4_0033.pdb |
| SEQ ID NO: 3045 | EEHEE_rd3_0740.pdb |
| SEQ ID NO: 3046 | HEEH_rd2_0086.pdb |
| SEQ ID NO: 3047 | EHEE_rd4_0078.pdb |
| SEQ ID NO: 3048 | HHH_rd3_0214.pdb |
| SEQ ID NO: 3049 | EEHEE_rd3_1092.pdb |
| SEQ ID NO: 3050 | EEHEE_rd3_0725.pdb |
| SEQ ID NO: 3051 | EEHEE_rd4_0689.pdb |
| SEQ ID NO: 3052 | EEHEE_rd4_0444.pdb |
| SEQ ID NO: 3053 | EHEE_rd4_0537.pdb |
| SEQ ID NO: 3054 | EHEE_rd4_0175.pdb |
| SEQ ID NO: 3055 | EEHEE_rd4_0128.pdb |
| SEQ ID NO: 3056 | EEHEE_rd3_1467.pdb |
| SEQ ID NO: 3057 | HEEH_rd4_0279.pdb |
| SEQ ID NO: 3058 | EEHEE_rd3_1361.pdb |
| SEQ ID NO: 3059 | HHH_rd4_0814.pdb |
| SEQ ID NO: 3060 | HEEH_rd4_0009.pdb |
| SEQ ID NO: 3061 | EHEE_rd2_0558.pdb |
| SEQ ID NO: 3062 | EHEE_rd4_0498.pdb |
| SEQ ID NO: 3063 | HEEH_rd4_0226.pdb |
| SEQ ID NO: 3064 | HHH_rd4_0906.pdb |
| SEQ ID NO: 3065 | HHH_rd4_0864.pdb |
| SEQ ID NO: 3066 | EEHEE_rd4_0677.pdb |
| SEQ ID NO: 3067 | HEEH_rd4_0323.pdb |
| SEQ ID NO: 3068 | HEEH_rd3_0436.pdb |
| SEQ ID NO: 3069 | HEEH_rd3_1294.pdb |
| SEQ ID NO: 3070 | EHEE_rd4_0620.pdb |
| SEQ ID NO: 3071 | EKEE_rd4_0404.pdb |
| SEQ ID NO: 3072 | EEHEE_rd3_0289.pdb |
| SEQ ID NO: 3073 | EHEE_rd4_0577.pdb |
| SEQ ID NO: 3074 | EEHEE_rd4_0703.pdb |
| SEQ ID NO: 3075 | EHEE_rd4_0452.pdb |
| SEQ ID NO: 3076 | HHH_rd4_0731.pdb |
| SEQ ID NO: 3077 | EEHEE_rd3_0775.pdb |
| SEQ ID NO: 3078 | HEEH_rd3_0747.pdb |
| SEQ ID NO: 3079 | EEHEE_rd4_0922.pdb |
| SEQ ID NO: 3080 | EEHEE_rd3_1701.pdb |
| SEQ ID NO: 3081 | HHH_rd1_0557.pdb |
| SEQ ID NO: 3082 | EEHEE_rd4_0120.pdb |
| SEQ ID NO: 3083 | HHH_rd3_0054.pdb |
| SEQ ID NO: 3084 | HEEH_rd3_0192.pdb |
| SEQ ID NO: 3085 | HHH_rd4_0594.pdb |
| SEQ ID NO: 3086 | EEHEE_rd3_1774.pdb |
| SEQ ID NO: 3087 | EEHEE_rd4_0435.pdb |
| SEQ ID NO: 3088 | HHH_rd2_0011.pdb |
| SEQ ID NO: 3089 | EHEE_rd4_0305.pdb |
| SEQ ID NO: 3090 | EEHEE_rd3_1099.pdb |
| SEQ ID NO: 3091 | EHEE_rd2_0808.pdb |
| SEQ ID NO: 3092 | EEHEE_rd3_1154.pdb |
| SEQ ID NO: 3093 | HHH_rd1_0516.pdb |
| SEQ ID NO: 3094 | EEHEE_rd4_0004.pdb |
| SEQ ID NO: 3095 | HEEH_rd3_0399.pdb |
| SEQ ID NO: 3096 | HHH_rd1_0749.pdb |
| SEQ ID NO: 3097 | EHEE_rd4_0013.pdb |
| SEQ ID NO: 3098 | EEHEE_rd3_1273.pdb |
| SEQ ID NO: 3099 | HEEH_rd3_0616.pdb |
| SEQ ID NO: 3100 | EHEE_rd4_0051.pdb |
| SEQ ID NO: 3101 | EHEE_rd4_0762.pdb |
| SEQ ID NO: 3102 | HHH_rd4_0773.pdb |
| SEQ ID NO: 3103 | EEHEE_rd3_1519.pdb |
| SEQ ID NO: 3104 | EHEE_rd4_0675.pdb |
| SEQ ID NO: 3105 | HHH_rd1_0511.pdb |
| SEQ ID NO: 3106 | HEEH_rd3_1217.pdb |
| SEQ ID NO: 3107 | HEEH_rd2_0032.pdb |
| SEQ ID NO: 3108 | HHH_rd4_0421.pdb |
| SEQ ID NO: 3109 | EHEE_rd2_1165.pdb |
| SEQ ID NO: 3110 | EHEE_rd4_0785.pdb |
| SEQ ID NO: 3111 | EEHEE_rd4_0715.pdb |
| SEQ ID NO: 3112 | EEHEE_rd4_0264.pdb |
| SEQ ID NO: 3113 | EHEE_rd4_0710.pdb |
| SEQ ID NO: 3114 | HHH_rd1_0277.pdb |
| SEQ ID NO: 3115 | HEEH_rd3_0301.pdb |
| SEQ ID NO: 3116 | EEHEE_rd3_0227.pdb |
| SEQ ID NO: 3117 | EHEE_rd4_0092.pdb |
| SEQ ID NO: 3118 | EEHEE_rd4_0790.pdb |
| SEQ ID NO: 3119 | EHEE_rd4_0940.pdb |
| SEQ ID NO: 3120 | EHEE_rd4_0387.pdb |
| SEQ ID NO: 3121 | EEHEE_rd3_1106.pdb |
| SEQ ID NO: 3122 | HHH_rd4_0622.pdb |
| SEQ ID NO: 3123 | EEHEE_rd4_0947.pdb |
| SEQ ID NO: 3124 | HEEH_rd3_0502.pdb |
| SEQ ID NO: 3125 | EHEE_rd4_0623.pdb |
| SEQ ID NO: 3126 | EEHEE_rd4_0528.pdb |
| SEQ ID NO: 3127 | EHEE_rd4_0455.pdb |
| SEQ ID NO: 3128 | EEHEE_rd3_0811.pdb |
| SEQ ID NO: 3129 | EHEE_rd4_0011.pdb |
| SEQ ID NO: 3130 | HHH_rd1_0555.pdb |
| SEQ ID NO: 3131 | EEHEE_rd3_1091.pdb |
| SEQ ID NO: 3132 | EEHEE_rd3_1255.pdb |
| SEQ ID NO: 3133 | EEHEE_rd4_0728.pdb |
| SEQ ID NO: 3134 | EEHEE_rd4_0966.pdb |
| SEQ ID NO: 3135 | HEEH_rd3_0567.pdb |
| SEQ ID NO: 3136 | EHEE_rd3_0001.pdb |
| SEQ ID NO: 3137 | EEHEE_rd3_0645.pdb |
| SEQ ID NO: 3138 | EEHEE_rd4_0113.pdb |
| SEQ ID NO: 3139 | HHH_rd4_0136.pdb |
| SEQ ID NO: 3140 | EHEE_rd4_0566.pdb |
| SEQ ID NO: 3141 | EHEE_rd2_0559.pdb |
| SEQ ID NO: 3142 | EEHEE_rd4_0816.pdb |
| SEQ ID NO: 3143 | EHEE_rd4_0687.pdb |
| SEQ ID NO: 3144 | EHEE_rd2_0797.pdb |
| SEQ ID NO: 3145 | EEHEE_rd3_1171.pdb |
| SEQ ID NO: 3146 | EHEE_rd4_0063.pdb |
| SEQ ID NO: 3147 | EHEE_rd2_0425.pdb |
| SEQ ID NO: 3148 | EHEE_rd4_0289.pdb |
| SEQ ID NO: 3149 | EHEE_rd4_0845.pdb |
| SEQ ID NO: 3150 | HEEH_rd2_0599.pdb |
| SEQ ID NO: 3151 | HHH_rd4_0408.pdb |
| SEQ ID NO: 3152 | EHEE_rd4_0141.pdb |
| SEQ ID NO: 3153 | EHEE_rd4_0799.pdb |
| SEQ ID NO: 3154 | EHH_rd4_0439.pdb |
| SEQ ID NO: 3155 | EEHEE_rd4_0667.pdb |
| SEQ ID NO: 3156 | EHEE_rd2_0152.pdb |
| SEQ ID NO: 3157 | EEHEE_rd4_0567.pdb |
| SEQ ID NO: 3158 | HHH_rd1_0669.pdb |
| SEQ ID NO: 3159 | HHH_rd4_0137.pdb |
| SEQ ID NO: 3160 | HHH_rd1_0702.pdb |
| SEQ ID NO: 3161 | HEEH_rd1_0741.pdb |
| SEQ ID NO: 3162 | HHH_rd4_0062.pdb |
| SEQ ID NO: 3163 | EHEE_rd1_0945.pdb |
| SEQ ID NO: 3164 | EHEE_rd4_0362.pdb |
| SEQ ID NO: 3165 | EHEE_rd2_0402.pdb |
| SEQ ID NO: 3166 | EEHEE_rd3_1272.pdb |
| SEQ ID NO: 3167 | EEHEE_rd4_0734.pdb |
| SEQ ID NO: 3168 | EEHEE_rd3_0587.pdb |
| SEQ ID NO: 3169 | EEHEE_rd3_0683.pdb |
| SEQ ID NO: 3170 | EHEE_rd2_0236.pdb |
| SEQ ID NO: 3171 | HHH_rd1_0046.pdb |
| SEQ ID NO: 3172 | HHH_rd1_0053.pdb |
| SEQ ID NO: 3173 | HHH_rd4_0905.pdb |
| SEQ ID NO: 3174 | HHH_rd4_0194.pdb |
| SEQ ID NO: 3175 | EEHEE_rd4_0974.pdb |
| SEQ ID NO: 3176 | HEEH_rd2_0465.pdb |
| SEQ ID NO: 3177 | EEHEE_rd3_1275.pdb |
| SEQ ID NO: 3178 | EEHEE_rd4_0488.pdb |
| SEQ ID NO: 3179 | HHH_rd4_0844.pdb |
| SEQ ID NO: 3180 | HHH_rd3_0115.pdb |
| SEQ ID NO: 3181 | EEHEE_rd3_0011.pdb |
| SEQ ID NO: 3182 | HHH_rd4_0280.pdb |
| SEQ ID NO: 3183 | EEHEE_rd3_1069.pdb |
| SEQ ID NO: 3184 | EHEE_rd4_0124.pdb |
| SEQ ID NO: 3185 | EHEE_rd3_0200.pdb |
| SEQ ID NO: 3186 | HHH_rd4_0223.pdb |
| SEQ ID NO: 3187 | EHEE_rd3_0114.pdb |
| SEQ ID NO: 3188 | EEHEE_rd4_0437.pdb |
| SEQ ID NO: 3189 | EEHEE_rd4_0671.pdb |
| SEQ ID NO: 3190 | HEEH_rd4_0566.pdb |
| SEQ ID NO: 3191 | EHEE_rd4_0929.pdb |
| SEQ ID NO: 3192 | EEHEE_rd3_1076.pdb |
| SEQ ID NO: 3193 | EEHEE_rd4_0103.pdb |
| SEQ ID NO: 3194 | HHH_rd1_0440.pdb |
| SEQ ID NO: 3195 | EEHEE_rd3_1322.pdb |
| SEQ ID NO: 3196 | EHEE_rd4_0355.pdb |
| SEQ ID NO: 3197 | HHH_rd1_0996.pdb |
| SEQ ID NO: 3198 | EEHEE_rd3_1401.pdb |
| SEQ ID NO: 3199 | EEHEE_rd4_0351.pdb |
| SEQ ID NO: 3200 | EEHEE_rd3_1622.pdb |
| SEQ ID NO: 3201 | EHEE_rd2_0075.pdb |
| SEQ ID NO: 3202 | EEHEE_rd3_1647.pdb |
| SEQ ID NO: 3203 | HHH_rd4_0596.pdb |
| SEQ ID NO: 3204 | EEHEE_rd3_0748.pdb |
| SEQ ID NO: 3205 | HHH_rd4_0330.pdb |
| SEQ ID NO: 3206 | EEHEE_rd4_0346.pdb |
| SEQ ID NO: 3207 | HHH_rd3_0192.pdb |
| SEQ ID NO: 3208 | HHH_rd1_0304.pdb |
| SEQ ID NO: 3209 | HHH_rd3_0102.pdb |
| SEQ ID NO: 3210 | EHEE_rd3_0122.pdb |
| SEQ ID NO: 3211 | EHEE_rd4_0421.pdb |
| SEQ ID NO: 3212 | HHH_rd1_0911.pdb |
| SEQ ID NO: 3213 | EEHEE_rd4_0139.pdb |
| SEQ ID NO: 3214 | HEEH_rd3_0139.pdb |
| SEQ ID NO: 3215 | EEHEE_rd3_0010.pdb |
| SEQ ID NO: 3216 | HHH_rd4_0503.pdb |
| SEQ ID NO: 3217 | EEHEE_rd3_1587.pdb |
| SEQ ID NO: 3218 | EEHEE_rd4_0527.pdb |
| SEQ ID NO: 3219 | EEHEE_rd4_0914.pdb |
| SEQ ID NO: 3220 | EHEE_rd4_0313.pdb |
| SEQ ID NO: 3221 | EEHEE_rd3_0942.pdb |
| SEQ ID NO: 3222 | HEEH_rd4_0097.pdb |
| SEQ ID NO: 3223 | EEHEE_rd4_0534.pdb |
| SEQ ID NO: 3224 | EHEE_rd2_1019.pdb |
| SEQ ID NO: 3225 | EEHEE_rd4_0854.pdb |
| SEQ ID NO: 3226 | HHH_rd1_0711.pdb |
| SEQ ID NO: 3227 | HHH_rd4_0788.pdb |
| SEQ ID NO: 3228 | EEHEE_rd3_0315.pdb |
| SEQ ID NO: 3229 | EEHEE_rd3_0932.pdb |
| SEQ ID NO: 3230 | EEHEE_rd4_0496.pdb |
| SEQ ID NO: 3231 | EHEE_rd2_0229.pdb |
| SEQ ID NO: 3232 | EHEE_rd4_0229.pdb |
| SEQ ID NO: 3233 | EHEE_rd4_0286.pdb |
| SEQ ID NO: 3234 | HHH_rd4_0807.pdb |
| SEQ ID NO: 3235 | EEHEE_rd4_0808.pdb |
| SEQ ID NO: 3236 | EEHEE_rd3_0298.pdb |
| SEQ ID NO: 3237 | EEHEE_rd3_0243.pdb |
| SEQ ID NO: 3238 | EHEE_rd4_0052.pdb |
| SEQ ID NO: 3239 | EEHEE_rd4_0269.pdb |
| SEQ ID NO: 3240 | EEHEE_rd4_0441.pdb |
| SEQ ID NO: 3241 | EEHEE_rd4_0300.pdb |
| SEQ ID NO: 3242 | HHH_rd4_0546.pdb |
| SEQ ID NO: 3243 | EEHEE_rd4_0770.pdb |
| SEQ ID NO: 3244 | HHH_rd3_0171.pdb |
| SEQ ID NO: 3245 | HHH_rd4_0307.pdb |
| SEQ ID NO: 3246 | EEHEE_rd3_0706.pdb |
| SEQ ID NO: 3247 | EEHEE_rd3_1000.pdb |
| SEQ ID NO: 3248 | EHEE_rd4_05153.pdb |
| SEQ ID NO: 3249 | EHEE_rd4_0767.pdb |
| SEQ ID NO: 3250 | EHEE_rd4_0209.pdb |
| SEQ ID NO: 3251 | EEHEE_rd4_0342.pdb |
| SEQ ID NO: 3252 | EEHEE_rd3_1208.pdb |
| SEQ ID NO: 3253 | EEHEE_rd4_0235.pdb |
| SEQ ID NO: 3254 | EEHEE_rd4_0745.pdb |
| SEQ ID NO: 3255 | HHH_rd4_0369.pdb |
| SEQ ID NO: 3256 | HHH_rd4_0959.pdb |
| SEQ ID NO: 3257 | HHH_rd4_0182.pdb |
| SEQ ID NO: 3258 | EEHEE_rd3_0945.pdb |
| SEQ ID NO: 3259 | HHH_rd1_0715.pdb |
| SEQ ID NO: 3260 | EEHEE_rd3_0948.pdb |
| SEQ ID NO: 3261 | HHH_rd1_0052.pdb |
| SEQ ID NO: 3262 | EHEE_rd4_0245.pdb |
| SEQ ID NO: 3263 | HEEH_rd3_0111.pdb |
| SEQ ID NO: 3264 | HHH_rd1_0473.pdb |
| SEQ ID NO: 3265 | HHH_rd3_0196.pdb |
| SEQ ID NO: 3266 | HEEH_rd3_1413.pdb |
| SEQ ID NO: 3267 | EEHEE_rd4_0699.pdb |
| SEQ ID NO: 3268 | HHH_rd4_0096.pdb |
| SEQ ID NO: 3269 | EEHEE_rd4_0730.pdb |
| SEQ ID NO: 3270 | HEEH_rd3_0088.pdb |
| SEQ ID NO: 3271 | EHEE_rd4_0417.pdb |
| SEQ ID NO: 3272 | EEHEE_rd4_0291.pdb |
| SEQ ID NO: 3273 | EEHEE_rd4_0681.pdb |
| SEQ ID NO: 3274 | EEHEE_rd3_1117.pdb |
| SEQ ID NO: 3275 | EEHEE_rd4_0370.pdb |
| SEQ ID NO: 3276 | EEHEE_rd4_0408.pdb |
| SEQ ID NO: 3277 | EEHEE_rd3_0499.pdb |
| SEQ ID NO: 3278 | EEHEE_rd4_0832.pdb |
| SEQ ID NO: 3279 | EHEE_rd4_0156.pdb |
| SEQ ID NO: 3280 | HEEH_rd2_0646.pdb |
| SEQ ID NO: 3281 | EHEE_rd4_0885.pdb |
| SEQ ID NO: 3282 | EEHEE_rd4_0733.pdb |
| SEQ ID NO: 3283 | EEHEE_rd1_0699.pdb |
| SEQ ID NO: 3284 | EEHEE_rd3_1365.pdb |
| SEQ ID NO: 3285 | EHEE_rd4_0105.pdb |
| SEQ ID NO: 3286 | EEHEE_rd4_0571.pdb |
| SEQ ID NO: 3287 | EHEE_rd4_0103.pdb |
| SEQ ID NO: 3288 | EEHEE_rd4_0884.pdb |
| SEQ ID NO: 3289 | EHEE_rd4_0483.pdb |
| SEQ ID NO: 3290 | EEHEE_rd3_0066.pdb |
| SEQ ID NO: 3291 | EHEE_rd4_0891.pdb |
| SEQ ID NO: 3292 | EHEE_rd4_0501.pdb |
| SEQ ID NO: 3293 | EHEE_rd4_0138.pdb |
| SEQ ID NO: 3294 | HHH_rd3_0082.pdb |
| SEQ ID NO: 3295 | HHH_rd4_0336.pdb |
| SEQ ID NO: 3296 | EEHEE_rd3_0944.pdb |
| SEQ ID NO: 3297 | EHEE_rd3_0073.pdb |
| SEQ ID NO: 3298 | HHH_rd1_0908.pdb |
| SEQ ID NO: 3299 | EEHEE_rd3_0025.pdb |
| SEQ ID NO: 3300 | EHEE_rd4_0511.pdb |
| SEQ ID NO: 3301 | HHH_rd4_0851.pdb |
| SEQ ID NO: 3302 | EEHEE_rd4_0794.pdb |
| SEQ ID NO: 3303 | HHH_rd1_0942.pdb |
| SEQ ID NO: 3304 | HHH_rd2_0146.pdb |
| SEQ ID NO: 3305 | HHH_rd3_0168.pdb |
| SEQ ID NO: 3306 | HHH_rd4_0995.pdb |
| SEQ ID NO: 3307 | EEHEE_rd4_0474.pdb |
| SEQ ID NO: 3308 | HHH_rd3_0067.pdb |
| SEQ ID NO: 3309 | EHEE_rd2_0022.pdb |
| SEQ ID NO: 3310 | HEEH_rd3_0080.pdb |
| SEQ ID NO: 3311 | EEHEE_rd4_0279.pdb |
| SEQ ID NO: 3312 | HEEH_rd3_0890.pdb |
| SEQ ID NO: 3313 | HEEH_rd3_0068.pdb |
| SEQ ID NO: 3314 | HEEH_rd3_0224.pdb |
| SEQ ID NO: 3315 | HHH_rd4_0700.pdb |
| SEQ ID NO: 3316 | HEEH_rd4_0162.pdb |
| SEQ ID NO: 3317 | EHEE_rd3_0021.pdb |
| SEQ ID NO: 3318 | HEEH_rd3_0154.pdb |
| SEQ ID NO: 3319 | EEHEE_rd3_0046.pdb |
| SEQ ID NO: 3320 | HEEH_rd3_0387.pdb |
| SEQ ID NO: 3321 | HHH_rd4_0410.pdb |
| SEQ ID NO: 3322 | EEHEE_rd3_1696.pdb |
| SEQ ID NO: 3323 | HEEH_rd2_0128.pdb |
| SEQ ID NO: 3324 | EHEE_rd4_0199.pdb |
| SEQ ID NO: 3325 | EHEE_rd1_0654.pdb |
| SEQ ID NO: 3326 | HEEH_rd3_0886.pdb |
| SEQ ID NO: 3327 | HEEH_rd3_1362.pdb |
| SEQ ID NO: 3328 | HHH_rd4_0496.pdb |
| SEQ ID NO: 3329 | EEHEE_rd4_0108.pdb |
| SEQ ID NO: 3330 | EEHEE_rd4_0254.pdb |
| SEQ ID NO: 3331 | EEHEE_rd4_0317.pdb |
| SEQ ID NO: 3332 | EHEE_rd4_0661.pdb |
| SEQ ID NO: 3333 | EHEE_rd1_0295.pdb |
| SEQ ID NO: 3334 | EEHEE_rd4_0915.pdb |
| SEQ ID NO: 3335 | HHH_rd4_0985.pdb |
| SEQ ID NO: 3336 | HHH_rd3_0187.pdb |
| SEQ ID NO: 3337 | EEHEE_rd3_1087.pdb |
| SEQ ID NO: 3338 | HEEH_rd4_0076.pdb |
| SEQ ID NO: 3339 | EEHEE_rd3_1198.pdb |
| SEQ ID NO: 3340 | EEHEE_rd4_0844.pdb |
| SEQ ID NO: 3341 | EHEE_rd4_0591.pdb |
| SEQ ID NO: 3342 | EEHEE_rd4_0985.pdb |
| SEQ ID NO: 3343 | HHH_rd3_0066.pdb |
| SEQ ID NO: 3344 | HHH_rd4_0924.pdb |
| SEQ ID NO: 3345 | HHH_rd3_0232.pdb |
| SEQ ID NO: 3346 | EHEE_rd2_0104.pdb |
| SEQ ID NO: 3347 | HHH_rd1_0730.pdb |
| SEQ ID NO: 3348 | EEHEE_rd4_0587.pdb |
| SEQ ID NO: 3349 | EEHEE_rd2_1166.pdb |
| SEQ ID NO: 3350 | EEHEE_rd4_0687.pdb |
| SEQ ID NO: 3351 | HHH_rd4_0783.pdb |
| SEQ ID NO: 3352 | HHH_rd1_0442.pdb |
| SEQ ID NO: 3353 | HHH_rd1_0512.pdb |
| SEQ ID NO: 3354 | EHEE_rd4_0469.pdb |
| SEQ ID NO: 3355 | HHH_rd4_0724.pdb |
| SEQ ID NO: 3356 | HHH_rd3_0025.pdb |
| SEQ ID NO: 3357 | EHEE_rd2_0069.pdb |
| SEQ ID NO: 3358 | EEHEE_rd3_1229.pdb |
| SEQ ID NO: 3359 | HHH_rd1_0494.pdb |
| SEQ ID NO: 3360 | HEEH_rd3_0484.pdb |
| SEQ ID NO: 3361 | EEHEE_rd3_0720.pdb |
| SEQ ID NO: 3362 | HHH_rd3_0217.pdb |
| SEQ ID NO: 3363 | HEEH_rd4_0175.pdb |
| SEQ ID NO: 3364 | EEHEE_rd3_0466.pdb |
| SEQ ID NO: 3365 | HHH_rd4_0430.pdb |
| SEQ ID NO: 3366 | EEHEE_rd3_0800.pdb |
| SEQ ID NO: 3367 | EEHEE_rd3_1252.pdb |
| SEQ ID NO: 3368 | HHH_rd1_0212.pdb |
| SEQ ID NO: 3369 | EEHEE_rd3_0252.pdb |
| SEQ ID NO: 3370 | EEHEE_rd3_1532.pdb |
| SEQ ID NO: 3371 | EHEE_rd4_0738.pdb |
| SEQ ID NO: 3372 | EEHEE_rd4_0987.pdb |
| SEQ ID NO: 3373 | EEHEE_rd4_0880.pdb |
| SEQ ID NO: 3374 | HEEH_rd3_1405.pdb |
| SEQ ID NO: 3375 | EEHEE_rd3_0388.pdb |
| SEQ ID NO: 3376 | EEHEE_rd3_0367.pdb |
| SEQ ID NO: 3377 | EEHEE_rd4_0389.pdb |
| SEQ ID NO: 3378 | EHEE_rd2_0827.pdb |
| SEQ ID NO: 3379 | EEHEE_rd3_1775.pdb |
| SEQ ID NO: 3380 | HHH_rd1_0796.pdb |
| SEQ ID NO: 3381 | HHH_rd1_0319.pdb |
| SEQ ID NO: 3382 | HEEH_rd3_1605.pdb |
| SEQ ID NO: 3383 | EEHEE_rd3_1462.pdb |
| SEQ ID NO: 3384 | EHEE_rd4_0593.pdb |
| SEQ ID NO: 3385 | EEHEE_rd4_0603.pdb |
| SEQ ID NO: 3386 | EEHEE_rd3_0369.pdb |
| SEQ ID NO: 3387 | HHH_rd1_0545.pdb |
| SEQ ID NO: 3388 | HEEH_rd2_0301.pdb |
| SEQ ID NO: 3389 | EHEE_rd2_1284.pdb |
| SEQ ID NO: 3390 | EHEE_rd4_0169.pdb |
| SEQ ID NO: 3391 | EEHEE_rd4_0942.pdb |
| SEQ ID NO: 3392 | EHEE_rd4_0957.pdb |
| SEQ ID NO: 3393 | HEEH_rd2_0192.pdb |
| SEQ ID NO: 3394 | EEHEE_rd4_0386.pdb |
| SEQ ID NO: 3395 | HEEH_rd3_0084.pdb |
| SEQ ID NO: 3396 | HEEH_rd2_0485.pdb |
| SEQ ID NO: 3397 | HEEH_rd3_0854.pdb |
| SEQ ID NO: 3398 | HHH_rd1_0515.pdb |
| SEQ ID NO: 3399 | EEHEE_rd4_0999.pdb |
| SEQ ID NO: 3400 | HEEH_rd3_1153.pdb |
| SEQ ID NO: 3401 | HHH_rd1_0771.pdb |
| SEQ ID NO: 3402 | EHEE_rd4_0665.pdb |
| SEQ ID NO: 3403 | HHH_rd3_0143.pdb |
| SEQ ID NO: 3404 | HHH_rd1_0276.pdb |
| SEQ ID NO: 3405 | EEHEE_rd4_0936.pdb |
| SEQ ID NO: 3406 | HEEH_rd4_0503.pdb |
| SEQ ID NO: 3407 | EHEE_rd3_0098.pdb |
| SEQ ID NO: 3408 | HHH_rd1_0845.pdb |
| SEQ ID NO: 3409 | EHEE_rd3_0177.pdb |
| SEQ ID NO: 3410 | EEHEE_rd3_0411.pdb |
| SEQ ID NO: 3411 | EHEE_rd2_1132.pdb |
| SEQ ID NO: 3412 | HHH_rd4_0976.pdb |
| SEQ ID NO: 3413 | EEHEE_rd3_1825.pdb |
| SEQ ID NO: 3414 | EHEE_rd2_1103.pdb |
| SEQ ID NO: 3415 | HEEH_rd3_1614.pdb |
| SEQ ID NO: 3416 | EHEE_rd4_0863.pdb |
| SEQ ID NO: 3417 | HHH_rd1_0055.pdb |
| SEQ ID NO: 3418 | EHEE_rd4_0766.pdb |
| SEQ ID NO: 3419 | EEHEE_rd3_0422.pdb |
| SEQ ID NO: 3420 | HHH_rd3_0158.pdb |
| SEQ ID NO: 3421 | EEHEE_rd4_0722.pdb |
| SEQ ID NO: 3422 | HHH_rd1_0027.pdb |
| SEQ ID NO: 3423 | EEHEE_rd4_0849.pdb |
| SEQ ID NO: 3424 | EEHEE_rd4_0556.pdb |
| SEQ ID NO: 3425 | EEHEE_rd4_0246.pdb |
| SEQ ID NO: 3426 | EHEE_rd4_0277.pdb |
| SEQ ID NO: 3427 | HHH_rd1_0600.pdb |
| SEQ ID NO: 3428 | EEHEE_rd2_0853.pdb |
| SEQ ID NO: 3429 | EEHEE_rd3_1718.pdb |
| SEQ ID NO: 3430 | HEEH_rd3_0573.pdb |
| SEQ ID NO: 3431 | EEHEE_rd3_1496.pdb |
| SEQ ID NO: 3432 | EHEE_rd4_0619.pdb |
| SEQ ID NO: 3433 | HHH_rd1_0445.pdb |
| SEQ ID NO: 3434 | EEHEE_rd3_0691.pdb |
| SEQ ID NO: 3435 | EHEE_rd4_0989.pdb |
| SEQ ID NO: 3436 | EEHEE_rd4_0993.pdb |
| SEQ ID NO: 3437 | EEHEE_rd3_0559.pdb |
| SEQ ID NO: 3438 | EHEE_rd3_0246.pdb |
| SEQ ID NO: 3439 | HEEH_rd3_0148.pdb |
| SEQ ID NO: 3440 | HEEH_rd3_1274.pdb |
| SEQ ID NO: 3441 | EEHEE_rd4_0561.pdb |
| SEQ ID NO: 3442 | HEEH_rd4_0249.pdb |
| SEQ ID NO: 3443 | HEEH_rd4_0962.pdb |
| SEQ ID NO: 3444 | EEHEE_rd4_0292.pdb |
| SEQ ID NO: 3445 | EHEE_rd4_0482.pdb |
| SEQ ID NO: 3446 | HHH_rd1_0147.pdb |
| SEQ ID NO: 3447 | HHH_rd1_0146.pdb |
| SEQ ID NO: 3448 | EHEE_rd2_0150.pdb |
| SEQ ID NO: 3449 | EHEE_rd2_0015.pdb |
| SEQ ID NO: 3450 | EHEE_rd4_0830.pdb |
| SEQ ID NO: 3451 | HEEH_rd3_0179.pdb |
| SEQ ID NO: 3452 | EEHEE_rd3_1663.pdb |
| SEQ ID NO: 3453 | EHEE_rd4_0526.pdb |
| SEQ ID NO: 3454 | EHEE_rd2_1088.pdb |
| SEQ ID NO: 3455 | HEEH_rd2_0432.pdb |
| SEQ ID NO: 3456 | HEEH_rd3_1798.pdb |
| SEQ ID NO: 3457 | HHH_rd1_0596.pdb |
| SEQ ID NO: 3458 | EHEE_rd2_0998.pdb |
| SEQ ID NO: 3459 | EEHEE_rd3_1089.pdb |
| SEQ ID NO: 3460 | HEEH_rd4_0035.pdb |
| SEQ ID NO: 3461 | EEHEE_rd4_0501.pdb |
| SEQ ID NO: 3462 | EHEE_rd2_1087.pdb |
| SEQ ID NO: 3463 | EHEE_rd4_0106.pdb |
| SEQ ID NO: 3464 | EEHEE_rd3_0904.pdb |
| SEQ ID NO: 3465 | EHEE_rd2_1066.pdb |
| SEQ ID NO: 3466 | EHEE_rd4_0986.pdb |
| SEQ ID NO: 3467 | EHEE_rd3_0116.pdb |
| SEQ ID NO: 3468 | EHEE_rd4_0316.pdb |
| SEQ ID NO: 3469 | EEHEE_rd4_0440.pdb |
| SEQ ID NO: 3470 | EHEE_rd4_0443.pdb |
| SEQ ID NO: 3471 | EEHEE_rd4_0889.pdb |
| SEQ ID NO: 3472 | HEEH_rd1_0727.pdb |
| SEQ ID NO: 3473 | EHEE_rd4_0218.pdb |
| SEQ ID NO: 3474 | EHEE_rd4_0389.pdb |
| SEQ ID NO: 3475 | EHEE_rd3_0049.pdb |
| SEQ ID NO: 3476 | EEHEE_rd4_0825.pdb |
| SEQ ID NO: 3477 | EHEE_rd1_0307.pdb |
| SEQ ID NO: 3478 | HHH_rd1_0016.pdb |
| SEQ ID NO: 3479 | EEHEE_rd3_1247.pdb |
| SEQ ID NO: 3480 | EEHEE_rd3_0201.pdb |
| SEQ ID NO: 3481 | HHH_rd1_0405.pdb |
| SEQ ID NO: 3482 | HHH_rd1_0132.pdb |
| SEQ ID NO: 3483 | EEHEE_rd4_0732.pdb |
| SEQ ID NO: 3484 | EHEE_rd4_0301.pdb |
| SEQ ID NO: 3485 | EHEE_rd4_0409.pdb |
| SEQ ID NO: 3486 | EEHEE_rd3_1786.pdb |
| SEQ ID NO: 3487 | HHH_rd1_0228.pdb |
| SEQ ID NO: 3488 | EEHEE_rd4_0443.pdb |
| SEQ ID NO: 3489 | EEHEE_rd3_1704.pdb |
| SEQ ID NO: 3490 | EEHEE_rd4_0708.pdb |
| SEQ ID NO: 3491 | HHH_rd1_0199.pdb |
| SEQ ID NO: 3492 | HEEH_rd3_1358.pdb |
| SEQ ID NO: 3493 | HEEH_rd4_0364.pdb |
| SEQ ID NO: 3494 | HHH_rd1_0467.pdb |
| SEQ ID NO: 3495 | EHEE_rd4_0143.pdb |
| SEQ ID NO: 3496 | EHEE_rd4_0984.pdb |
| SEQ ID NO: 3497 | HHH_rd4_0749.pdb |
| SEQ ID NO: 3498 | EEHEE_rd4_0853.pdb |
| SEQ ID NO: 3499 | HHH_rd1_0679.pdb |
| SEQ ID NO: 3500 | EEHEE_rd4_0457.pdb |
| SEQ ID NO: 3501 | EHEE_rd4_0269.pdb |
| SEQ ID NO: 3502 | HHH_rd1_0490.pdb |
| SEQ ID NO: 3503 | HEEH_rd2_0726.pdb |
| SEQ ID NO: 3504 | HEEH_rd3_0874.pdb |
| SEQ ID NO: 3505 | EEHEE_rd4_0536.pdb |
| SEQ ID NO: 3506 | HEEH_rd3_0677.pdb |
| SEQ ID NO: 3507 | EEHEE_rd4_0673.pdb |
| SEQ ID NO: 3508 | HEEH_rd3_1466.pdb |
| SEQ ID NO: 3509 | EEHEE_rd4_0419.pdb |
| SEQ ID NO: 3510 | EEHEE_rd3_1356.pdb |
| SEQ ID NO: 3511 | HEEH_rd4_0898.pdb |
| SEQ ID NO: 3512 | HHH_rd1_0683.pdb |
| SEQ ID NO: 3513 | HHH_rd2_0099.pdb |
| SEQ ID NO: 3514 | EHEE_rd1_0025.pdb |
| SEQ ID NO: 3515 | HHH_rd4_0718.pdb |
| SEQ ID NO: 3516 | HHH_rd1_0130.pdb |
| SEQ ID NO: 3517 | EHEE_rd1_0926.pdb |
| SEQ ID NO: 3518 | HEEH_rd4_0146.pdb |
| SEQ ID NO: 3519 | EEHEE_rd4_0787.pdb |
| SEQ ID NO: 3520 | HHH_rd1_0129.pdb |
| SEQ ID NO: 3521 | EEHEE_rd3_0064.pdb |
| SEQ ID NO: 3522 | EEHEE_rd4_0919.pdb |
| SEQ ID NO: 3523 | EHEE_rd4_0854.pdb |
| SEQ ID NO: 3524 | EEHEE_rd4_0201.pdb |
| SEQ ID NO: 3525 | EEHEE_rd3_1521.pdb |
| SEQ ID NO: 3526 | EEHEE_rd3_0398.pdb |
| SEQ ID NO: 3527 | EEHEE_rd3_0979.pdb |
| SEQ ID NO: 3528 | EEHEE_rd4_0041.pdb |
| SEQ ID NO: 3529 | HHH_rd4_0555.pdb |
| SEQ ID NO: 3530 | HHH_rd4_0825.pdb |
| SEQ ID NO: 3531 | EHEE_rd2_0571.pdb |
| SEQ ID NO: 3532 | EHEE_rd4_0120.pdb |
| SEQ ID NO: 3533 | HHH_rd1_0077.pdb |
| SEQ ID NO: 3534 | EHEE_rd4_0627.pdb |
| SEQ ID NO: 3535 | EEHEE_rd4_0643.pdb |
| SEQ ID NO: 3536 | EEHEE_rd4_0654.pdb |
| SEQ ID NO: 3537 | HEEH_rd3_0701.pdb |
| SEQ ID NO: 3538 | HEEH_rd4_0400.pdb |
| SEQ ID NO: 3539 | EEHEE_rd3_0739.pdb |
| SEQ ID NO: 3540 | EEHEE_rd3_0554.pdb |
| SEQ ID NO: 3541 | EEHEE_rd4_0423.pdb |
| SEQ ID NO: 3542 | EHEE_rd2_0546.pdb |
| SEQ ID NO: 3543 | HEEH_rd3_0362.pdb |
| SEQ ID NO: 3544 | EEHEE_rd3_1659.pdb |
| SEQ ID NO: 3545 | EEHEE_rd3_0535.pdb |
| SEQ ID NO: 3546 | HHH_rd1_0352.pdb |
| SEQ ID NO: 3547 | EEHEE_rd3_1044.pdb |
| SEQ ID NO: 3548 | EHEE_rd3_0048.pdb |
| SEQ ID NO: 3549 | HEEH_rd3_1323.pdb |
| SEQ ID NO: 3550 | EEHEE_rd3_0092.pdb |
| SEQ ID NO: 3551 | EHEE_rd1_0357.pdb |
| SEQ ID NO: 3552 | EEHEE_rd4_0909.pdb |
| SEQ ID NO: 3553 | EEHEE_rd3_1535.pdb |
| SEQ ID NO: 3554 | EEhEE_rd2_0753.pdb |
| SEQ ID NO: 3555 | EEHEE_rd4_0815.pdb |
| SEQ ID NO: 3556 | EEHEE_rd3_0915.pdb |
| SEQ ID NO: 3557 | EEHEE_rd3_0200.pdb |
| SEQ ID NO: 3558 | HEEH_rd2_0244.pdb |
| SEQ ID NO: 3559 | HEEH_rd4_0709.pdb |
| SEQ ID NO: 3560 | EHEE_rd4_0870.pdb |
| SEQ ID NO: 3561 | EEHEE_rd4_0744.pdb |
| SEQ ID NO: 3562 | EHEE_rd2_1292.pdb |
| SEQ ID NO: 3563 | HHH_rd1_0628.pdb |
| SEQ ID NO: 3564 | HEEH_rd3_1528.pdb |
| SEQ ID NO: 3565 | EEHEE_rd4_0755.pdb |
| SEQ ID NO: 3566 | EHEE_rd4_0807.pdb |
| SEQ ID NO: 3567 | HHH_rd4_0678.pdb |
| SEQ ID NO: 3568 | EHEE_rd3_0132.pdb |
| SEQ ID NO: 3569 | EEHEE_rd4_0885.pdb |
| SEQ ID NO: 3570 | EEHEE_rd4_0262.pdb |
| SEQ ID NO: 3571 | HHH_rd4_0726.pdb |
| SEQ ID NO: 3572 | EEHEE_rd4_0837.pdb |
| SEQ ID NO: 3573 | HHH_rd2_0092.pdb |
| SEQ ID NO: 3574 | EEHEE_rd3_1425.pdb |
| SEQ ID NO: 3575 | HEEH_rd4_0349.pdb |
| SEQ ID NO: 3576 | EEHEE_rd3_0627.pdb |
| SEQ ID NO: 3577 | HHH_rd4_0761.pdb |
| SEQ ID NO: 3578 | HEEH_rd3_0764.pdb |
| SEQ ID NO: 3579 | HEEH_rd4_0276.pdb |
| SEQ ID NO: 3580 | EHEE_rd4_0695.pdb |
| SEQ ID NO: 3581 | EEHEE_rd3_0534.pdb |
| SEQ ID NO: 3582 | HHH_rd2_0027.pdb |
| SEQ ID NO: 3583 | EHEE_rd2_0493.pdb |
| SEQ ID NO: 3584 | HHH_rd1_0312.pdb |
| SEQ ID NO: 3585 | HHH_rd1_0042.pdb |
| SEQ ID NO: 3586 | HHH_rd3_0077.pdb |
| SEQ ID NO: 3587 | HHH_rd1_0462.pdb |
| SEQ ID NO: 3588 | EEHEE_rd3_0386.pdb |
| SEQ ID NO: 3589 | EEHEE_rd3_0926.pdb |
| SEQ ID NO: 3590 | HEEH_rd4_0011.pdb |
| SEQ ID NO: 3591 | EHEE_rd2_0543.pdb |
| SEQ ID NO: 3592 | EEHEE_rd4_0823.pdb |
| SEQ ID NO: 3593 | EHEE_rd2_0817.pdb |
| SEQ ID NO: 3594 | EEHEE_rd4_0461.pdb |
| SEQ ID NO: 3595 | EEHEE_rd3_0886.pdb |
| SEQ ID NO: 3596 | HHH_rd4_0845.pdb |
| SEQ ID NO: 3597 | EEHEE_rd4_0564.pdb |
| SEQ ID NO: 3598 | EEHEE_rd3_1688.pdb |
| SEQ ID NO: 3599 | EHEE_rd4_0497.pdb |
| SEQ ID NO: 3600 | EEHEE_rd4_0925.pdb |
| SEQ ID NO: 3601 | EHEE_rd2_0252.pdb |
| SEQ ID NO: 3602 | HHH_rd4_0473.pdb |
| SEQ ID NO: 3603 | EHEE_rd2_0541.pdb |
| SEQ ID NO: 3604 | EHEE_rd4_0744.pdb |
| SEQ ID NO: 3605 | EEHEE_rd2_0965.pdb |
| SEQ ID NO: 3606 | EEHEE_rd4_0554.pdb |
| SEQ ID NO: 3607 | EEHEE_rd3_1047.pdb |
| SEQ ID NO: 3608 | HHH_rd1_0211.pdb |
| SEQ ID NO: 3609 | HHH_rd1_0128.pdb |
| SEQ ID NO: 3610 | HHH_rd4_6214.pdb |
| SEQ ID NO: 3611 | EEHEE_rd2_0925.pdb |
| SEQ ID NO: 3612 | HHH_rd1_0919.pdb |
| SEQ ID NO: 3613 | EEHEE_rd4_0305.pdb |
| SEQ ID NO: 3614 | HEEH_rd2_0682.pdb |
| SEQ ID NO: 3615 | EEHEE_rd4_0330.pdb |
| SEQ ID NO: 3616 | EEHEE_rd2_0959.pdb |
| SEQ ID NO: 3617 | EHEE_rd3_0131.pdb |
| SEQ ID NO: 3618 | EEHEE_rd3_1357.pdb |
| SEQ ID NO: 3619 | EEHEE_rd3_0340.pdb |
| SEQ ID NO: 3620 | HHH_rd1_0189.pdb |
| SEQ ID NO: 3621 | EHEE_rd4_0199.pdb |
| SEQ ID NO: 3622 | EEHEE_rd4_0930.pdb |
| SEQ ID NO: 3623 | HHH_rd1_0999.pdb |
| SEQ ID NO: 3624 | EEHEE_rd3_0019.pdb |
| SEQ ID NO: 3625 | EHEE_rd4_0520.pdb |
| SEQ ID NO: 3626 | EHEE_rd2_0802.pdb |
| SEQ ID NO: 3627 | EEHEE_rd4_0192.pdb |
| SEQ ID NO: 3628 | EHEE_rd4_0032.pdb |
| SEQ ID NO: 3629 | EHEE_rd4_0802.pdb |
| SEQ ID NO: 3630 | EEHEE_rd3_0649.pdb |
| SEQ ID NO: 3631 | HEEH_rd3_0830.pdb |
| SEQ ID NO: 3632 | EEHEE_rd4_0402.pdb |
| SEQ ID NO: 3633 | EHEE_rd1_0326.pdb |
| SEQ ID NO: 3634 | HHH_rd1_0157.pdb |
| SEQ ID NO: 3635 | EHEE_rd4_0178.pdb |
| SEQ ID NO: 3636 | EEHEE_rd3_1029.pdb |
| SEQ ID NO: 3637 | EEHEE_rd4_0574.pdb |
| SEQ ID NO: 3638 | HHH_rd1_0986.pdb |
| SEQ ID NO: 3639 | EEHEE_rd4_0916.pdb |
| SEQ ID NO: 3640 | EHEE_rd4_0721.pdb |
| SEQ ID NO: 3641 | EEHEE_rd4_0047.pdb |
| SEQ ID NO: 3642 | HEEH_rd4_0197.pdb |
| SEQ ID NO: 3643 | EEHEE_rd4_0851.pdb |
| SEQ ID NO: 3644 | EEHEE_rd3_0973.pdb |
| SEQ ID NO: 3645 | HEEH_rd4_0303.pdb |
| SEQ ID NO: 3646 | HEEH_rd4_0461.pdb |
| SEQ ID NO: 3647 | EEHEE_rd4_0872.pdb |
| SEQ ID NO: 3648 | HHH_rd1_0069.pdb |
| SEQ ID NO: 3649 | EEHEE_rd4_0569.pdb |
| SEQ ID NO: 3650 | EEHEE_rd3_0589.pdb |
| SEQ ID NO: 3651 | EHEE_rd2_0723.pdb |
| SEQ ID NO: 3652 | HHH_rd4_0897.pdb |
| SEQ ID NO: 3653 | EHEE_rd4_0888.pdb |
| SEQ ID NO: 3654 | HEEH_rd4_0596.pdb |
| SEQ ID NO: 3655 | EHEE_rd3_0076.pdb |
| SEQ ID NO: 3656 | HHH_rd1_0665.pdb |
| SEQ ID NO: 3657 | HHH_rd1_p448.pdb |
| SEQ ID NO: 3658 | EHEE_rd2_1133.pdb |
| SEQ ID NO: 3659 | EEHEE_rd4_0604.pdb |
| SEQ ID NO: 3660 | EHEE_rd4_0615.pdb |
| SEQ ID NO: 3661 | EEHEE_rd4_0962.pdb |
| SEQ ID NO: 3662 | EEHEE_rd1_0123.pdb |
| SEQ ID NO: 3663 | EEHEE_rd3_1448.pdb |
| SEQ ID NO: 3664 | EHEE_rd1_0257.pdb |
| SEQ ID NO: 3665 | EEHEE_rd3_0498.pdb |
| SEQ ID NO: 3666 | EEHEE_rd4_0760.pdb |
| SEQ ID NO: 3667 | HEEH_rd3_0997.pdb |
| SEQ ID NO: 3668 | HEEH_rd1_0959.pdb |
| SEQ ID NO: 3669 | EEHEE_rd3_0851.pdb |
| SEQ ID NO: 3670 | HEEH_rd4_0401.pdb |
| SEQ ID NO: 3671 | HEEH_rd4_0316.pdb |
| SEQ ID NO: 3672 | HEEH_rd2_1070.pdb |
| SEQ ID NO: 3673 | EHEE_rd4_0376.pdb |
| SEQ ID NO: 3674 | EHEE_rd2_p325.pdb |
| SEQ ID NO: 3675 | HEEH_rd3_0074.pdb |
| SEQ ID NO: 3676 | EHEE_rd2_1152.pdb |
| SEQ ID NO: 3677 | EEHEE_rd4_0248.pdb |
| SEQ ID NO: 3678 | EHEE_rd2_0751.pdb |
| SEQ ID NO: 3679 | EEHEE_rd4_0625.pdb |
| SEQ ID NO: 3680 | EHEE_rd4_0257.pdb |
| SEQ ID NO: 3681 | HEEH_rd4_0010.pdb |
| SEQ ID NO: 3682 | HEEH_rd4_0607.pdb |
| SEQ ID NO: 3683 | EHEE_rd4_0635.pdb |
| SEQ ID NO: 3684 | EHEE_rd4_0100.pdb |
| SEQ ID NO: 3685 | EHEE_rd4_0131.pdb |
| SEQ ID NO: 3686 | HHH_rd4_0297.pdb |
| SEQ ID NO: 3687 | HEEH_rd4_0081.pdb |
| SEQ ID NO: 3688 | EEHEE_rd4_0958.pdb |
| SEQ ID NO: 3689 | EHEE_rd4_0002.pdb |
| SEQ ID NO: 3690 | HEEH_rd4_0222.pdb |
| SEQ ID NO: 3691 | EEHEE_rd3_0050.pdb |
| SEQ ID NO: 3692 | EEHEE_rd3_1212.pdb |
| SEQ ID NO: 3693 | HEEH_rd2_1111.pdb |
| SEQ ID NO: 3694 | HEEH_rd3_0756.pdb |
| SEQ ID NO: 3695 | EEHEE_rd3_0484.pdb |
| SEQ ID NO: 3696 | EEHEE_rd3_0347.pdb |
| SEQ ID NO: 3697 | EHEE_rd2_0950.pdb |
| SEQ ID NO: 3698 | EEHEE_rd3_0196.pdb |
| SEQ ID NO: 3699 | EHEE_rd2_0410.pdb |
| SEQ ID NO: 3700 | EEHEE_rd4_0666.pdb |
| SEQ ID NO: 3701 | EEHEE_rd4_0584.pdb |
| SEQ ID NO: 3702 | EEHEE_rd3_1011.pdb |
| SEQ ID NO: 3703 | EEHEE_rd3_0527.pdb |
| SEQ ID NO: 3704 | EEHEE_rd4_0834.pdb |
| SEQ ID NO: 3705 | EEHEE_rd4_0494.pdb |
| SEQ ID NO: 3706 | HEEH_rd2_1223.pdb |
| SEQ ID NO: 3707 | EEHEE_rd2_0242.pdb |
| SEQ ID NO: 3708 | HHH_rd2_0164.pdb |
| SEQ ID NO: 3709 | HHH_rd2_0168.pdb |
| SEQ ID NO: 3710 | EHEE_rd2_0757.pdb |
| SEQ ID NO: 3711 | EEHEE_rd3_1233.pdb |
| SEQ ID NO: 3712 | EHEE_rd2_1173.pdb |
| SEQ ID NO: 3713 | HEEH_rd3_1034.pdb |
| SEQ ID NO: 3714 | EEHEE_rd3_0497.pdb |
| SEQ ID NO: 3715 | HEEH_rd4_0342.pdb |
| SEQ ID NO: 3716 | EHEE_rd4_0568.pdb |
| SEQ ID NO: 3717 | EEHEE_rd3_0920.pdb |
| SEQ ID NO: 3718 | EEHEE_rd3_0854.pdb |
| SEQ ID NO: 3719 | EEHEE_rd4_0707.pdb |
| SEQ ID NO: 3720 | HEEH_rd3_0188.pdb |
| SEQ ID NO: 3721 | HHH_rd1_0430.pdb |
| SEQ ID NO: 3722 | EEHEE_rd3_1325.pdb |
| SEQ ID NO: 3723 | EEHEE_rd3_1172.pdb |
| SEQ ID NO: 3724 | HEEH_rd4_0680.pdb |
| SEQ ID NO: 3725 | EHEE_rd4_0137.pdb |
| SEQ ID NO: 3726 | EEHEE_rd4_0874.pdb |
| SEQ ID NO: 3727 | EEHEE_rd3_1192.pdb |
| SEQ ID NO: 3728 | EEHEE_rd3_1181.pdb |
| SEQ ID NO: 3729 | EEHEE_rd4_0481.pdb |
| SEQ ID NO: 3730 | HHH_rd1_0387.pdb |
| SEQ ID NO: 3731 | HHH_rd1_0232.pdb |
| SEQ ID NO: 3732 | EEHEE_rd3_0754.pdb |
| SEQ ID NO: 3733 | EHEE_rd3_0166.pdb |
| SEQ ID NO: 3734 | EHEE_rd4_0274.pdb |
| SEQ ID NO: 3735 | EHEE_rd3_0227.pdb |
| SEQ ID NO: 3736 | EEHEE_rd3_1583.pdb |
| SEQ ID NO: 3737 | EHEE_rd4_0946.pdb |
| SEQ ID NO: 3738 | HHH_rd1_0781.pdb |
| SEQ ID NO: 3739 | EHEE_rd4_0659.pdb |
| SEQ ID NO: 3740 | EEHEE_rd3_0264.pdb |
| SEQ ID NO: 3741 | EEHEE_rd3_1380.pdb |
| SEQ ID NO: 3742 | EHEE_rd4_0928.pdb |
| SEQ ID NO: 3743 | EEHEE_rd3_0719.pdb |
| SEQ ID NO: 3744 | EEHEE_rd4_0928.pdb |
| SEQ ID NO: 3745 | EHEE_rd4_0632.pdb |
| SEQ ID NO: 3746 | EEHEE_rd4_0628.pdb |
| SEQ ID NO: 3747 | EHEE_rd4_0249.pdb |
| SEQ ID NO: 3748 | HHH_rd3_0039.pdb |
| SEQ ID NO: 3749 | HEEH_rd1_0620.pdb |
| SEQ ID NO: 3750 | EHEE_rd4_0395.pdb |
| SEQ ID NO: 3751 | HEEH_rd3_0061.pdb |
| SEQ ID NO: 3752 | EHEE_rd3_0243.pdb |
| SEQ ID NO: 3753 | HHH_rd1_0891.pdb |
| SEQ ID NO: 3754 | EEHEE_rd4_0911.pdb |
| SEQ ID NO: 3755 | HHH_rd1_0856.pdb |
| SEQ ID NO: 3756 | EEHEE_rd4_0509.pdb |
| SEQ ID NO: 3757 | EEHEE_rd4_0746.pdb |
| SEQ ID NO: 3758 | EEHEE_rd3_0415.pdb |
| SEQ ID NO: 3759 | EEHEE_rd4_0720.pdb |
| SEQ ID NO: 3760 | EEHEE_rd3_0391.pdb |
| SEQ ID NO: 3761 | EHEE_rd4_0542.pdb |
| SEQ ID NO: 3762 | EHEE_rd4_0309.pdb |
| SEQ ID NO: 3763 | EHEE_rd2_1251.pdb |
| SEQ ID NO: 3764 | EHEE_rd4_0745.pdb |
| SEQ ID NO: 3765 | EEHEE_rd3_0597.pdb |
| SEQ ID NO: 3766 | EEHEE_rd3_0029.pdb |
| SEQ ID NO: 3767 | EEHEE_rd4_0335.pdb |
| SEQ ID NO: 3768 | EEHEE_rd4_0619.pdb |
| SEQ ID NO: 3769 | HHH_rd1_0109.pdb |
| SEQ ID NO: 3770 | EEHEE_rd3_0065.pdb |
| SEQ ID NO: 3771 | HHH_rd4_0909.pdb |
| SEQ ID NO: 3772 | HHH_rd1_0563.pdb |
| SEQ ID NO: 3773 | EEHEE_rd3_0185.pdb |
| SEQ ID NO: 3774 | EEHEE_rd3_1157.pdb |
| SEQ ID NO: 3775 | EEHEE_rd4_0622.pdb |
| SEQ ID NO: 3776 | HEEH_rd2_1299.pdb |
| SEQ ID NO: 3777 | HEEH_rd3_0286.pdb |
| SEQ ID NO: 3778 | HHH_rd1_0226.pdb |
| SEQ ID NO: 3779 | EHEE_rd4_0804.pdb |
| SEQ ID NO: 3780 | HHH_rd4_0499.pdb |
| SEQ ID NO: 3781 | EHEE_rd3_0007.pdb |
| SEQ ID NO: 3782 | EEHEE_rd4_0473.pdb |
| SEQ ID NO: 3783 | EEHEE_rd3_0993.pdb |
| SEQ ID NO: 3784 | HHH_rd2_0147.pdb |
| SEQ ID NO: 3785 | EEHEE_rd3_0936.pdb |
| SEQ ID NO: 3786 | EEHEE_rd3_0779.pdb |
| SEQ ID NO: 3787 | HHH_rd4_0545.pdb |
| SEQ ID NO: 3788 | HHH_rd4_0483.pdb |
| SEQ ID NO: 3789 | HHH_rd3_0220.pdb |
| SEQ ID NO: 3790 | EHEE_rd4_0624.pdb |
| SEQ ID NO: 3791 | EEHEE_rd4_0385.pdb |
| SEQ ID NO: 3792 | EHEE_rd4_0606.pdb |
| SEQ ID NO: 3793 | EEHEE_rd4_0588.pdb |
| SEQ ID NO: 3794 | EEHEE_rd4_0949.pdb |
| SEQ ID NO: 3795 | EEHEE_rd3_0845.pdb |
| SEQ ID NO: 3796 | HEEH_rd2_0604.pdb |
| SEQ ID NO: 3797 | EEHEE_rd3_1721.pdb |
| SEQ ID NO: 3798 | HEEH_rd4_0065.pdb |
| SEQ ID NO: 3799 | HEEH_rd4_0060.pdb |
| SEQ ID NO: 3800 | HHH_rd1_0964.pdb |
| SEQ ID NO: 3801 | EEHEE_rd3_0384.pdb |
| SEQ ID NO: 3802 | HEEH_rd3_0666.pdb |
| SEQ ID NO: 3803 | HEEH_rd3_0462.pdb |
| SEQ ID NO: 3804 | HEEH_rd3_0035.pdb |
| SEQ ID NO: 3805 | EEHEE_rd4_0546.pdb |
| SEQ ID NO: 3806 | HEEH_rd3_0617.pdb |
| SEQ ID NO: 3807 | EEHEE_rd4_0600.pdb |
| SEQ ID NO: 3808 | EHEE_rd4_0933.pdb |
| SEQ ID NO: 3809 | EHEE_rd4_0641.pdb |
| SEQ ID NO: 3810 | EEHEE_rd4_0171.pdb |
| SEQ ID NO: 3811 | HHH_rd1_0116.pdb |
| SEQ ID NO: 3812 | HHH_rd1_0134.pdb |
| SEQ ID NO: 3813 | EHEE_rd4_0184.pdb |
| SEQ ID NO: 3814 | EHEE_rd2_1175.pdb |
| SEQ ID NO: 3815 | EEHEE_rd4_0530.pdb |
| SEQ ID NO: 3816 | EEHEE_rd3_0791.pdb |
| SEQ ID NO: 3817 | EHEE_rd4_0258.pdb |
| SEQ ID NO: 3818 | EHEE_rd4_0963.pdb |
| SEQ ID NO: 3819 | EEHEE_rd3_1283.pdb |
| SEQ ID NO: 3820 | HHH_rd1_0163.pdb |
| SEQ ID NO: 3821 | HHH_rd1_0977.pdb |
| SEQ ID NO: 3822 | EHEE_rd2_0388.pdb |
| SEQ ID NO: 3823 | HHH_rd4_0258.pdb |
| SEQ ID NO: 3824 | HHH_rd3_0149.pdb |
| SEQ ID NO: 3825 | EEHEE_rd4_0035.pdb |
| SEQ ID NO: 3826 | HEEH_rd3_1762.pdb |
| SEQ ID NO: 3827 | HHH_rd1_0831.pdb |
| SEQ ID NO: 3828 | EHEE_rd1_0742.pdb |
| SEQ ID NO: 3829 | EHEE_rd3_0060.pdb |
| SEQ ID NO: 3830 | EEHEE_rd4_0946.pdb |
| SEQ ID NO: 3831 | EHEE_rd2_0854.pdb |
| SEQ ID NO: 3832 | EEHEE_rd3_0917.pdb |
| SEQ ID NO: 3833 | EHEE_rd2_0371.pdb |
| SEQ ID NO: 3834 | EHEE_rd4_0676.pdb |
| SEQ ID NO: 3835 | EHEE_rd4_0736.pdb |
| SEQ ID NO: 3836 | HEEH_rd4_0363.pdb |
| SEQ ID NO: 3837 | EEHEE_rd4_0900.pdb |
| SEQ ID NO: 3838 | HEEH_rd4_0361.pdb |
| SEQ ID NO: 3839 | EHEE_rd2_0811.pdb |
| SEQ ID NO: 3840 | HHH_rd4_0591.pdb |
| SEQ ID NO: 3841 | EEHEE_rd4_0634.pdb |
| SEQ ID NO: 3842 | EHEE_rd3_0155.pdb |
| SEQ ID NO: 3843 | EEHEE_rd3_0488.pdb |
| SEQ ID NO: 3844 | HEEH_rd3_0405.pdb |
| SEQ ID NO: 3845 | EEHEE_rd3_1127.pdb |
| SEQ ID NO: 3846 | EEHEE_rd4_0940.pdb |
| SEQ ID NO: 3847 | EHEE_rd4_0704.pdb |
| SEQ ID NO: 3848 | EHEE_rd4_0926.pdb |
| SEQ ID NO: 3849 | EEHEE_rd4_0480.pdb |
| SEQ ID NO: 3850 | EEHEE_rd4_0768.pdb |
| SEQ ID NO: 3851 | HHH_rd1_0156.pdb |
| SEQ ID NO: 3852 | EEHEE_rd4_0926.pdb |
| SEQ ID NO: 3853 | EHEE_rd4_0903.pdb |
| SEQ ID NO: 3854 | HEEH_rd4_0003.pdb |
| SEQ ID NO: 3855 | HEEH_rd4_0568.pdb |
| SEQ ID NO: 3856 | EEHEE_rd4_0726.pdb |
| SEQ ID NO: 3857 | HEEH_rd3_0546.pdb |
| SEQ ID NO: 3858 | HHH_rd1_0513.pdb |
| SEQ ID NO: 3859 | HEEH_rd4_0435.pdb |
| SEQ ID NO: 3860 | HEEH_rd3_0528.pdb |
| SEQ ID NO: 3861 | HEEH_rd1_0169.pdb |
| SEQ ID NO: 3862 | EHEE_rd4_0800.pdb |
| SEQ ID NO: 3863 | HEEH_rd3_0167.pdb |
| SEQ ID NO: 3864 | EEHEE_rd3_0667.pdb |
| SEQ ID NO: 3865 | HHH_rd1_0553.pdb |
| SEQ ID NO: 3866 | HHH_rd1_0253.pdb |
| SEQ ID NO: 3867 | EHEE_rd2_0385.pdb |
| SEQ ID NO: 3868 | EHEE_rd4_0118.pdb |
| SEQ ID NO: 3869 | EEHEE_rd3_0815.pdb |
| SEQ ID NO: 3870 | HEEH_rd3_0678.pdb |
| SEQ ID NO: 3871 | HHH_rd3_0222.pdb |
| SEQ ID NO: 3872 | EHEE_rd4_0386.pdb |
| SEQ ID NO: 3873 | HHH_rd1_0394.pdb |
| SEQ ID NO: 3874 | EEHEE_rd2_1168.pdb |
| SEQ ID NO: 3875 | EHEE_rd4_0393.pdb |
| SEQ ID NO: 3876 | EEHEE_rd4_0859.pdb |
| SEQ ID NO: 3877 | HEEH_rd4_0121.pdb |
| SEQ ID NO: 3878 | EHEE_rd4_0034.pdb |
| SEQ ID NO: 3879 | HHH_rd2_0167.pdb |
| SEQ ID NO: 3880 | HEEH_rd2_0293.pdb |
| SEQ ID NO: 3881 | HEEH_rd3_1220.pdb |
| SEQ ID NO: 3882 | EEHEE_rd3_0808.pdb |
| SEQ ID NO: 3883 | HEEH_rd4_0887.pdb |
| SEQ ID NO: 3884 | EEHEE_rd2_0036.pdb |
| SEQ ID NO: 3885 | EEHEE_rd1_0294.pdb |
| SEQ ID NO: 3886 | EEHEE_rd3_0177.pdb |
| SEQ ID NO: 3887 | HHH_rd1_0659.pdb |
| SEQ ID NO: 3888 | HHH_rd2_0105.pdb |
| SEQ ID NO: 3889 | EHEE_rd4_0832.pdb |
| SEQ ID NO: 3890 | EHEE_rd4_0522.pdb |
| SEQ ID NO: 3891 | EHEE_rd2_1162.pdb |
| SEQ ID NO: 3892 | EHEE_rd4_0983.pdb |
| SEQ ID NO: 3893 | EEHEE_rd3_1305.pdb |
| SEQ ID NO: 3894 | EEHEE_rd3_1524.pdb |
| SEQ ID NO: 3895 | EHEE_rd4_0733.pdb |
| SEQ ID NO: 3896 | EEHEE_rd4_0933.pdb |
| SEQ ID NO: 3897 | EEHEE_rd2_0030.pdb |
| SEQ ID NO: 3898 | HEEH_rd1_0644.pdb |
| SEQ ID NO: 3899 | EEHEE_rd3_1173.pdb |
| SEQ ID NO: 3900 | EHEE_rd4_0565.pdb |
| SEQ ID NO: 3901 | EEHEE_rd3_0877.pdb |
| SEQ ID NO: 3902 | EEHEE_rd3_1593.pdb |
| SEQ ID NO: 3903 | EHEE_rd4_0846.pdb |
| SEQ ID NO: 3904 | HEEH_rd3_1395.pdb |
| SEQ ID NO: 3905 | EEHEE_rd3_1312.pdb |
| SEQ ID NO: 3906 | EEHEE_rd3_1020.pdb |
| SEQ ID NO: 3907 | EEHEE_rd3_0974.pdb |
| SEQ ID NO: 3908 | EEHEE_rd4_0651.pdb |
| SEQ ID NO: 3909 | EEHEE_rd3_0698.pdb |
| SEQ ID NO: 3910 | EEHEE_rd4_0952.pdb |
| SEQ ID NO: 3911 | HEEH_rd2_0344.pdb |
| SEQ ID NO: 3912 | EEHEE_rd3_0999.pdb |
| SEQ ID NO: 3913 | HEEH_rd3_1732.pdb |
| SEQ ID NO: 3914 | EEHEE_rd3_1624.pdb |
| SEQ ID NO: 3915 | HHH_rd1_0091.pdb |
| SEQ ID NO: 3916 | EEHEE_rd4_0253.pdb |
| SEQ ID NO: 3917 | HHH_rd2_0008.pdb |
| SEQ ID NO: 3918 | EHEE_rd1_0195.pdb |
| SEQ ID NO: 3919 | HEEH_rd4_0556.pdb |
| SEQ ID NO: 3920 | HHH_rd1_0180.pdb |
| SEQ ID NO: 3921 | HHH_rd1_0825.pdb |
| SEQ ID NO: 3922 | EEHEE_rd4_0692.pdb |
| SEQ ID NO: 3923 | EEHEE_rd4_0166.pdb |
| SEQ ID NO: 3924 | EHEE_rd3_0221.pdb |
| SEQ ID NO: 3925 | HHH_rd1_0649.pdb |
| SEQ ID NO: 3926 | EEHEE_rd2_0607.pdb |
| SEQ ID NO: 3927 | HEEH_rd3_0042.pdb |
| SEQ ID NO: 3928 | EEHEE_rd4_0606.pdb |
| SEQ ID NO: 3929 | HEEH_rd1_0964.pdb |
| SEQ ID NO: 3930 | EEHEE_rd3_1811.pdb |
| SEQ ID NO: 3931 | EHEE_rd4_0364.pdb |
| SEQ ID NO: 3932 | EEHEE_rd3_0323.pdb |
| SEQ ID NO: 3933 | EEHEE_rd3_0307.pdb |
| SEQ ID NO: 3934 | EEHEE_rd3_1699.pdb |
| SEQ ID NO: 3935 | HHH_rd1_0081.pdb |
| SEQ ID NO: 3936 | EHEE_rd4_0128.pdb |
| SEQ ID NO: 3937 | HEEH_rd1_0782.pdb |
| SEQ ID NO: 3938 | EEHEE_rd4_0812.pdb |
| SEQ ID NO: 3939 | EHEE_rd2_0275.pdb |
| SEQ ID NO: 3940 | EEHEE_rd3_0377.pdb |
| SEQ ID NO: 3941 | EEHEE_rd4_0234.pdb |
| SEQ ID NO: 3942 | EHEE_rd1_0475.pdb |
| SEQ ID NO: 3943 | EHEE_rd4_0399.pdb |
| SEQ ID NO: 3944 | EEHEE_rd4_0637.pdb |
| SEQ ID NO: 3945 | HEEH_rd3_1172.pdb |
| SEQ ID NO: 3946 | EHEE_rd4_0108.pdb |
| SEQ ID NO: 3947 | EEHEE_rd3_0647.pdb |
| SEQ ID NO: 3948 | HHH_rd3_0229.pdb |
| SEQ ID NO: 3949 | EEHEE_rd3_1567.pdb |
| SEQ ID NO: 3950 | HHH_rd1_0437.pdb |
| SEQ ID NO: 3951 | HEEH_rd3_0704.pdb |
| SEQ ID NO: 3952 | EHEE_rd4_0997.pdb |
| SEQ ID NO: 3953 | EEHEE_rd3_0410.pdb |
| SEQ ID NO: 3954 | EEHEE_rd3_1529.pdb |
| SEQ ID NO: 3955 | EHEE_rd4_0197.pdb |
| SEQ ID NO: 3956 | EHEE_rd4_0219.pdb |
| SEQ ID NO: 3957 | HEEH_rd2_0852.pdb |
| SEQ ID NO: 3958 | HEEH_rd1_0625.pdb |
| SEQ ID NO: 3959 | EHEE_rd1_0381.pdb |
| SEQ ID NO: 3960 | EHEE_rd4_0582.pdb |
| SEQ ID NO: 3961 | EEHEE_rd3_1463.pdb |
| SEQ ID NO: 3962 | EEHEE_rd3_1159.pdb |
| SEQ ID NO: 3963 | HEEH_rd2_0478.pdb |
| SEQ ID NO: 3964 | EHEE_rd4_0776.pdb |
| SEQ ID NO: 3965 | HEEH_rd3_1456.pdb |
| SEQ ID NO: 3966 | EHEE_rd2_0864.pdb |
| SEQ ID NO: 3967 | EEHEE_rd3_1166.pdb |
| SEQ ID NO: 3968 | EEHEE_rd4_0828.pdb |
| SEQ ID NO: 3969 | HEEH_rd4_0056.pdb |
| SEQ ID NO: 3970 | EHEE_rd2_0196.pdb |
| SEQ ID NO: 3971 | HEEH_rd4_0491.pdb |
| SEQ ID NO: 3972 | EHEE_rd4_0212.pdb |
| SEQ ID NO: 3973 | HEEH_rd4_0294.pdb |
| SEQ ID NO: 3974 | HEEH_rd4_0814.pdb |
| SEQ ID NO: 3975 | EEHEE_rd2_0900.pdb |
| SEQ ID NO: 3976 | EEHEE_rd3_0306.pdb |
| SEQ ID NO: 3977 | EEHEE_rd4_0401.pdb |
| SEQ ID NO: 3978 | HEEH_rd3_1651.pdb |
| SEQ ID NO: 3979 | EEHEE_rd4_0656.pdb |
| SEQ ID NO: 3980 | EEHEE_rd3_0015.pdb |
| SEQ ID NO: 3981 | EEHEE_rd3_1204.pdb |
| SEQ ID NO: 3982 | HEEH_rd4_0664.pdb |
| SEQ ID NO: 3983 | EEHEE_rd3_0275.pdb |
| SEQ ID NO: 3984 | HEEH_rd3_1309.pdb |
| SEQ ID NO: 3985 | EHEE_rd3_0160.pdb |
| SEQ ID NO: 3986 | EEHEE_rd4_0867.pdb |
| SEQ ID NO: 3987 | EEHEE_rd4_0757.pdb |
| SEQ ID NO: 3988 | EHEE_rd4_0519.pdb |
| SEQ ID NO: 3989 | EHEE_rd4_0334.pdb |
| SEQ ID NO: 3990 | EEHEE_rd4_0767.pdb |
| SEQ ID NO: 3991 | EEHEE_rd4_0575.pdb |
| SEQ ID NO: 3992 | HHH_rd4_0983.pdb |
| SEQ ID NO: 3993 | HEEH_rd3_1155.pdb |
| SEQ ID NO: 3994 | HHH_rd4_0961.pdb |
| SEQ ID NO: 3995 | HHH_rd1_0717.pdb |
| SEQ ID NO: 3996 | EEHEE_rd4_0245.pdb |
| SEQ ID NO: 3997 | HHH_rd3_0153.pdb |
| SEQ ID NO: 3998 | EHEE_rd3_0080.pdb |
| SEQ ID NO: 3999 | EHEE_rd4_0400.pdb |
| SEQ ID NO: 4000 | EEHEE_rd3_0638.pdb |

### REFERENCES

1. K. A. Dill, Dominant forces in protein folding. Biochemistry. 29, 7133-7155 (1990).
2. A. D. Robertson, K. P. Murphy, Protein Structure and the Energetics of Protein Stability. Chem. Rev. 97, 1251-1268 (1997).
3. C. Nick Pace, J. Martin Scholtz, G. R. Grimsley, Forces stabilizing proteins. FEBS Lett. 588, 2177-2184 (2014).
4. H. Gelman, M. Gruebele, Fast protein folding kinetics. Q. Rev. Biophys. 47, 95-142 (2014).
5. X. Jiang, J. Kowalski, J. W. Kelly, Increasing protein stability using a rational approach combining sequence homology and structural alignment: Stabilizing the WW domain. Protein Sci. 10, 1454-1465 (2001).
6. M. Jager et al., Structure-function-folding relationship in a WW domain. Proceedings of the National Academy of Sciences. 103, 10648-10653 (2006).
7. S. Xiao, Y. Bi, B. Shan, D. P. Raleigh, Analysis of core packing in a cooperatively folded miniature protein: the ultrafast folding villin headpiece helical subdomain. Biochemistry. 48, 4607-4616 (2009).
8. H. Neuweiler et al., The folding mechanism of BBL: Plasticity of transition-state structure observed within an ultrafast folding protein family. J. Mol. Biol. 390, 1060-1073 (2009).
9. C. N. Pace et al., Contribution of hydrophobic interactions to protein stability. J. Mol. Bol. 408, 514-528 (2011).
10. C. L. Araya et al., A fundamental protein property, thermodynamic stability, revealed solely from large-scale measurements of protein function. Proc. Natl. Acad. Sci. U.S.A. 109, 16858-16863 (2012).
11. S. Xiao et al., Rational modification of protein stability by targeting surface sites leads to complicated results. Proc. Natl. Acad. Sci. U.S.A. 110, 11337-11342 (2013).
12. C. N. Pace et al., Contribution of hydrogen bonds to protein stability. Protein Sci. 23, 652-661 (2014).
13. K. Lindorff-Larsen, S. Piana, R. O. Dror, D. E. Shaw, How Fast-Folding Proteins Fold. Science. 334, 517-520 (2011).
14. S. Piana, K. Lindorff-Larsen, D. E. Shaw, Protein folding kinetics and thermodynamics from atomistic simulation. Proc. Natl. Acad. Sci. U.S.A. 109, 17845-17850 (2012).
15. H. Nguyen, J. Maier, H. Huang, V. Perrone, C. Simmerling, Folding simulations for proteins with diverse topologies are accessible in days with a physics-based force field and implicit solvent. J. Am. Chem. Soc. 136, 13959-13962 (2014).
16. P.-S. Huang, S. E. Boyken, D. Baker, The coming of age of de novo protein design. Nature. 537, 320-327 (2016).
17. C. A. Rohl, C. E. M. Strauss, K. M. S. Misura, D. Baker, Protein structure prediction using Rosetta. Methods Enzymol. 383, 66-93 (2004).
18. T. J. Magliery, Protein stability: computation, sequence statistics, and new experimental methods. Curr. Opin. Struct. Biol. 33, 161-168 (2015).
19. H. Park et al., Simultaneous Optimization of Biomolecular Energy Functions on Features from Small Molecules and Macromolecules. J. Chem. Theory Comput. 12, 6201-6212 (2016).
20. B. 1. Dahiyat, S. L. Mayo, De novo protein design: fully automated sequence selection. Science. 278, 82-87 (1997).
21. H. Liang et al., De Novo Design of a βαβ Motif. Angew. Chem. Int. Ed. 48, 3301-3303 (2009).
22. S. Kosuri, G. M. Church, Large-scale de novo DNA synthesis: technologies and applications. Nat. Methods. 11, 499-507 (2014).
23. E. T. Boder, K. D. Wittrup, Yeast surface display for screening combinatorial polypeptide libraries. Nat. Biotechnol, 15, 553-557 (1997).
24. C. Park, S. Marqusee, Pulse proteolysis: a simple method for quantitative determination of protein stability and ligand binding. Nat. Methods. 2, 207-212 (2005).
25. C. Park, S. Zhou, J. Gilmore, S. Marqusee, Energetics-based protein profiling on a proteomic scale: identification of proteins resistant to proteolysis. J. Mol. Biol. 368, 1426-1437 (2007).
26. V. Sieber, A. Plückthun, F. X. Schmid, Selecting proteins with improved stability by a phage-based method. Nat. Biotechnol. 16, 955-960 (1998).
27. M. D. Finucane, M. Tuna, J. H. Lees, D. N. Woolfson, Core-directed protein design. I. An experimental method for selecting stable proteins from combinatorial libraries. Biochemistry. 38, 11604-11612 (1999).
28. M. Jager, M. Dendle, J. W. Kelly, Sequence determinants of thermodynamic stability in a WW domain—an all-beta-sheet protein. Protein Sci. 18, 1806-1813 (2009).
29. G. Bhardwaj et al., Accurate de novo design of hyperstable constrained peptides. Nature. 538, 329-335 (2016).
30. N. Koga et al., Principles for designing ideal protein structures. Nature. 491, 222-227 (2012).
31. S. Kamtekar, J. Schiffer, H. Xiong, J. Babik, M. Hecht, Protein design by binary patterning of polar and nonpolar amino acids. Science. 262, 1680-1685 (1993).
32. A. R. Davidson, R. T. Sauer, Folded proteins occur frequently in libraries of random amino acid sequences. Proc. Natl. Acad. Sci. U.S.A. 91, 2146-2150 (1994).
33. M. H. Hecht, A. Das, A. Go, L. H. Bradley, Y. Wei, De novo proteins from designed combinatorial libraries. Protein Sci. 13, 1711-1723 (2004).
34. M. D. S. Kumar et al., ProTherm and ProNIT: thermodynamic databases for proteins and protein-nucleic acid interactions. Nucleic Acids Res. 34, D204-6 (2006).
35. E. G. Baker et al., Local and macroscopic electrostatic interactions in single α-helices.

Nat. Chem. Biol. 11, 221-228 (2015).
36. D. S. Doering, P. Matsudaira, Cysteine scanning mutagenesis at 40 of 76 positions in villin headpiece maps the F-actin binding site and structural features of the domain.

Biochemistry. 35, 12677-12685 (1996).
37. J. Meng et al., High-resolution crystal structures of villin headpiece and mutants with reduced F-actin binding activity. Biochemistry. 44, 11963-11973 (2005).
38. M. A. Verdecia, M. E. Bowman, K. P. Lu, T. Hunter, J. P. Noel. Structural basis for phosphoserine-proline recognition by group IV WW domains. Nat. Struct. Biol. 7, 639-643 (2000).
39. B. K. Shoichet, W. A. Baase, R. Kuroki, B. W. Matthews, A relationship between protein stability and protein function. Proceedings of the National Academy of Sciences. 92, 452-456 (1995).
40. P. A. Chong, H. Lin. J. L. Wrana, J. D. Forman-Kay, An expanded WW domain recognition motif revealed by the interaction between Smad7 and the E3 ubiquitin ligase Smurf2. J. Biol. Chem. 281, 17069-17075 (2006).
41. E. Aragón et al., Structural basis for the versatile interactions of Smad7 with regulator WW domains in TGF-β Pathways. Structure. 20, 1726-1736 (2012).
42. S. Piana, J. L. Klepeis, D. E. Shaw, Assessing the accuracy of physical models used in protein-folding simulations: quantitative evidence from long molecular dynamics simulations. Curr. Opin. Struct. Biol. 24, 98-105 (2014).
43. J. S. Appelbaum et al., Arginine topology controls escape of minimally cationic proteins from early endosomes to the cytoplasm. Chem. Biol. 19, 819-830 (2012).
44. J. R. LaRochelle, G. B. Cobb, A. Steinauer, E. Rhoades, A. Schepartz, Fluorescence correlation spectroscopy reveals highly efficient cytosolic delivery of certain penta-arg proteins and stapled peptides. J. Am. Chem. Soc. 137, 2536-2541 (2015).
45. P.-S. Huang et al., RosettaRemodel: a generalized framework for flexible backbone protein design. PLoS One. 6, e24109 (2011).
46. A. Leaver-Fay et al., in Methods in Enzymology (2013), pp. 109-143.
47. R. F. Alford et al., The Rosetta all-atom energy function for macromolecular modeling and design (2017), doi:10.1101/106054.
48. F. Pedregosa et al., Scikit-learn: Machine Learning in Python. J. Mach. Learn. Res. 12, 2825-2830 (2011).
49. D. M. Hoover, J. Lubkowski, DNAWorks: an automated method for designing oligonucleotides for PCR-based gene synthesis. Nucleic Acids Res. 30, e43 (2002).
50. L. Benatuil, J. M. Perez, J. Belk, C.-M. Ilsieh, An improved yeast transformation method for the generation of very large human antibody libraries. Protein Eng. Des. Sel. 23, 155-159 (2010).
51. G. Chao et al., Isolating and engineering human antibodies using yeast surface display. Nat. Protoc. 1, 755-768 (2006).
52. J. Zhang, K. Kobert, T. Flouri, A. Stamatakis, PEAR: a fast and accurate Illumina Paired-End reAd mergeR. Bioinformatics. 30, 614-620 (2014).
53. A. Patil, D. Huard, C. J. Fonnesbeck, PyMC: Bayesian Stochastic Modelling in Python. J. Stat. Softw. 35, 1-81 (2010).
54. The Theano Development Team et al., Theano: A Python framework for fast computation of mathematical expressions. arXiv [cs.SC] (2016), (available at http://arxiv.org/abs/1605.02688).
55. D. G. Gibson et al., Enzymatic assembly of DNA molecules up to several hundred kilobases. Nat. Methods. 6, 343-345 (2009),
56. F. W. Studier, Protein production by auto-induction in high density shaking cultures. Protein Expr. Purif 41, 207-234 (2005).
57. C. N. Pace, F. Vajdos, L. Fee, G. Grimsley, T. Gray, How to measure and predict the molar absorption coefficient of a protein. Protein Sci. 4, 2411-2423 (1995).
58. M. M. Santoro, D. W. Bolen, Unfolding free energy changes determined by the linear extrapolation method. 1. Unfolding of phenylmethanesulfonyl alpha-chymotrypsin using different denaturants. Biochemistry. 27, 8063-8068 (1988).
59. V. Y. Orekhov, I. Ibraghimov, M. Billeter, Optimizing resolution in multidimensional NMR by three-way decomposition. J. Biomol. NMR. 27, 165-173 (2003).
60. K. Kazimierczuk, V. Y. Orekhov, Accelerated NMR spectroscopy by using compressed sensing. Angew. Chem. Int. Ed Engl. 50, 5556-5559 (2011).
61. F. Delaglio et al., NMRPipe: a multidimensional spectral processing system based on UNIX pipes. J. Biomol. NMR. 6, 277-293 (1995).
62. A. Lemak et al., A novel strategy for NMR resonance assignment and protein structure determination. J. Biomol. NMR. 49, 27-38 (2011).
63. A. Lemak, C. A. Steren, C. H. Arrowsmith, M. Llinás, Sequence specific resonance assignment via Multicanonical Monte Carlo search using an ABACUS approach. J. Biomol. NMR. 41, 29-41 (2008).
64. P. Güntert, Automated NMR structure calculation with CYANA. Methods Mol. Biol. 278, 353-378 (2004).
65. A. T. Branger et al., Crystallography & NMR system: A new software suite for macromolecular structure determination. Acta Crystallogr. D Bio. Crystalogr. 54, 905-921 (1998).
66. J. P. Linge, M. A. Williams, C. A. E. M. Spronk, Alexandre M J, M. Nilges, Refinement of protein structures in explicit solvent. Proteins: Struct. Funct. Bioinf 50, 496-506 (2003).
67. D. T. Jones, Protein secondary structure prediction based on position-specific scoring matrices. J. Mol. Biol. 292, 195-202 (1999).
68. M. Remmert, A. Biegert, A. Hauser, J. SBding, HHblits: lightning-fast iterative protein sequence searching by HMM-HMM alignment. Nat. Methods. 9, 173-175 (2011).
69. V. Alva, S.-Z. Nam, J. Soding, A. N. Lupas, The MPI bioinformatics Toolkit as an integrative platform for advanced protein sequence and structure analysis. Nucleic Acids Res. 44, W410-W415 (2016).
70. G. E. Crooks, WebLogo: A Sequence Logo Generator. Genome Res. 14, 1188-1190 (2004).
71. Y. Pan et al., Quantitative proteomics reveals the kinetics of trypsin-catalyzed protein digestion. Anal. Bioanal. Chem. 406, 6247-6256 (2014).
72. V. Schellenberger, K. Braune, H. J. Hofmann, H. D. Jakubke, The specificity of chymotrypsin. A statistical analysis of hydrolysis data. Eur. J. Biochem. 199, 623-636 (1991).
73. V. Schellenberger, C. W. Turck, L. Hedstrom. W. J. Rutter, Mapping the S′ subsites of serine proteases using acyl transfer to mixtures of peptide nucleophiles. Biochemistry. 32, 4349-4353 (1993).
74. V. Schellenberger, C. W. Turck, W. J. Rutter, Role of the S′ Subsites in Serine Protease Catalysis. Active-Site Mapping of Rat Chymotrypsin, Rat Trypsin, .alpha.-Lytic Protease, and Cercarial Protease from Schistosoma mansoni. Biochemistry. 33, 4251-4257 (1994).
75. R. A. Laskowski, M. W. MacArthur, D. S. Moss, J. M. Thornton, PROCHECK: a program to check the stereochemical quality of protein structures. J. Appl. Crystallogr. 26, 283-291 (1993).
76. A. Bhattacharya, R. Tejero, G. T. Montelione, Evaluating protein structures determined by structural genomics consortia. Proteins. 66, 778-795 (2007).
77. W. Kabsch, C. Sander, Dictionary of protein secondary structure: Pattern recognition of hydrogen-bonded and geometrical features. Biopolymers. 22, 2577-2637 (1983).
78. Y.-R. Lin et al., Control over overall shape and size in de novo designed proteins. Proc. Natl. Acad. Sci. U.S.A. 112, E5478-85 (2015).
79. O. D. Monera, T. J. Sereda, N. E. Zhou, C. M. Kay, R. S. Hodges, Relationship of sidechain hydrophobicity and α-helical propensity on the stability of the single-stranded amphipathic α-helix J. Pept. Sci. 1, 319-329 (1995).
80. F. Zheng, J. Zhang, G. Grigoryan, Tertiary structural propensities reveal fundamental sequence/structure relationships. Structure. 23, 961-971 (2015).

1. A non-naturally occurring polypeptide comprising
(a) 3-5 secondary structure elements, wherein each secondary structure element is either an α-helix (H domain) of between 10-20 amino acid residues in length or a β-strand (E domain) of between 3-10 amino acid residues in length; and
(b) 2-4 linkers of between 2 to 6 amino acid residues in length connecting adjacent secondary structure elements;
wherein the polypeptide is between 25-50 amino acid residues in length; and
wherein the polypeptide includes no cysteine residues.

2. The polypeptide of claim 1, wherein each H domain is independently between 10-15 amino acids in length.

3. The polypeptide of claim 1, wherein each E domain is independently between 3-7 amino acids in length.

4. The polypeptide of claim 1, wherein the polypeptide is between 30-50, 35-50, 35-45, 40-45, or 40-43 amino acid residues in length.

5. The polypeptide of any claim 1, wherein the polypeptide comprises a secondary structure element arrangement selected from the group consisting of HHH, EHEE, HEEH, and EEHEE.

6. The polypeptide of claim 1, wherein the polypeptide comprises an amino acid sequence having at least 30% identity along its length to the amino acid sequence selected from the group consisting of SEQ ID NOS:1-4000, or a mirror image thereof.

7. The polypeptide of claim 1, wherein the polypeptide comprises an amino acid sequence having at least 50% identity along its length to the amino acid sequence selected from the group consisting of SEQ ID NOS:1-4000, or a mirror image thereof.

8. The polypeptide of claim 1, wherein the polypeptide comprises an amino acid sequence having at least 80% identity along its length to the amino acid sequence selected from the group consisting of SEQ ID NOS:1-4000, or a mirror image thereof.

9. The polypeptide of claim 1, wherein the polypeptide comprises an amino acid sequence having at least 90% identity along its length to the amino acid sequence selected from the group consisting of SEQ ID NOS:1-4000, or a mirror image thereof.

10. The polypeptide of claim 1, wherein the polypeptide comprises an amino acid sequence having at least 95% identity along its length to the amino acid sequence selected from the group consisting of SEQ ID NOS:1-4000, or a mirror image thereof.

11. The polypeptide of claim 1, wherein the polypeptide comprises an amino acid sequence selected from the group consisting of SEQ ID NOS:1-4000, or a mirror image thereof.

12. An isolated nucleic acid encoding the polypeptide of claim 1.

13. A recombinant expression vector comprising the isolated nucleic acid of claim 12 operatively linked to a promoter.

14. A recombinant host cell comprising the recombinant expression vector of claim 13.

15. A method, comprising:
(a) using a computing device to construct a library of proteins, wherein the computing device designs a sequence to stabilize the backbone of the protein, and wherein the proteins comprise less than about 50 amino acids;
(b) synthesizing the proteins using next-generation gene synthesis;
(c) expressing the proteins in yeast so that every cell displays many copies of one protein sequence on its surface; and
(d) screening the library of proteins for susceptibility to digestion by protease.

16. The method of claim 15, wherein the synthesizing step comprises oligo library synthesis technology, capable of parallel synthesis of 104-105 arbitrarily specified DNA sequences long enough to encode the proteins.

17. The method of claim 15, wherein in the screening step, cells are incubated with varying concentrations of protease, those displaying resistant proteins are isolated by fluorescence-activated cell sorting (FACS), and the frequencies of each protein at each protease concentration are determined by deep sequencing.

18. The method claim 15, the method further comprising assigning each protein a stability score, wherein the stability score comprises: the difference between the measured EC₅₀ and the predicted EC₅₀ in the unfolded state of the protein, according to a sequence-based model parameterized using EC₅₀ measurements of scrambled sequences.

19. The method of claim 18, wherein a stability score of 1 corresponds to a 10-fold higher EC₅₀ than the predicted EC₅₀ in the unfolded state.

20. The method of claim 15, wherein the library comprises 1,000 to 30,000 proteins.
